(19) United States
(12) Patent Application Publication
(10) Pub. No.: US 2015/0277225 A1
(43) Pub. Date: Oct. 1, 2015

(54) ACTINIC-RAY-SENSITIVE OR RADIATION-SENSITIVE RESIN COMPOSITION, RESIST FILM FORMED USING SAID COMPOSITION, METHOD FOR FORMING PATTERN USING SAID COMPOSITION, PROCESS FOR PRODUCING ELECTRONIC DEVICE, AND ELECTRONIC DEVICE

(71) Applicant: FUJIFILM CORPORATION, Tokyo (JP)

(72) Inventors: MASAFUMI KOJIMA, SHIZUOKA (JP); AKINORI SHIBUYA, SHIZUOKA (JP); AKIYOSHI GOTO, SHIZUOKA (JP); SHOHEI KATAOKA, SHIZUOKA (JP); KOSUKE KOSHIJIMA, SHIZUOKA (JP)

(21) Appl. No.: 14/738,953

(22) Filed: Jun. 15, 2015

Related U.S. Application Data
(63) Continuation of application No. PCT/JP2013/082616, filed on Dec. 4, 2013.

(30) Foreign Application Priority Data
Dec. 28, 2012 (JP) 2012-288967

(51) Int. Cl. G03F 7/004; C07D 335/02; C07D 333/46; C07D 279/12; C07D 327/06; G03F 7/20

(52) U.S. Cl. CPC G03F 7/0045; C07D 327/06; G03F 7/2041; C07D 333/46; C07D 279/12; C07D 335/02

(57) ABSTRACT

An actinic-ray-sensitive or radiation-sensitive resin composition contains a compound (A) which generates acid by being irradiated with actinic rays or radiation where, when relative light absorbance is εᵣ using triphenyl sulfonium nonaphlate as a reference and relative quantum efficiency is φᵣ using triphenyl sulfonium nonaphlate as a reference, the relative light absorbance εᵣ is 0.4 to 0.8 and εᵣ×φᵣ is 0.5 to 1.0.

### CROSS-REFERENCE TO RELATED APPLICATIONS

This application is a Continuation of PCT International Application No. PCT/JP2013/082616 filed on Dec. 4, 2013, which claims priority under 35 U.S.C §119(a) to Japanese Patent Application No. 2012-288967 filed on Dec. 28, 2012. Each of the above application(s) is hereby expressly incorporated by reference, in its entirety, into the present application.

### BACKGROUND OF THE INVENTION

1. Field of the Invention

The present invention relates to an actinic-ray-sensitive or radiation-sensitive resin composition whose properties change due to a reaction caused by irradiation with actinic rays or radiation, a resist film which is formed using the composition, a pattern forming method which uses the composition, a process for producing an electronic device, and an electronic device. In more detail, the present invention relates to an actinic-ray-sensitive or radiation-sensitive resin composition, which is used in processes for manufacturing semiconductors such as IC, processes for manufacturing circuit substrates such as liquid crystals and thermal heads in addition to other photofabrication processes, lithography printing plates, and acid curable compositions, a resist film which is formed using the composition, a pattern forming method which uses the composition, a process for producing an electronic device, and an electronic device.

2. Description of the Related Art

An actinic-ray-sensitive or radiation-sensitive resin composition is a pattern forming material which generates acid in an exposed section by being irradiated with radiation such as far ultraviolet light, changes solubility with respect to a developing solution in an irradiation section and a non-irradiation section of active radiation according to a reaction which uses the acid as a catalyst, and thus forms a pattern on a substrate.

In a case where a KrF excimer laser is an exposure light source, since a resin which mainly has poly(hydroxy styrene) with small absorption in the 248 nm region as a basic skeleton is used for a main component, a favorable pattern with high sensitivity and high resolution is formed, and the system is favorable compared to a naphthoquinone diazide/novolac resin base in the related art.

On the other hand, in a case of using a light source with even shorter wavelength, for example, an ArF excimer laser (193 nm) as the exposure light source, since a compound which has an aromatic group substantially exhibits great absorption in the 193 nm region, even the chemically amplification system described above is not sufficient. For this reason, a resist for an ArF excimer laser, which contains a resin which has an alicyclic hydrocarbon structure, has been developed.

However, from the point of view of the overall performance as a resist, it is extremely difficult to find an appropriate combination of a resin, a photoacid generator, a basic compound, an additive agent, a solvent, and the like to be used and the overall performance is still not sufficient in practice. For example, there has been a demand for the development of a resist where there are few pattern collapses, where pattern roughness characteristics such as exposure latitude and line width roughness (LWR) are excellent, and where there are few changes in performance due to passing of time.

A photoacid generator which is the main constituent component of an actinic-ray-sensitive or radiation-sensitive resin composition is a compound which generates acid by absorbing light. In the field of photoresist materials, sulfonium salt which is configured by sulfonium cations and counter-anions (X⁻) is widely used as a photoacid generator. Firstly, the sulfonium cations absorb light at the time of exposure. Next, the light energy which is absorbed causes a decomposition reaction in the sulfonium cations. When the sulfonium cations decompose, hydrogen ions (H⁺) are generated, the generated hydrogen ions (H⁺) move to the counter-anions (X⁻), and acid (H⁺X⁻) is generated. In the photoresist materials, due to the effect of the generated acid (H⁺X⁻), the solubility of binder components in a developing solution changes or insolubilization occurs with respect to the developing solution due to a cross-linking reaction taking place. Due to this, there is a contrast in the solubility in the developing solution between an exposed section and a non-exposed section and it is possible to form a nanometer-order pattern.

In general, it is desirable that the photoacid generator generates acid with high efficiency at the time of exposure. Due to this, the sensitivity of the resist film is improved and it is possible to form a pattern with a smaller exposure amount. In order to generate acid with high efficiency, it is preferable that the photoacid generator fulfil two conditions of “high light absorbance (the degree to which the irradiated light is absorbed is large)” and “high decomposition efficiency (the decomposition reaction proceeds with high efficiency due to the light energy which is absorbed). For example, a photoacid generator which has triphenyl sulfonium cations is widely used as a photoacid generator for photoresists since the light absorbance thereof is high.

In addition, other than the photoacid generator which has triphenyl sulfonium cations described above, various photoacid generators are used as a resist composition material for various types of uses and, for example, various photoacid generators are described in JP2012-137697A and US2012/0219913A.

### SUMMARY OF THE INVENTION

However, JP2012-137697A and US2012/0219913A do not describe an actinic-ray-sensitive or radiation-sensitive resin composition where, when forming fine patterns (for example, line widths of 45 nm or less), there are few development defects, where line width roughness and pattern collapse are suppressed, and where it is possible to form a resist pattern with a favorable shape, and there is room for improvement.

In addition, as described above, the photoacid generator is a compound which causes a decomposition reaction and the various physical properties of the photoacid generator greatly influence the preservation stability of the resist. For example, compared to directly after preparation of the resist, the sensitivity of a resist solution decreases after being stored for long periods and there are times when it is not possible to obtain a favorable pattern even in a case of irradiation with the same exposure amount. This decrease is caused by the concentration of the photoacid generator in the resist solution decreasing due to the photoacid generator decomposing due to the passing of time.

In order to secure sufficient sensitivity even after storing the resist solution for long periods, it is effective to increase the added amount of the photoacid generator. Due to this, even in a case where the photoacid generator decomposes during storage, it is possible to generate the necessary amount of generated acid for forming a favorable pattern. However, since the solubility of the photoacid generator in a solvent is not generally large, there are times when the photoacid generator is precipitated as foreign matter (particles) in a case where the added amount is increased. The educted particles are a cause of defects during resist pattern forming.

In addition, demands for reducing development defects in order to improve semiconductor products yields are getting stricter every year and there is a demand for a resist composition which does not generate particles which are a cause of development defects even after being stored for long periods.

For the reasons above, it is difficult to provide a resist solution which has sufficient sensitivity even after being stored for long periods and where the generation of particles is sufficiently suppressed.

In consideration of the related art described above, an object of the present invention is to provide an actinic-ray-sensitive or radiation-sensitive resin composition where, when forming fine patterns (for example, line widths of 45 nm or less), few particles are generated even when storing a resist solution for long periods, additionally, where it is possible to form resist patterns with a favorable shape, where the sensitivity is high even in a case of using a resist solution which is stored for long periods, and where few development defects are generated; a resist film, a pattern forming method, a process for producing an electronic device, an electronic device which use the actinic-ray-sensitive or radiation-sensitive resin composition.

The present inventors completed the present invention as a result of intensive research in order to solve the problems described above.

[1] An actinic-ray-sensitive or radiation-sensitive resin composition containing a compound (A) which generates acid by being irradiated with actinic rays or radiation where, when relative light absorbance is εᵣ using triphenyl sulfonium nonaphlate as a reference and relative quantum efficiency is φᵣ using triphenyl sulfonium nonaphlate as a reference, the relative light absorbance εᵣ is 0.4 to 0.8 and εᵣ×φᵣ is 0.5 to 1.0.

[2] The actinic-ray-sensitive or radiation-sensitive resin composition according to [1] in which the compound (A) is a compound represented by General Formula (1) below.

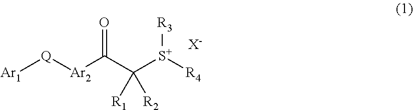

In General Formula (1), Ar₁ and Ar₂ each independently represents an aromatic ring group which has an aromatic ring with 6 to 18 carbon atoms. Ar₁ and Ar₂ may form a ring structure by bonding with each other. Q represents a hetero atom. R₁ and R₂ each independently represents a hydrogen atom, an alkyl group, a cycloalkyl group, a halogen atom, a cyano group, or an aryl group. R₃ and R₄ each independently represents an alkyl group, a cycloalkyl group, a halogen atom, a cyano group, or an aryl group. The R₃ and R₄ may form a ring structure by bonding with each other and the ring structure may include an oxygen atom, a sulfur atom, a ketone group, an ester bond, or an amide bond. X⁻ represents a non-nucleophilic anion.

[3] The actinic-ray-sensitive or radiation-sensitive resin composition according to [2] in which Ar₁ and Ar₂ represent benzene ring groups in General Formula (1).

[4] The actinic-ray-sensitive or radiation-sensitive resin composition according to [2] or [3] in which one of R₁ and R₂ represents a hydrogen atom and the other represents an alkyl group or a cycloalkyl group in General Formula (1).

[5] The actinic-ray-sensitive or radiation-sensitive resin composition according to [1] or [2] in which the compound (A) is a compound which is represented by General Formula (1′) below.

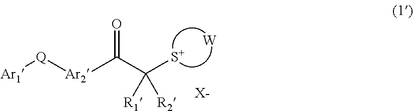

In General Formula (1′), R₁′ is the same as R₁ in General Formula (1). R₂′ is the same as R₂ in General Formula (1).

Ar₁′ is the same as Ar₁ in General Formula (1).

Ar₂′ is the same as Ar₂ in General Formula (1).

W includes an oxygen atom, a sulfur atom, or a nitrogen atom and represents a divalent group which forms a ring structure by linking with sulfonium cations. X⁻ represents a non-nucleophilic anion.

[6] The actinic-ray-sensitive or radiation-sensitive resin composition according to any one of [2] to [5] in which X⁻ in General Formula (1) is a non-nucleophilic anion which is represented by General Formula (2) below.

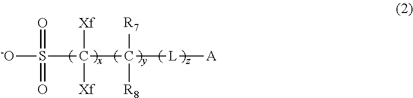

In General Formula (2), a plurality of Xf each independently represents a fluorine atom or an alkyl group which is substituted with at least one fluorine atom.

R₇ and R₈ each independently represents a hydrogen atom, a fluorine atom, or an alkyl group and R₇ and R₈ may be the same or may be different in a case where a plurality of R₇ and R₈ are present. L represents a divalent linking group and L may be the same or may be different in a case where a plurality of L are present. A represents a cyclic organic group. x represents an integer of 1 to 20. y represents an integer of 0 to 10. z represents an integer of 0 to 10.

[7] The actinic-ray-sensitive or radiation-sensitive resin composition according to any one of [1] to [6] further including a resin which is decomposed by an action of an acid and which has increased solubility with respect to an alkaline developing solution.

[8] The actinic-ray-sensitive or radiation-sensitive resin composition according to any one of [1] to [7] further including a low molecular compound which has a nitrogen atom and a group which leaves by an action of an acid or a basic compound.

[9] The actinic-ray-sensitive or radiation-sensitive resin composition according to any one of [1] to [7] further including a basic compound where basicity decreases or disappears by being irradiated with actinic rays or radiation.

[10] A resist film which is formed using the actinic-ray-sensitive or radiation-sensitive resin composition according to any one of [1] to [9].

[11] The resist film according to [10] in which a thickness of the film is 80 nm or less.

[12] A pattern forming method including exposing the resist film according to [10] or [11] and developing the exposed resist film.

[13] The pattern forming method according to [12] in which an exposure method is a liquid immersion exposure method.

[14] A producing method of an electronic device including the pattern forming method according to [12] or [13].

[15] An electronic device manufactured by the producing method of an electronic device according to [14].

According to the present invention, it is possible to provide an actinic-ray-sensitive or radiation-sensitive resin composition where, when forming fine patterns (for example, line widths of 45 nm or less), few particles are generated even when storing a resist solution for long periods, additionally, where it is possible to form a resist pattern with a favorable shape, where the sensitivity is high even in a case of using a resist solution which is stored for long periods, and few development defects are generated, a resist film, a pattern forming method, a producing method of an electronic device, and an electronic device, which use the actinic-ray-sensitive or radiation-sensitive resin composition.

### BRIEF DESCRIPTION OF THE DRAWING

FIG. 1 is a diagram which shows a relationship between an exposure amount and a film thickness which is used in a calculation of a relative quantum efficiency.

### DESCRIPTION OF THE PREFERRED EMBODIMENTS

Detailed description will be given below of embodiments of the present invention.

In the notation of groups (atomic groups) in the present specification, notation which does not specify substituted or unsubstituted includes both groups which do not have a substituent group and groups which have a substituent group. For example, “alkyl group” includes not only an alkyl group which does not have a substituent group (an unsubstituted alkyl group), but also an alkyl group which has a substituent group (a substituted alkyl group).

“Actinic rays” or “radiation” in the present specification has the meaning of, for example, the bright line spectrum of a mercury lamp, far ultraviolet rays which are represented by an excimer laser, extreme ultraviolet rays (EUV light), X-rays, or electron beams (EB). In addition, “light” in the present invention has the meaning of actinic rays or radiation.

In addition, unless otherwise stated, “exposure” in the present specification has the meaning not only of exposure to a mercury lamp, far ultraviolet rays which are represented by excimer laser, X-rays, or EUV light, but also of drawing using particle beams of electron beams, ion beams, or the like.

Examples of indexes which relate to the acid generation efficiency of the compound (A) contained in the actinic-ray-sensitive or radiation-sensitive resin composition of the present invention (also referred to below as a “composition”) include light absorbance ε and quantum efficiency φ.

The light absorbance ε represents the degree to which the acid generating agent absorbs light. High light absorbance has the meaning that the acid generating agent easily absorbs light. In addition, the quantum efficiency φ is a value which represents how much of the light energy which the acid generating agent absorbs is used in the decomposition reaction. High quantum efficiency φ has the meaning that the decomposition reaction proceeds with high efficiency when a certain amount of light energy is absorbed. When the acid generating agent generates acid, two processes are carried out: (1) a process where the acid generating agent absorbs light and (2) a process where a decomposition reaction proceeds. For this reason, it is possible to use ε×φ which is the product of the light absorbance ε and the quantum efficiency φ as an index which indicates the acid generation efficiency of the acid generating agent. The larger the value of ε×φ, the higher the efficiency with which the acid generating agent generates acid.

Here, the relative values of the light absorbance and the quantum efficiency of the acid generating agent in a case where the light absorbance ε and the quantum efficiency φ of triphenyl sulfonium nonaphlate are set as 1 are respectively a relative light absorbance εᵣ and a relative quantum efficiency φᵣ.

The actinic-ray-sensitive or radiation-sensitive resin composition of the present invention contains a compound (A) (also referred to below as a “compound (A)”) which generates acid by being irradiated with actinic rays or radiation in which, when the relative light absorbance is εᵣ and the relative quantum efficiency is φᵣ using triphenyl sulfonium nonaphlate as a reference, the relative light absorbance εᵣ is 0.4 to 0.8 and εᵣ×φᵣ is 0.5 to 1.0.

According to the actinic-ray-sensitive or radiation-sensitive resin composition according to the present invention, it is possible to provide an actinic-ray-sensitive or radiation-sensitive resin composition according to the present invention where, when forming fine patterns (for example, line widths of 45 nm or less), few particles are generated even when storing a resist solution for long periods, additionally, where it is possible to form a resist pattern with a favorable shape, where the sensitivity is high even in a case of using a resist solution which is stored for long periods, and where few development defects are generated. The reasons therefor are not certain, but are thought to be as follows.

As described above, an acid generating agent where the relative light absorbance exceeds 0.8 using triphenyl sulfonium nonaphlate as a reference is widely known as an acid generating agent in resist compositions; however, it is considered that, since the light absorbance in ArF exposure is high, it is not possible to sufficiently expose a bottom section of a resist film and the pattern shape deteriorates as a result.

On the other hand, an acid generating agent where the relative light absorbance is less than 0.4 using triphenyl sulfonium nonaphlate as a reference is also known; however, such an acid generating agent needs to be contained in an actinic-ray-sensitive or radiation-sensitive resin composition at a high concentration to suppress decreases in the sensitivity of the resist solution along with decreases in the content after being stored for long periods due to the decomposition reaction of the acid generating agent. Here, it is considered that, since the solubility of the acid generating agent in a solvent is not high, there are times when the acid generating agent is precipitated as particles after being stored for long periods and the particles are a cause of development defects.

With respect thereto, firstly, the relative light absorbance of the compound (A) contained in the actinic-ray-sensitive or radiation-sensitive resin composition according to the present invention is 0.8 or less using triphenyl sulfonium nonaphlate as a reference.

Due to this, it is considered that the bottom section of the resist film is sufficiently exposed during a pattern forming and as a result, a pattern shape improves.

Furthermore, the relative light absorbance εᵣ of the compound (A) contained in the actinic-ray-sensitive or radiation-sensitive resin composition according to the present invention is 0.4 or more using triphenyl sulfonium nonaphlate as a reference.

Due to this, it is considered that, compared to an acid generating agent where the relative light absorbance is less than 0.4, it is not necessary for the actinic-ray-sensitive or radiation-sensitive resin composition to contain an excess of the compound (A), precipitation of the acid generating agent being as particles after storing the resist solution for long periods is suppressed, and development defects are reduced as a result even when performing pattern forming using a resist solution stored for long periods.

In addition, acid generating agents are known in which, when the relative light absorbance is εᵣ and the relative quantum efficiency is φᵣ using triphenyl sulfonium nonaphlate as a reference, εᵣ×φᵣ is less than 0.5; however, it is considered that the acid generation efficiency thereof is low and the sensitivity of the resist solution decreases and, additionally, as described above, since it is necessary for the actinic-ray-sensitive or radiation-sensitive resin composition to contain a large amount of the acid generating agent, development defects due to generation of particles after storing the resist solution for long periods increase as a result. That is, it is considered that, in a case where the actinic-ray-sensitive or radiation-sensitive resin composition contains the same amount of the acid generating agent where the εᵣ×φᵣ is less than 0.5 described above as the compound (A) of the present invention, the generation of particles after storing the resist solution for long periods is suppressed and development defects are also reduced, but the sensitivity of the resist solution decreases.

With respect thereto, εᵣ×φᵣ of the compound (A) contained in the actinic-ray-sensitive or radiation-sensitive resin composition according to the present invention is 0.5 or more using triphenyl sulfonium nonaphlate as a reference.

Due to this, it is considered that, since high sensitivity is maintained even after storing the resist solution for long periods and it is not necessary for the actinic-ray-sensitive or radiation-sensitive resin composition to contain an excess of the compound (A), for the reasons described above, the precipitation of the acid generating agent as particles after storing a resist solution for long periods is suppressed, and development defects are reduced even in a case of performing pattern forming using a resist solution stored for long periods as a result.

In addition, since it is difficult to obtain or synthesize a photoacid generator where, when relative light absorbance is εᵣ and relative quantum efficiency is φᵣ using triphenyl sulfonium nonaphlate as a reference, εᵣ×φᵣ exceeds 1.0, εᵣ×φᵣ is 1.0 or less in the present invention.

As described above, by containing the compound (A) which is not known in the art and which generates acid by being irradiated with actinic rays or radiation where, when relative light absorbance is εᵣ and relative quantum efficiency is φᵣ using triphenyl sulfonium nonaphlate as a reference, the relative light absorbance εᵣ is 0.4 to 0.8 and εᵣ×φᵣ is 0.5 to 1.0, an actinic-ray-sensitive or radiation-sensitive resin composition is provided where few particles are generated even when storing a resist solution for long periods, additionally, where it is possible to form a resist pattern with a favorable shape, where the sensitivity is high even in a case of using a resist solution which is stored for long periods, and where few development defects are generated.

Description will be given below of the actinic-ray-sensitive or radiation-sensitive resin composition according to the present invention.

The actinic-ray-sensitive or radiation-sensitive resin composition of the present invention is typically a resist composition and may be a negative type resist composition (that is, a resist composition for organic solvent development) or may be a positive type resist composition. In addition, the composition according to the present invention is typically a chemical amplification-type resist composition.

[1] Compound (A) which generates acid by being irradiated with actinic rays or radiation where, when relative light absorbance is εᵣ and relative quantum efficiency is φᵣ using triphenyl sulfonium nonaphlate as a reference, the relative light absorbance εᵣ is 0.4 to 0.8 and εᵣ×φᵣ is 0.5 to 1.0.

The actinic-ray-sensitive or radiation-sensitive resin composition of the present invention contains a compound (A) (also referred to below as a “compound (A)”) which generates acid by being irradiated with actinic rays or radiation where, when relative light absorbance is εᵣ and relative quantum efficiency is φᵣ using triphenyl sulfonium nonaphlate as a reference, the relative light absorbance εᵣ is 0.4 to 0.8 and εᵣ×φᵣ is 0.5 to 1.0 as described above.

The relative light absorbance εᵣ of the acid generating agent is 0.4 to 0.8, 0.45 to 0.7 is preferable, 0.5 to 0.65 is more preferable, and 0.55 to 0.6 is even more preferable.

εᵣ×φᵣ of the acid generating agent is 0.5 to 1.0, 0.55 to 0.9 is preferable, 0.6 to 0.8 is more preferable, and 0.65 to 0.7 is even more preferable.

The relative light absorbance εᵣ of the acid generating agent is a value which is standardized by setting the molar absorbance coefficient ε_{TPS} of triphenyl sulfonium nonaphlate as 1 and, specifically, is a value which is calculated using the formula below.

εᵣ=ε_{z}/ε_{TPS}

In the formula, εᵣ represents the relative light absorbance of the acid generating agent.

ε_{z} represents the molar absorbance coefficient of the acid generating agent.

ε_{TPS} represents the molar absorbance coefficient of triphenyl sulfonium nonaphlate.

A cell is used to measure the UV spectrum with regard to a measurement solution in which the acid generating agent is dissolved in a solvent, and the molar absorbance coefficient of the target acid generating agent is calculated according to the Lambert-Beer formula from light absorbance (A) with respect to light with wavelength of 193 nm and the measured solvent concentration (C).

The relative quantum efficiency φᵣ of the acid generating agent is a value which is standardized by setting the absorbance coefficient ε_{TPS} and the quantum efficiency φ_{TPS} of triphenyl sulfonium nonaphlate as 1 and, specifically, is calculated using the formula below.

φᵣ=(φ_{TPS}×ε_{TPS}×E_{TPS})/(εᵣ×Eᵣ)

In the formula described above, ε_{TPS} and φ_{TPS} are 1.

E_{TPS} represents the sensitivity of triphenyl sulfonium nonaphlate.

Eᵣ represents the sensitivity of the acid generating agent.

εᵣ represents the relative light absorbance of the acid generating agent which is calculated by the method described above.

φᵣ represents the relative quantum efficiency of the acid generating agent.

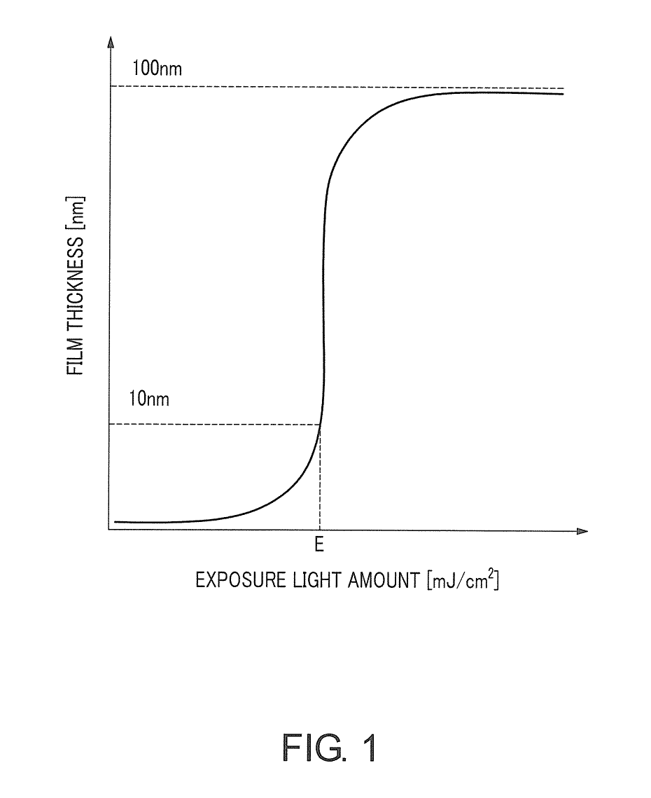

Here, the formula which is represented by φᵣ=(φ_{TPS}×ε_{TPS}×E_{TPS})/(εᵣ×Eᵣ) is derived from the idea that “in the rising portion of curved line of the ‘exposure amount with respect to film thickness’ shown in FIG. 1, the amount of generated acid is a constant value”.

The sensitivity E_{TPS} of triphenyl sulfonium nonaphlate which is used for the calculation of φᵣ of the acid generating agent is calculated by the method below.

Firstly, a resist solution with a solid content concentration of 3.5 mass % is obtained by dissolving 10 g of Polymer (1) described below, 0.3 g of a basic compound DIA (2,6-diisopropyl aniline), and 2.0 g of triphenyl sulfonium nonaphlate in a solvent (PGMEA).

A resist film with a film thickness of 100 nm is formed using the obtained resist solution and exposure is performed using an ArF excimer laser scanner.

After that, heating is carried out at 100° C. for 60 seconds, developing is subsequently carried out by paddling in butyl acetate for 30 seconds, rinsing is performed with methyl isobutyl carbinol (MIBC), and baking is performed at 90° C. for 60 seconds.

By increasing the exposure amount in steps of from 1 mJ/cm² to 0.3 mJ/cm², the exposure amount when the film thickness after baking is 10 nm or more is defined as the sensitivity E_{TPS} of triphenyl sulfonium nonaphlate.

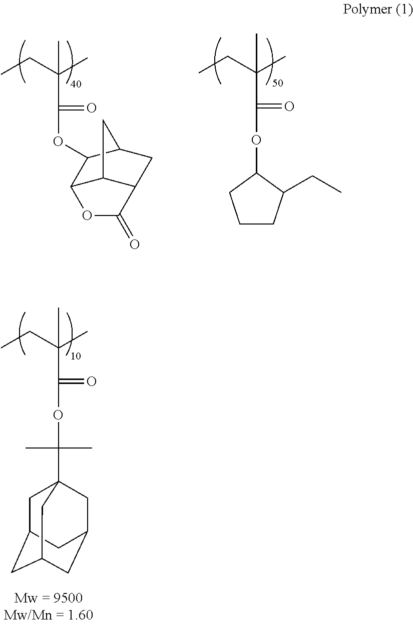

The sensitivity Eᵣ of acid generating agents other than triphenyl sulfonium nonaphlate is measured in the same manner as the measurement of the sensitivity E_{TPS} except that triphenyl sulfonium nonaphlate is changed to the compound (A) in the measurement of the sensitivity E_{TPS} described above.

The compound (A) is not particularly limited; however, from the point of view of fulfilling the parameters described above, a compound which is represented by General Formula (1) described below is more preferable.

It is possible to fulfil the parameters described above by adjusting the cation structure of the compound (A) to be a specific structure; however, the parameters described above are more reliably fulfilled by the compound (A) being a compound which is represented by General Formula (1) described below.

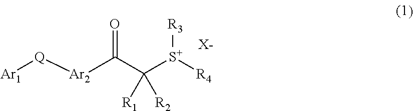

In General Formula (1), Ar₁ and Ar₂ each independently represents an aromatic ring group which has an aromatic ring. Q represents a hetero atom. R₁ and R₂ each independently represents a hydrogen atom, an alkyl group, a cycloalkyl group, a halogen atom, a cyano group, or an aryl group. R₃ and R₄ each independently represents an alkyl group, a cycloalkyl group, a halogen atom, a cyano group, or an aryl group. The R₃ and R₄ may form a ring structure by bonding with each other and the ring structure may include an oxygen atom, a sulfur atom, a ketone group, an ester bond, or an amide bond. X⁻ represents a non-nucleophilic anion.

The aromatic ring group which has an aromatic ring which Ar₁ and Ar₂ each independently represents is an aromatic ring group which has an aromatic ring with 6 to 18 carbon atoms, examples thereof include a benzene ring group, a naphthalene ring group, a biphenyl ring group, and the like, and a benzene ring group is preferable.

Here, the aromatic ring with 6 to 18 carbon atoms has the meaning that the number of carbon atoms which configure ring members of the aromatic ring is 6 to 18 and the number of carbon atoms in substituent groups where the carbon atoms described above may have an aromatic ring is not included.

Thus, by the number of carbon atoms of the aromatic ring of the aromatic ring group in Ar₁ and Ar₂ being 6 to 18, the relative light absorbance εᵣ is within a range of 0.4 to 0.8 and εᵣ×φᵣ is within a range of 0.5 to 1.0. The aromatic ring group which has an aromatic ring may have a substituent group and examples of preferable substituent groups include an alkyl group, a cycloalkyl group, an alkoxy group, a cycloalkoxy group, an aryloxy group, an alkylthio group, an arylthio group, a hydroxyl group, and a halogen atom (preferably a fluorine atom) and examples of more preferable substituent groups include an alkoxy group. The substituent groups on the aromatic ring groups of Ar₁ and Ar₂ may form a ring by linking with each other.

Furthermore, Ar₁ and Ar₂ may be linked with each other without a substituent group being interposed.

An alkyl group as a substituent group in a case where the aromatic ring group of Ar₁ and Ar₂ has a substituent group is preferably a linear or branched alkyl group with 1 to 20 carbon atoms and may have an oxygen atom, a sulfur atom, and a nitrogen atom in the alkyl chain. In detail, examples thereof include linear alkyl groups such as a methyl group, an ethyl group, an n-propyl group, an n-butyl group, an n-pentyl group, an n-hexyl group, an n-octyl group, an n-dodecyl group, an n-tetradecyl group, and an n-octadecyl group and branched alkyl groups such as an isopropyl group, an isobutyl group, a t-butyl group, a neopentyl group, and a 2-ethylhexyl group. The alkyl group may have a substituent group and examples of an alkyl group which has a substituent group include a cyanomethyl group, 2,2,2-trifluoroethyl group, a methoxycarbonyl methyl group, an ethoxycarbonyl methyl group, and the like.

A cycloalkyl group as the substituent group in a case where the aromatic ring group of Ar₁ and Ar₂ has a substituent group is preferably a cycloalkyl group with 3 to 20 carbon atoms and may have an oxygen atom or a sulfur atom in the ring. In detail, examples thereof include a cyclopropyl group, a cyclopentyl group, a cyclohexyl group, a norbornyl group, an adamantyl group, and the like. The cycloalkyl group may have a substituent group and examples of the substituent group include an alkyl group and an alkoxy group.

The alkoxy group as a substituent group in a case where the aromatic ring group of Ar₁ and Ar₂ has a substituent group is preferably an alkoxy group with 1 to 20 carbon atoms. In detail, examples thereof include a methoxy group, an ethoxy group, an isopropyloxy group, a t-butyloxy group, a t-amyloxy group, and an n-butyloxy group. The alkoxy group may have a substituent group and examples of the substituent group include an alkyl group and a cycloalkyl group.

A cycloalkoxy group as a substituent group in a case where the aromatic ring group of Ar₁ and Ar₂ has a substituent group is preferably a cycloalkoxy group with 3 to 20 carbon atoms and examples thereof include a cyclohexyloxy group, a norbornyloxy group, an adamantyloxy group, and the like. The cycloalkoxy group may have a substituent group and examples of the substituent group include an alkyl group and a cycloalkyl group.

An aryloxy group and an aryl group on an arylthio group as the substituent group in a case where the aromatic ring group of Ar₁ and Ar₂ has a substituent group is preferably an aryl group with 6 to 14 carbon atoms and examples thereof include a phenyl group, a naphthyl group, a biphenyl group, and the like. The aryl group may have a substituent group and examples of the preferable substituent group include an alkyl group, a cycloalkyl group, an alkoxy group, a cycloalkoxy group, an aryloxy group, an alkylthio group, an arylthio group, a hydroxyl group, and a halogen atom.

The definition and preferable range of the alkyl group on the alkylthio group as the substituent group in a case where the aromatic ring group of Ar₁ and Ar₂ has a substituent group are the same as for the alkyl group as a substituent group in a case where the aromatic ring group of Ar₁ and Ar₂ has a substituent group.

R₁ and R₂ each independently represents a hydrogen atom, an alkyl group, a cycloalkyl group, a halogen atom (preferably a fluorine atom), a cyano group, or an aryl group.

The definition and preferable range of the alkyl group represented by R₁ and R₂ are the same as for the alkyl group as a substituent group in a case where the aromatic ring group of Ar₁ and Ar₂ has a substituent group.

The definition and preferable range of the cycloalkyl group represented by R₁ and R₂ are the same as for the cycloalkyl group as the substituent group in a case where the aromatic ring group of Ar₁ and Ar₂ has a substituent group.

The definition and preferable range of the aryl group represented by R₁ and R₂ are the same as for the aryloxy group and the aryl group on the arylthio group as the substituent group in a case where the aromatic ring group of Ar₁ and Ar₂ has a substituent group.

R₁ and R₂ are preferably a hydrogen atom, an alkyl group, or a cycloalkyl group, more preferably a hydrogen atom, a t-butyl group, a cyclopentyl group, or a cyclohexyl group. One of R₁ and R₂, even more preferably, represents a hydrogen atom and the other is a t-butyl group, a cyclopentyl group, or a cyclohexyl group.

R₃ and R₄ each independently represents an alkyl group, a cycloalkyl group, a halogen atom (preferably a fluorine atom), a cyano group, or an aryl group. The R₃ and R₄ may form a ring structure by bonding with each other and the ring structure may include a nitrogen atom, an oxygen atom, a sulfur atom, a ketone group, an ester bond, or an amide bond. X⁻ represents a non-nucleophilic anion.

The definition and preferable range of the alkyl group represented by R₃ and R₄ are the same as for the alkyl group as the substituent group in a case where the aromatic ring group of Ar₁ and Ar₂ has a substituent group.

The definition and preferable range of the cycloalkyl group represented by R₃ and R₄ are the same as for the cycloalkyl group as the substituent group in a case where the aromatic ring group of Ar₁ and Ar₂ has a substituent group.

The definition and preferable range of the aryl group represented by R₃ and R₄ are the same as for the aryloxy group and the aryl group on the arylthio group as the substituent group in a case where the aromatic ring group of Ar₁ and Ar₂ has a substituent group.

In a case where R₃ and R₄ form a ring structure by bonding with each other, the ring structure may include an oxygen atom, a nitrogen atom, a sulfur atom, a ketone group, an ester bond, or an amide bond and an oxygen atom or a ketone group are preferably included.

R₃ and R₄ are preferably alkyl groups.

In a case where R₃ and R₄ form a ring structure by bonding with each other, preferable examples of R₃ and R₄ include an alkylene group or an alkylene group which includes an oxygen atom and the number of carbon atoms of the alkylene group described above is preferably 1 to 4, more preferably 2 or 3, and particularly preferably 2.

More preferably, the compound (A) is a compound which is represented by General Formula (1′) below.

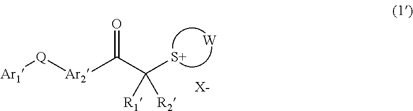

In General Formula (1′), the definition and preferable range of R₁′ are the same as in R₁ in General Formula (1) described above.

The definition and preferable range of R₂′ are the same as in R₂ in General Formula (1) described above.

The definition and preferable range of Ar₁′ are the same as in Ar₁ in General Formula (1) described above.

The definition and preferable range of Ar₂′ are the same as in Ar₂ in General Formula (1) described above.

W includes an oxygen atom, a sulfur atom, or a nitrogen atom and represents a divalent group which forms a ring structure by linking with sulfonium cations (S⁺ in General Formula (1)). In a case of including a nitrogen atom, it is preferable that W is a group where the basicity of the nitrogen atom is low or which does not have basicity and a group which has a nitrogen atom which is substituted with an electron-withdrawing group such as an amide structure, a carbamate structure, and a sulfonamide structure is preferable. The electron-withdrawing group which is substituted with the nitrogen atom may be an ester group.

X⁻ represents a non-nucleophilic anion. The preferable range of X⁻ is the same as for X⁻ in General Formula (1).

Preferable examples of W include a divalent group which includes an oxygen atom or a nitrogen atom and which forms a ring structure by linking with S⁺ and particularly preferable examples thereof include an alkylene group which includes an oxygen atom or an alkylene group which includes a structure which is represented by General Formula (IV) below. In Formula (IV), the nitrogen atom N is preferably a constituent atom of a ring which is formed by linking with S⁺ in General Formula (1).

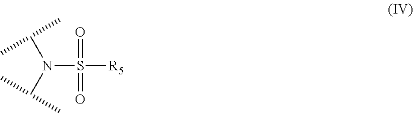

In Formula (IV), R₅ represents an alkyl group, a cycloalkyl group, or an aryl group, and is preferably an alkyl group. Specific examples and preferable examples of the alkyl group, the cycloalkyl group, and the aryl group with regard to R₅ include the same groups as the specific examples and preferable examples of the alkyl group, the cycloalkyl group, and the aryl group in R₁ described above.

The oxygen atom, the sulfur atom, or the nitrogen atom which is included in W may be linked with S⁺ in General Formula (1) via a divalent linking group. Examples of the divalent linking group include an alkylene group and an alkylene group which includes an oxygen atom, and the like. The number of carbons atoms of the alkylene group is preferably 1 to 4, more preferably 2 or 3, and particularly preferably 2.

More preferably, the compound (A) is a compound which is represented by General Formula (1a) or (1b) below.

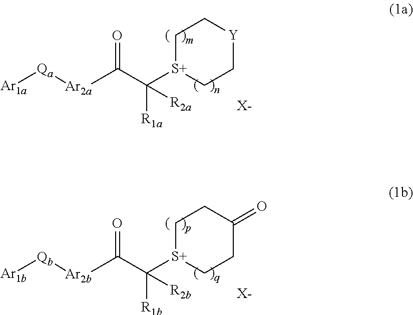

Ar₁ₐ, Ar₂ₐ, Qₐ, R₁ₐ, R₂ₐ, and X⁻ in General Formula (1a) are the same as Ar₁, Ar₂, Q, R₁, R₂, and X⁻ in General Formula (1) described above.

Y represents an oxygen atom and a sulfur atom and an oxygen atom is preferable. m and n are integers and 0 to 3 is preferable, 1 or 2 is more preferable, and 1 is particularly preferable. An alkylene group which links S⁺ and Y may have a substituent group and examples of a preferable substituent group include an alkyl group.

Ar_{1b}, Ar_{2b}, Q_{b}, R_{1b}, R_{2b}, and X⁻ in General Formula (1b) are the same as Ar₁, Ar₂, Q, R₁, R₂, and X⁻ in General Formula (1) described above.

p and q are the same as m and n in General Formula (1a) described above.

More preferably, the compound (A) is a compound which is represented by General Formulae (1a′) and (1b′).

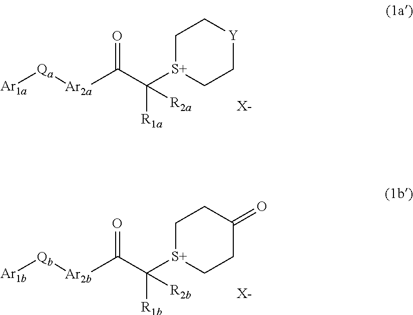

Ar₁ₐ, Ar₂ₐ, Qₐ, R₁ₐ, R₂ₐ, Y, Ar_{1b}, Ar_{2b}, Q_{b}, R_{1b}, R_{2b}, and X⁻ in General Formulae (la′) and (1 b′) are the same as defined in General Formulae (1a) and (1b) described above.

In one aspect of the present invention, a non-nucleophilic anion of X⁻ is preferably a non-nucleophilic anion which is represented by General Formula (2). In this case, it is estimated that improvement in the exposure latitude is further promoted since the volume of generated acid is large and diffusion of the acid is suppressed.

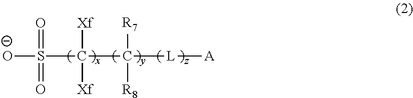

In General Formula (2), a plurality of Xf each independently represents a fluorine atom or an alkyl group which is substituted with at least one fluorine atom.

R₇ and R₈ each independently represents a hydrogen atom, a fluorine atom, or an alkyl group and R₇ and R₈ may be the same or may be different in a case where a plurality thereof are present. L represents a divalent linking group and L may be the same or may be different in a case where a plurality thereof are present. A represents a cyclic organic group. x represents an integer of 1 to 20. y represents an integer of 0 to 10. z represents an integer of 0 to 10.

More detailed description will be given of the non-nucleophilic anion in General Formula (2).

Xf is a fluorine atom or an alkyl group which is substituted with at least one fluorine atom as described above and as the alkyl group in the alkyl group which is substituted with a fluorine atom, an alkyl group with 1 to 10 carbon atoms is preferable and an alkyl group with 1 to 4 carbon atoms is more preferable. In addition, an alkyl group which is substituted with the fluorine atom of Xf is preferably a perfluoroalkyl group.

Xf is preferably a fluorine atom or a perfluoroalkyl group with 1 to 4 carbon atoms. Specific examples thereof include a fluorine atom, CF₃, C₂F₅, C₃F₇, C₄F₉, C₅F₁₁, C₆F₁₃, C₇F₁₅, C₈F₁₇, CH₂CF₃, CH₂CH₂CF₃, CH₂C₂F₅, CH₂CH₂C₂F₅, CH₂C₃F₇, CH₂CH₂C₃F₇, CH₂C₄F₉, and CH₂CH₂C₄F₉ and among these, the fluorine atom and CF₃ are preferable. In particular, it is preferable that all of the Xf are fluorine atoms.

R₇ and R₈ represent a hydrogen atom, a fluorine atom, and an alkyl group as described above and the alkyl group is preferably an alkyl group with 1 to 4 carbon atoms. The alkyl group may be substituted with a fluorine atom. R₇ and R₈ are preferably a hydrogen atom or an unsubstituted alkyl group.

L represents a divalent linking group and examples thereof include —COO—, —OCO—, —CO—, —O—, —S—, —SO—, —SO₂—, —N(Ri)- (in the formula, Ri represents a hydrogen atom or alkyl group), an alkylene group (preferably an alkyl group with 1 to 6 carbon atoms, more preferably an alkyl group with 1 to 4 carbon atoms, particularly preferably a methyl group or an ethyl group, most preferably a methyl group), a cycloalkylene group (preferably with 3 to 10 carbon atoms), an alkenylene group (preferably 2 to 6 carbon atoms), a divalent linking group where a plurality of these are combined, or the like, and —COO—, —OCO—, —CO—, —SO₂—, —CON(Ri)-, —SO₂N(Ri)-, —CON(Ri)-alkylene group-, —N(Ri)CO-alkylene group-, —COO-alkylene group-, or —OCO-alkylene group- are preferable, —SO₂—, —COO—, —OCO—, —COO-alkylene group-, or —OCO-alkylene group- are more preferable. As the alkylene group in the —CON(Ri)-alkylene group-, —N(Ri)CO-alkylene group-, —COO-alkylene group-, and —OCO-alkylene group-, an alkylene group with 1 to 20 carbon atoms is preferable and an alkylene group with 1 to 10 carbon atoms is more preferable. L may be the same or may be different in a case where a plurality thereof are present.

Specific examples and preferable examples of the alkyl group with regard to R₇ and R₈ include the same specific examples and preferable examples described above as R₁ to R₄ in General Formula (1).

The cyclic organic group of A (an organic group which includes a cyclic structure) is not particularly limited as long as the cyclic organic group has a ring structure and examples thereof include an alicyclic group, an aryl group, a heterocyclic group (including not only a heterocyclic group which has aromaticity, but also a heterocyclic group which does not have aromaticity, for example, also including a tetrahydropyran ring structure, a lactone ring structure, and a sultone ring structure), and the like.

The alicyclic group may be either monocyclic or polycyclic and a monocyclic cycloalkyl group such as a cyclopentyl group, a cyclohexyl group, and a cyclooctyl group and a polycyclic cycloalkyl group such as a norbornyl group, a norbornene-il group, a tricyclodecanyl group (for example, a tricyclo[5.2.1.0(2,6)]decanyl group), a tetracyclodecanyl group, a tetracyclododecanyl group, and an adamantyl group are preferable and an adamantyl group is particularly preferable. In addition, a nitrogen atom-containing alicyclic group such as a piperidine group, a decahydroquinoline group, and a decahydroisoquinoline group is also preferable. Among these, an alicyclic group such as a norbornyl group, a tricyclodecanyl group, a tetracyclodecanyl group, a tetracyclododecanyl group, an adamantyl group, a decahydroquinoline group, or a decahydroisoquinoline group which has a bulky structure with 7 or more carbon atoms is preferable from the point of view that it is possible to suppress the in-film diffusibility in a PEB process (heating after exposure) and to improve exposure latitude. Among these, an adamantyl group and a decahydroisoquinoline group are particularly preferable.

Examples of an aryl group include a benzene ring, a naphthalene ring, a phenanthrene ring, and an anthracene ring. Among these, naphthalene with low light absorbance is preferable from the point of view of the light absorbance at 193 nm.

Examples of a heterocyclic group include a heterocyclic group derived from a furan ring, a thiophene ring, a benzofuran ring, a benzothiophene ring, a dibenzofuran ring, a dibenzothiophene ring, a pyridine ring, and a piperidine ring. Among these, a heterocyclic group derived from a furan ring, a thiophene ring, a pyridine ring, and a piperidine ring is preferable. Other examples of a preferable heterocyclic group include the structure shown below (in the formula, X represents a methylene group or an oxygen atom and R represents a monovalent organic group).

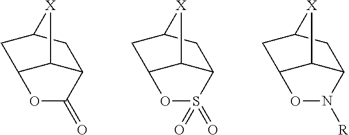

The cyclic organic group of A described above may have a substituent group and examples of the substituent group include an alkyl group (the alkyl group may be any of linear, branched, or cyclic and the number of carbon atoms is preferably 1 to 12), an aryl group (the number of carbon atoms is preferably 6 to 14), a hydroxy group, an alkoxy group, an ester group, an amide group, a urethane group, a ureide group, a thioether group, a sulfonamide group, a sulfonic acid ester group, and the like.

Here, the carbon which configures an organic group which includes a ring structure (carbons which contribute to the ring forming) may be carbonyl carbon.

x in General Formula (2) is preferably 1 to 8, more preferably 1 to 4, and particularly preferably 1. y is preferably 0 to 4, more preferably 0 or 1, and even more preferably 1. z is preferably 0 to 8, more preferably 0 to 4, and even more preferably 1.

In addition, in another aspect of the present invention, the non-nucleophilic anion of X⁻ may be a disulfonyl imidic acid anion.

The disulfonyl imidic acid anion is preferably a bis(alkylsulfonyl) imide anion.

The alkyl group in the bis(alkylsulfonyl) imide anion preferably has 1 to 5 carbon atoms.

Two alkyl groups in the bis(alkylsulfonyl) imide anion may form an alkylene group (preferably with 2 to 4 carbon atoms) by linking with each other and form a ring with an imide group and two sulfonyl groups. The ring structure described above which the bis(alkylsulfonyl) imide anion may form is preferably a ring with 5 to 7 members, more preferably a ring with 6 members.

The alkyl group and the alkylene group formed by two alkyl groups linking with each other may have a substituent group and examples of substituent groups which the alkyl group may have include a halogen atom, an alkyl group which is substituted with a halogen atom, an alkoxy group, an alkylthio group, an alkyloxysulfonyl group, an aryloxysulfonyl group, a cycloalkyl aryloxysulfonyl group, and the like, and a fluorine atom or an alkyl group which is substituted with a fluorine atom is preferable.

From the point of view of the acid strength, the pKa of the generated acid is preferably −1 or less in order to improve the sensitivity.

Here, the compound (A) may be a compound which has a plurality of structures which are represented by General Formula (1).

With regard to the compound which is represented by General Formula (1), the fluorine content ratio which is represented by (total mass of all fluorine atoms included in a compound)/(total mass of all atoms included in the compound) is preferably 0.25 or less, more preferably 0.20 or less, even more preferably 0.15 or less, and particularly preferably 0.10 or less.

Preferable specific examples of the compound (A) will be given below; however, the present invention is not limited thereto.

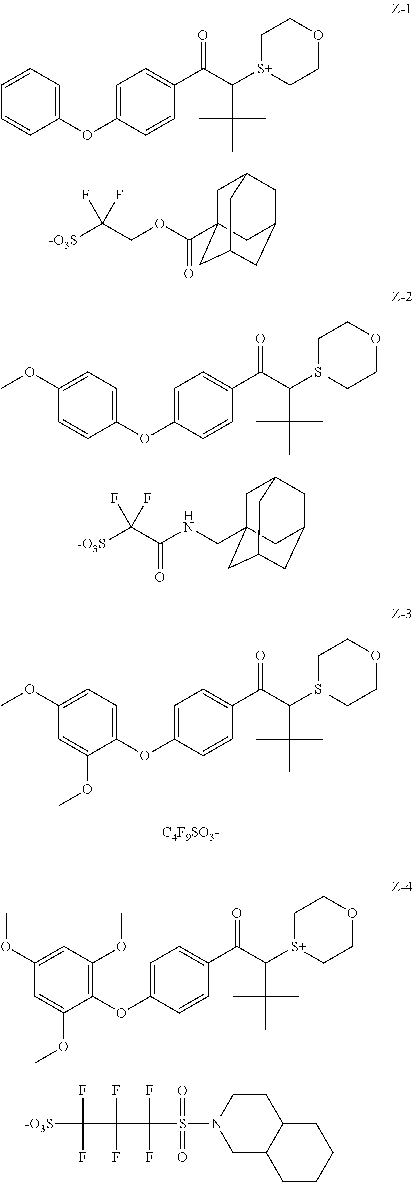

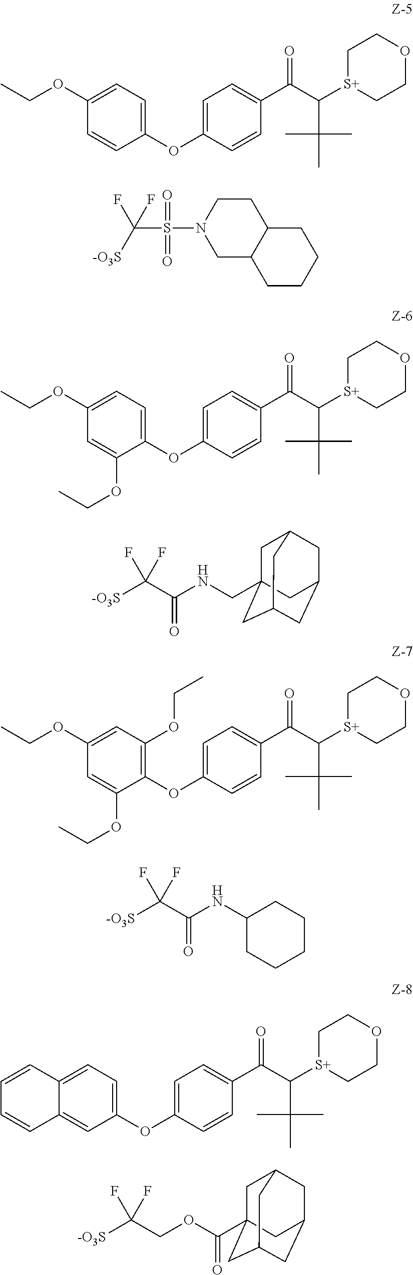

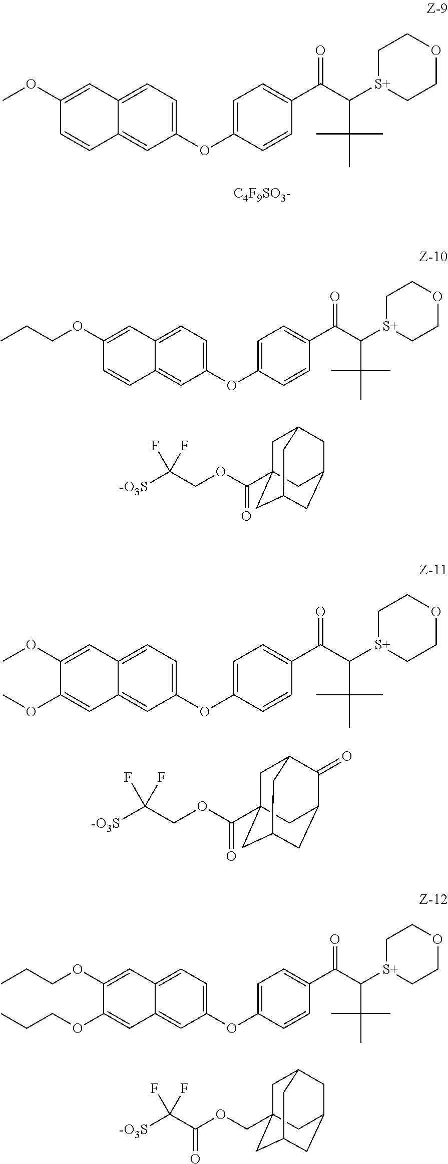

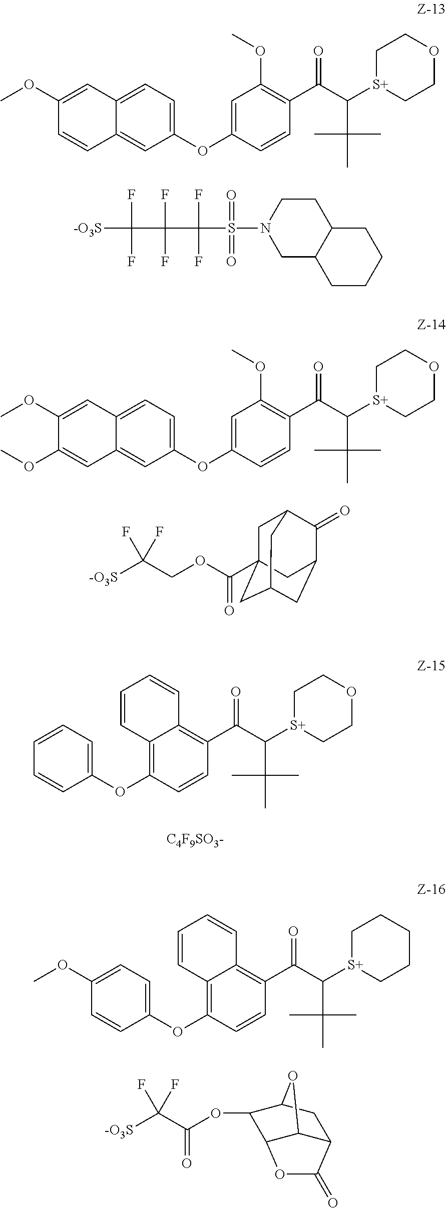

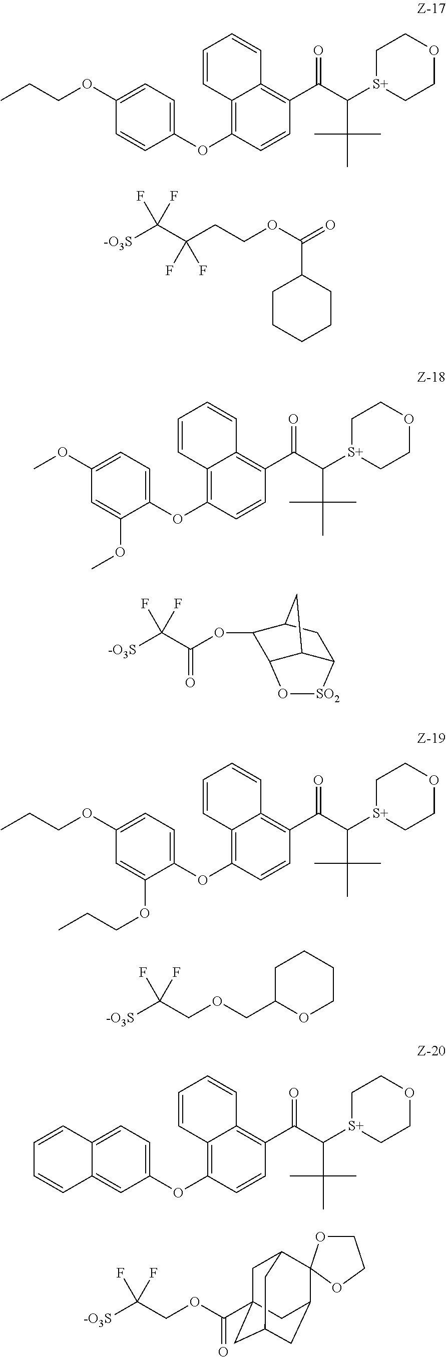

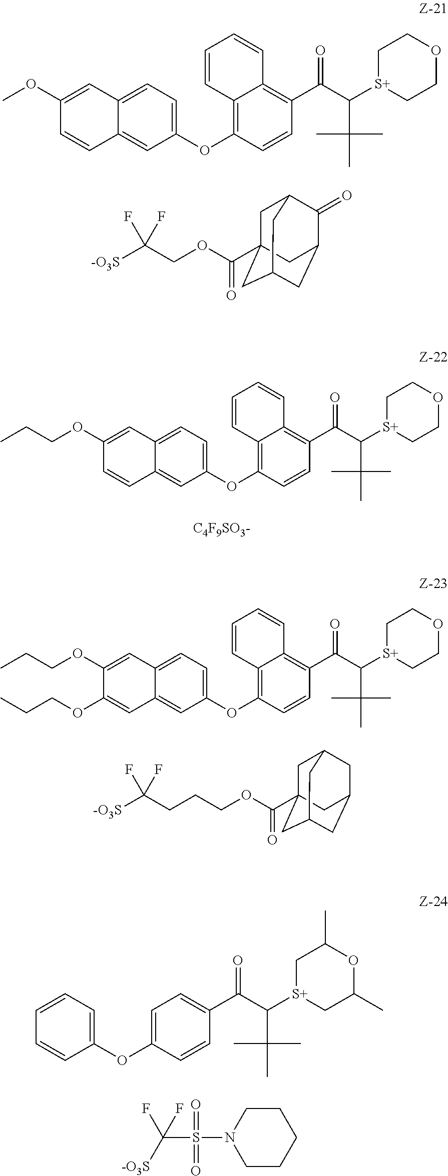

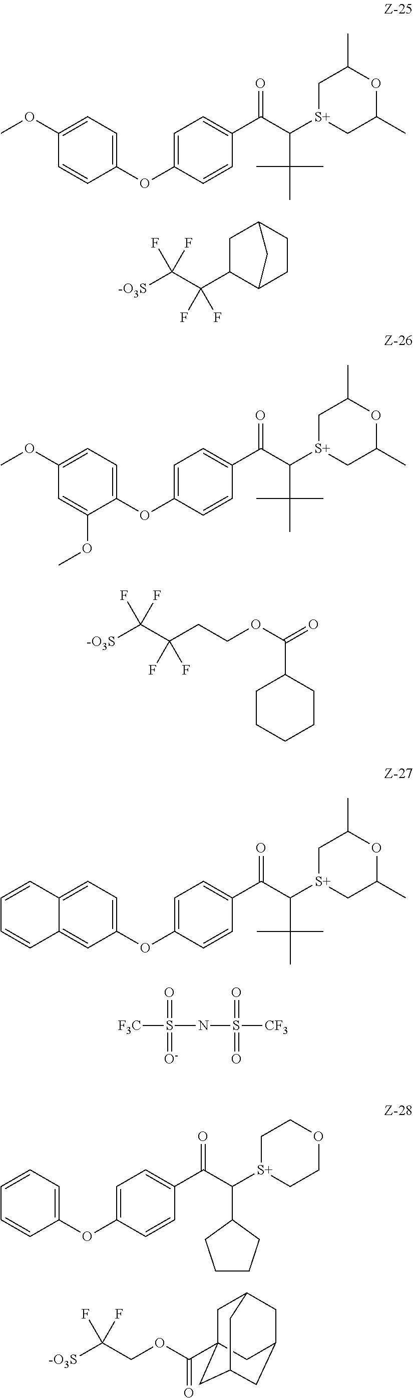

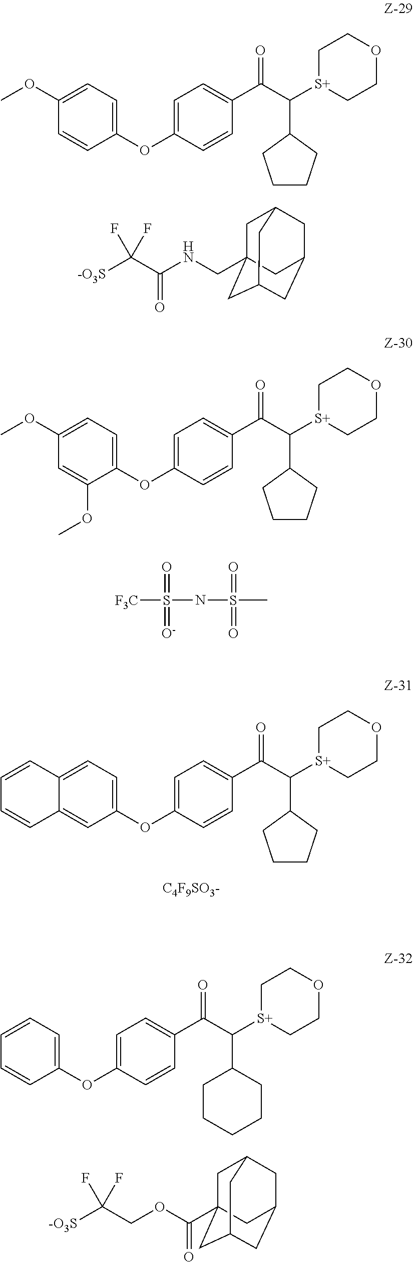

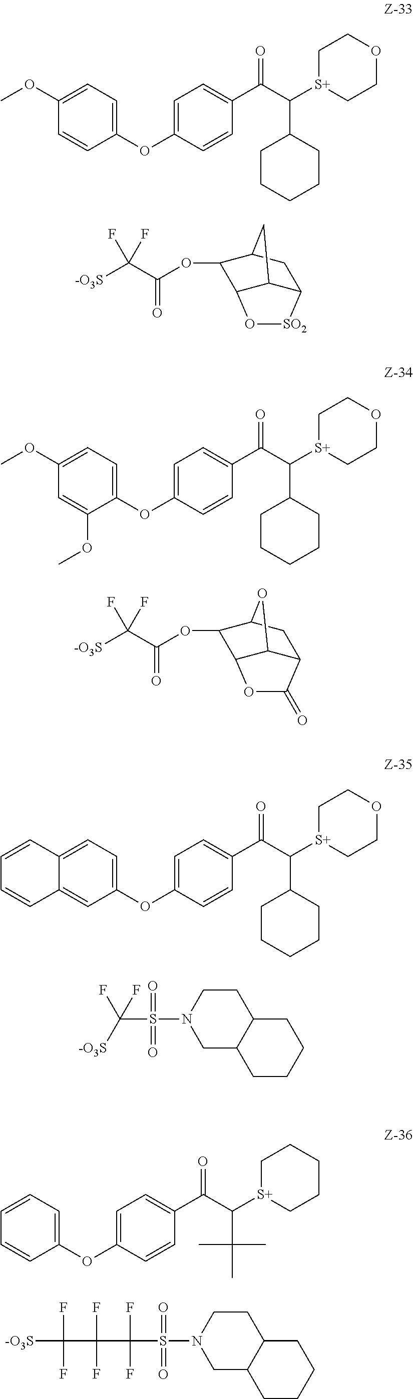

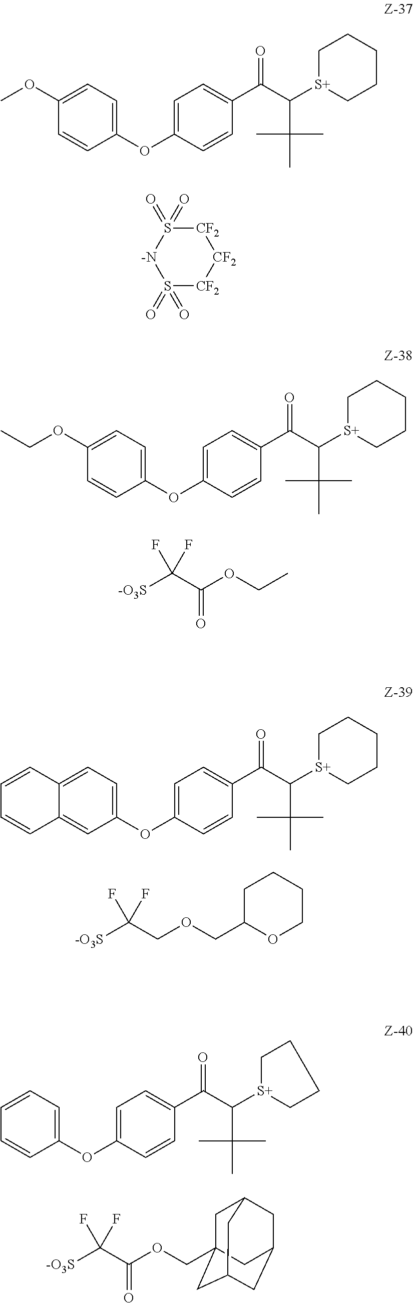

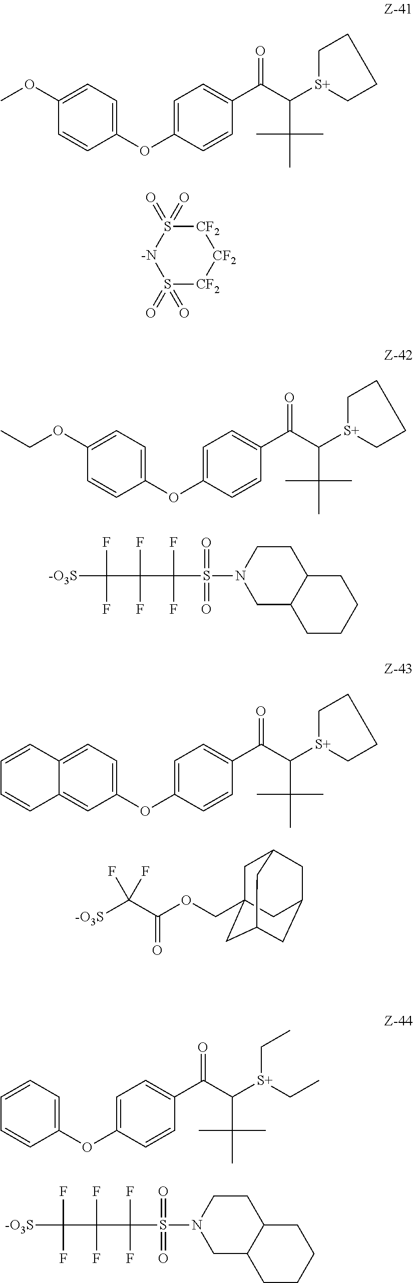

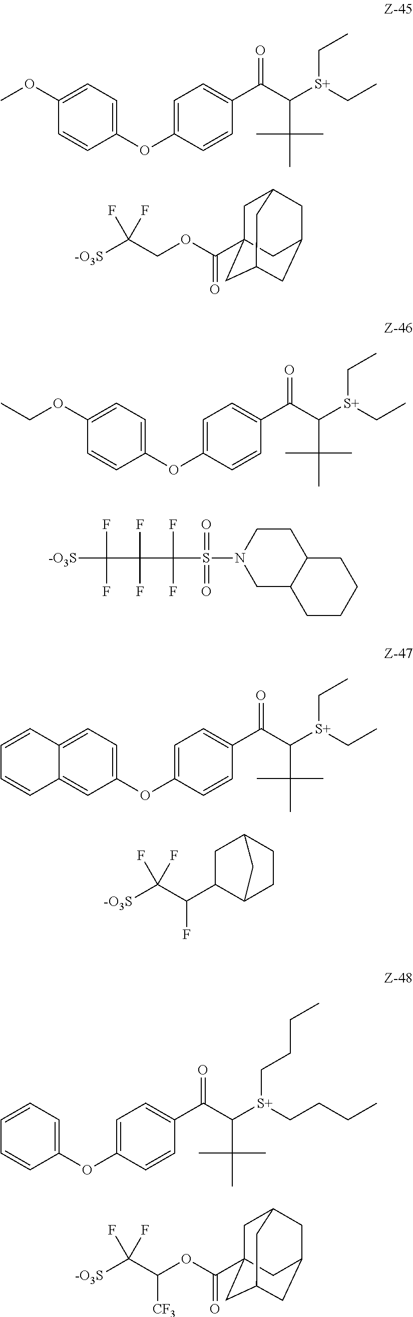

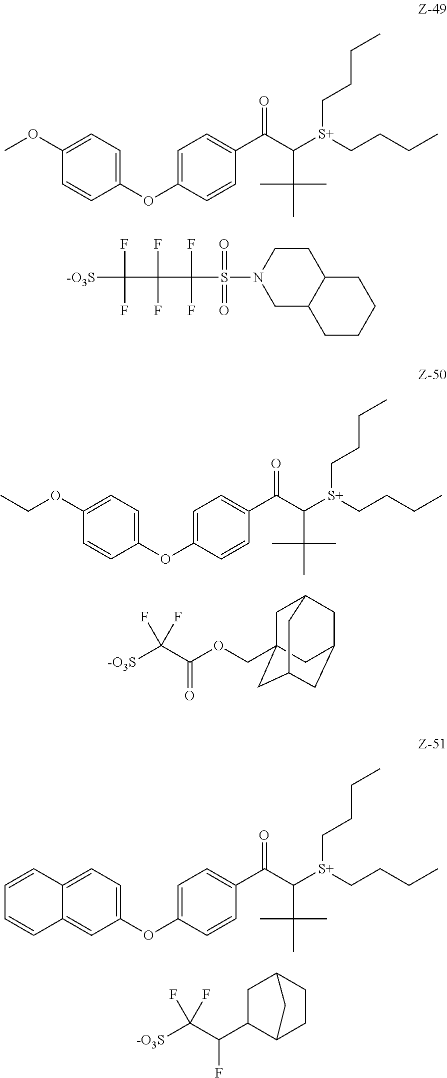

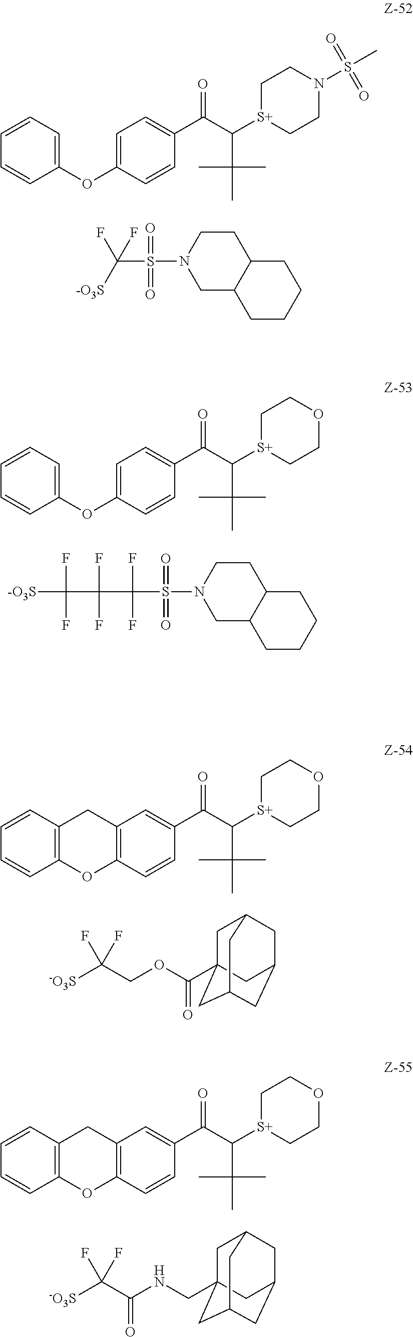

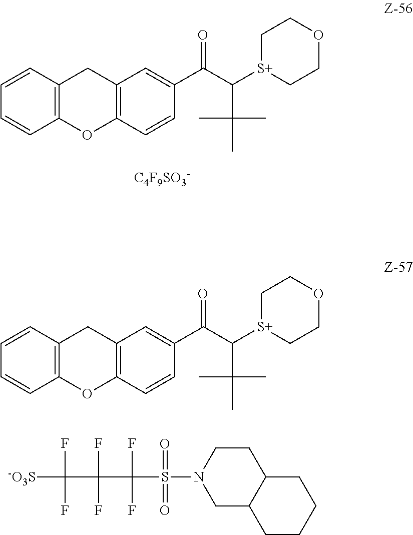

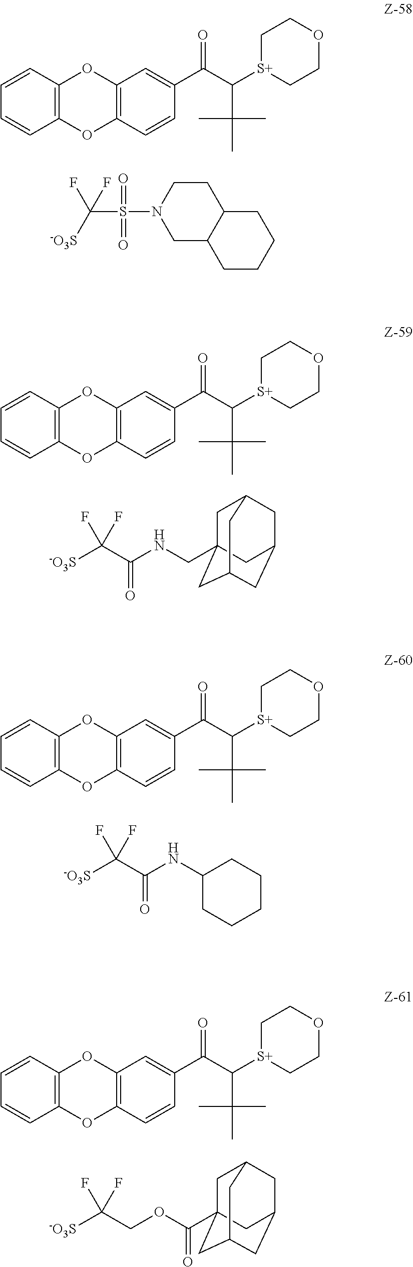

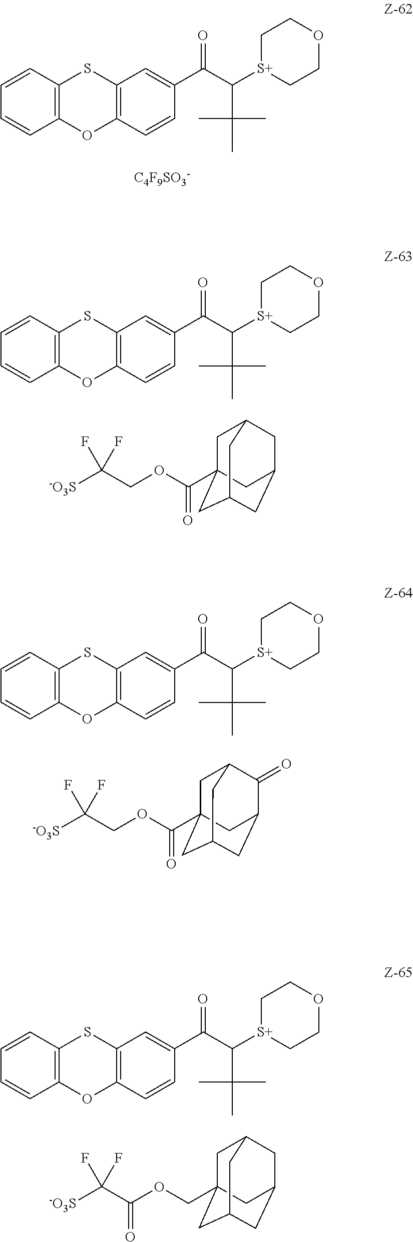

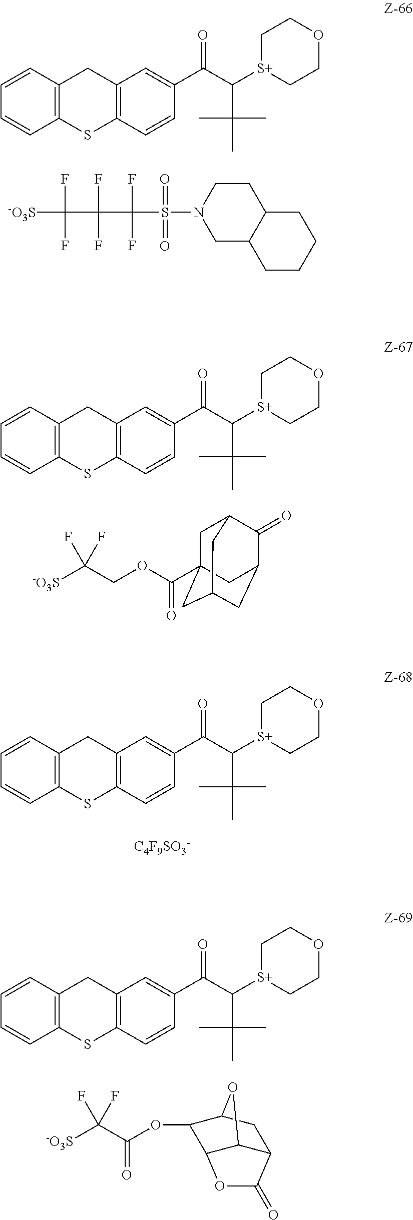

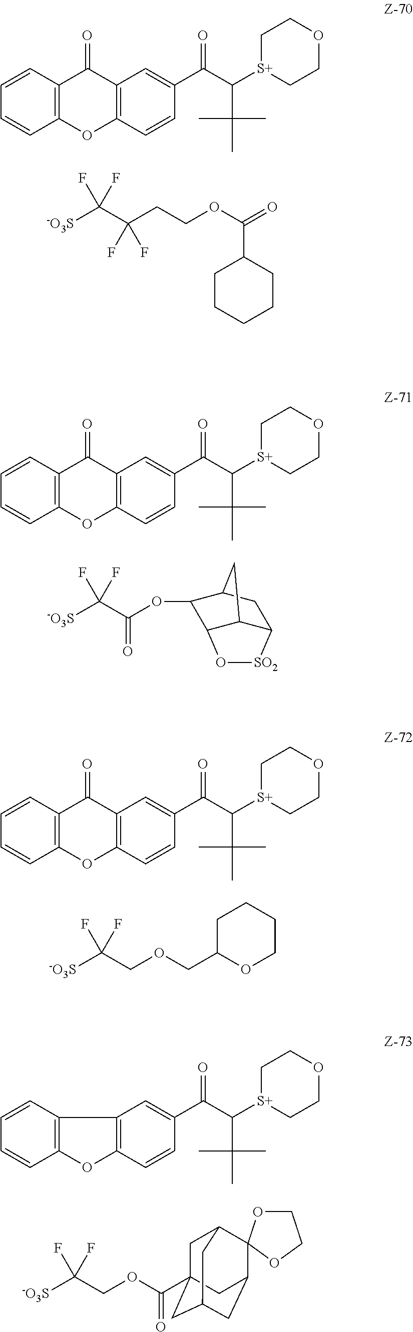

Description will be given of a method for synthesizing the compound (A).

It is possible to use sulfonic acid anions or salts thereof in General Formula (1) in the synthesis of the compound (A) which is represented by General Formula (1). It is possible to synthesize the sulfonic acid anions or salts thereof (for example, onium salt or a metal salt) in General Formula (1) which is used in the synthesis of the compound (A) using a general sulfonic acid esterification reaction or a sulfone amidating reaction. For example, it is possible to obtain the above by a method in which, after forming a sulfonamide bond, a sulfonic acid ester bond, or a sulfonimide bond by selectively reacting one sulfonylhalide part of a bissulfonylhalide compound with an amine, alcohol, an amide compound, or the like, the other sulfonylhalide portion is hydrolyzed, or a method which opens a ring of sulfonic acid anhydride using an amine, alcohol, or an amide compound.

Examples of salts of the sulfonic acid anion in General Formula (1) include sulfonic acid metal salt, sulfonic acid onium salt, and the like. Examples of metals in sulfonic acid metal salts include Na⁺, Li⁺, K⁺, and the like. Examples of onium cations in sulfonic acid onium salt include ammonium cations, sulfonium cations, iodonium cations, phosphonium cations, diazonium cations, and the like.

The compound (A) is able to synthesize the sulfonic acid anion which is represented by General Formula (1) described above by a method which carries out salt replacement with photoactive onium salt such as sulfonium salt which is equivalent to the sulfonium cation in General Formula (1) described above.

In the actinic-ray-sensitive or radiation-sensitive resin composition according to the present invention, it is possible to use the compound (A) as one type individually or in a combination of two or more types. The content ratio of the compound (A) in the composition of the present invention is preferably 0.1 mass % to 40 mass %, more preferably 1 mass % to 30 mass %, and even more preferably 10 mass % to 25 mass % using the total solid content of the composition as a reference.

In addition, the compound (A) may be used in a combination with an acid generating agent other than the compound (A) (also referred to below as a compound (A′) or an acid generating agent (A′)).

The compound (A′) is not particularly limited; however, preferable examples thereof include the compounds which are represented by General Formulae (ZI′), (ZII′), and (ZIII′) below.

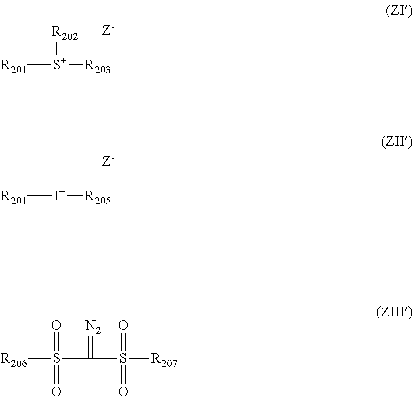

In General Formula (ZI′) described above, R₂₀₁, R₂₀₂, and R₂₀₃ each independently represents an organic group.

The number of carbon atoms of the organic group as R₂₀₁, R₂₀₂, and R₂₀₃ is generally 1 to 30 and preferably 1 to 20.

In addition, two out of R₂₀₁ to R₂₀₃ may form a ring structure by bonding with each other and an oxygen atom, a sulfur atom, an ester bond, an amide bond, and a carbonyl group may be included in the ring. Examples of a group formed by two out of R₂₀₁ to R₂₀₃ bonding with each other include an alkylene group (for example, a butylene group and a pentylene group).

Examples of the organic group which is represented by R₂₀₁, R₂₀₂, and R₂₀₃ include a corresponding group in a compound (ZI′-1) which will be described below.

Here, the compound (A) may be a compound which has a plurality of structures which are represented by General Formula (ZI′). For example, the compound may be a compound which has a structure where at least one of R₂₀₁ to R₂₀₃ of a compound which is represented by General Formula (ZI′) is bonded with at least another one of R₂₀₁ to R₂₀₃ of the compound which is represented by General Formula (ZI′) via a single bond or a linking group.

Z⁻ represents a non-nucleophilic anion (an anion which has a remarkably low ability to cause a nucleophilic reaction).

Examples of Z⁻ include sulfonic acid anions (aliphatic sulfonic acid anions, aromatic sulfonic acid anions, camphor sulfonic acid anions, and the like), carboxylate anions (aliphatic carboxylate anions, aromatic carboxylate anions, aralkyl carboxylate anions, and the like), sulfonylimide anions, bis(alkylsulfonyl) imide anions, tris(alkylsulfonyl) methide anions, and the like.

An aliphatic site in an aliphatic sulfonic acid anion and an aliphatic carboxylate anion may be an alkyl group or a cycloalkyl group and preferable examples thereof include a linear or branched alkyl group with 1 to 30 carbon atoms or a cycloalkyl group with 3 to 30 carbon atoms.

An aromatic group in an aromatic sulfonic acid anion and an aromatic carboxylate anion is preferably an aryl group with 6 to 14 carbon atoms and examples thereof include a phenyl group, a tolyl group, a naphthyl group, and the like.

An alkyl group, a cycloalkyl group, and an aryl group in the non-nucleophilic anion described above may have a substituent group. Specific examples of the substituent group include a nitro group, a halogen atom such as a fluorine atom, a carboxyl group, a hydroxyl group, an amino group, a cyano group, an alkoxy group (preferably with 1 to 15 carbon atoms), a cycloalkyl group (preferably with 3 to 15 carbon atoms), an aryl group (preferably with 6 to 14 carbon atoms), an alkoxycarbonyl group (preferably with 2 to 7 carbon atoms), an acyl group (preferably with 2 to 12 carbon atoms), an alkoxycarbonyloxy group (preferably with 2 to 7 carbon atoms), an alkylthio group (preferably with 1 to 15 carbon atoms), an alkylsulfonyl group (preferably with 1 to 15 carbon atoms), an alkyliminosulfonyl group (preferably with 2 to 15 carbon atoms), an aryloxysulfonyl group (preferably with 6 to 20 carbon atoms), an alkylaryloxysulfonyl group (preferably with 7 to 20 carbon atoms), a cycloalkylaryloxysulfonyl group (preferably with 10 to 20 carbon atoms), an alkyloxyalkyloxy group (preferably with 5 to 20 carbon atoms), a cycloalkylalkyloxyalkyloxy group (preferably with 8 to 20 carbon atoms), and the like. Examples of an aryl group and a ring structure of each group further include an alkyl group (preferably with 1 to 15 carbon atoms) as a substituent group.

An aralkyl group in an aralkyl carboxylate anion is preferably an aralkyl group with 7 to 12 carbon atoms and examples thereof include a benzyl group, a phenethyl group, a naphthylmethyl group, a naphthylethyl group, a naphthylbutyl group, and the like.

Examples of sulfonylimide anions include saccharin anions.

An alkyl group in a bis(alkylsulfonyl) imide anion and a tris(alkylsulfonyl) methide anion is preferably an alkyl group with 1 to 5 carbon atoms.

Two alkyl groups in a bis(alkylsulfonyl) imide anion may form an alkylene group (preferably with 2 to 4 carbon atoms) by linking with each other and may form a ring with an imide group and two sulfonyl groups.

Examples of a substituent group which an alkylene group formed by the alkyl group and two alkyl groups in bis(alkylsulfonyl) imide anion linking with each other may have include a halogen atom, an alkyl group which is substituted with a halogen atom, an alkoxy group, an alkylthio group, an alkyloxysulfonyl group, an aryloxysulfonyl group, a cycloalkylaryloxysulfonyl group, and the like, and a fluorine atom or an alkyl group which is substituted with a fluorine atom is preferable.

Other examples of Z⁻ include fluorinated phosphorus (for example, PF₆⁻), fluorinated boron (for example, BF₄⁻), fluorinated antimony (for example, SbF₆⁻), and the like. As Z⁻, an aliphatic sulfonic acid anion where at least a position of sulfonic acid is substituted with a fluorine atom, an aromatic sulfonic acid anion which is substituted with a fluorine atom or a group which has a fluorine atom, a bis(alkylsulfonyl) imide anion where an alkyl group is substituted with a fluorine atom, and a tris(alkylsulfonyl) methimide anion where an alkyl group is substituted with a fluorine atom are preferable. The non-nucleophilic anion is more preferably a perfluoro aliphatic sulfonic acid anion (even more preferably with 4 to 8 carbon atoms), or a benzene sulfonic acid anion which has a fluorine atom, even more preferably a nonafluorobutan sulfonic acid anion, a perfluorooctan sulfonic acid anion, a pentafluorobenzene sulfonic acid anion, or a 3,5-bis(trifluoromethyl)benzene sulfonic acid anion.

From the point of view of the acid strength, it is preferable that the pKa of generated acid is −1 or less in order to improve the sensitivity.

Examples of even more preferable (ZI′) components include a compound (ZI′-1) which will be described below.

The compound (ZI′-1) is an arylsulfonium compound where at least one of R₂₀₁ to R₂₀₃ of General Formula (ZI′) described above is an aryl group, that is, a compound which has arylsulfonium as cations.

With regard to the arylsulfonium compound, all of R₂₀₁ to R₂₀₃ may be aryl groups and a part of R₂₀₁ to R₂₀₃ may be an aryl group and the rest an alkyl group or a cycloalkyl group; however, it is preferable that all of R₂₀₁ to R₂₀₃ are aryl groups.

Examples of the arylsulfonium compound include a triarylsulfonium compound, a diarylalkylsulfonium compound, an aryldialkylsulfonium compound, a diarylcycloalkylsulfonium compound, and an aryldicycloalkylsulfonium compound, and a triarylsulfonium compound is preferable.

An aryl group of an arylsulfonium compound is preferably a phenyl group or a naphthyl group, and a phenyl group is more preferable. The aryl group may be an aryl group which has a heterocyclic structure which has an oxygen atom, a nitrogen atom, a sulfur atom, or the like. Examples of the heterocyclic structure include a pyrrole residue, a furan residue, a thiophene residue, an indole residue, a benzofuran residue, a benzothiophene residue, and the like. In a case where the arylsulfonium compound has two or more aryl groups, the two or more aryl groups may be the same or may be different.

An alkyl group or a cycloalkyl group which the arylsulfonium compound has as necessary is preferably a linear or branched alkyl group with 1 to 15 carbon atoms and a cycloalkyl group with 3 to 15 carbon atoms and examples thereof include a methyl group, an ethyl group, a propyl group, an n-butyl group, a sec-butyl group, a t-butyl group, a cyclopropyl group, a cyclobutyl group, a cyclohexyl group, and the like.

An aryl group, an alkyl group, and a cycloalkyl group of R₂₀₁ to R₂₀₃ may have an alkyl group (for example, with 1 to 15 carbon atoms), a cycloalkyl group (for example, 3 to 15 carbon atoms), an aryl group (for example, with 6 to 14 carbon atoms), an alkoxy group (for example, with 1 to 15 carbon atoms), a halogen atom, a hydroxyl group, and a phenylthio group as a substituent group. Preferable substituent groups are a linear or branched alkyl group with 1 to 12 carbon atoms, a cycloalkyl group with 3 to 12 carbon atoms, and a linear, branched, or cyclic alkoxy group with 1 to 12 carbon atoms, and an alkyl group with 1 to 4 carbon atoms, and an alkoxy group with 1 to 4 carbon atoms are more preferable. The substituent group may be substituted with any one of three R₂₀, to R₂₀₃ or may be substituted with all of the three. In addition, in a case where R₂₀₁ to R₂₀₃ is an aryl group, it is preferable that the substituent group is substituted at the p-position of the aryl group.

Next, description will be given of General Formulae (ZII′) and (ZIII′).

In General Formulae (ZII′) and (ZIII′), R₂₀₄ to R₂₀₇ each independently represents an aryl group, an alkyl group or a cycloalkyl group.

The aryl group, the alkyl group, and the cycloalkyl group of R₂₀₄ to R₂₀₇ are the same as the aryl group which is described as the aryl group, the alkyl group, and the cycloalkyl group of R₂₀₁ to R₂₀₃ in the compound (ZI′-1) described above.

The aryl group, the alkyl group, and the cycloalkyl group of R₂₀₄ to R₂₀₇ may have a substituent group. Examples of the substituent group also include the substituent groups which the aryl group, the alkyl group, and the cycloalkyl group of R₂₀₁ to R₂₀₃ in the compound (ZI′-1) described above may have.

Z⁻ represents a non-nucleophilic anion and examples thereof include the same non-nucleophilic anions as the non-nucleophilic anions of Z⁻ in General Formula (ZI′).

Examples of an acid generating agent (A′) which may be used together with the acid generating agent in the present invention also further include compounds which are represented by General Formulae (ZIV′), (ZV′), and (ZVI′) below.

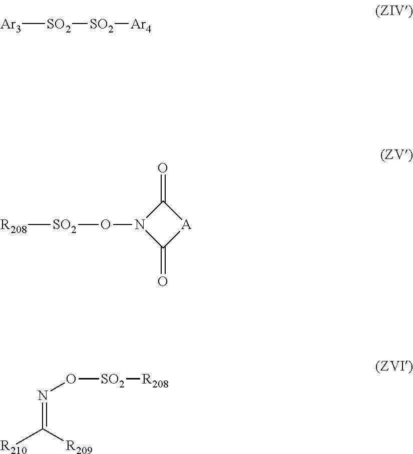

In General Formulae (ZIV′) to (ZVI′), Ar₃ and Ar₄ each independently represents an aryl group.

R₂₀₈, R₂₀₉, and R₂₁₀ each independently represents an alkyl group, a cycloalkyl group, or an aryl group.

A represents an alkylene group, an alkenylene group, or an arylene group.

Specific examples of the aryl group of Arₐ, Ar₄, R₂₀₈, R₂₀₉, and R₂₁₀ include the same aryl groups as the specific examples of the aryl group as R₂₀₁, R₂₀₂, and R₂₀₃ in General Formula (ZI′-1) described above.

Specific examples of the alkyl group and the cycloalkyl group of R₂₀₈, R₂₀₉, and R₂₁₀ each include the same examples as the specific examples of the alkyl group and the cycloalkyl group as R₂₀₁, R₂₀₂, and R₂₀₃ in General Formula (ZI′-1) described above.

Examples of the alkylene group of A include an alkylene group with 1 to 12 carbon atoms (for example, a methylene group, an ethylene group, a propylene group, an isopropylene group, a butylene group, an isobutylene group, and the like), examples of an alkenylene group of A include an alkenylene group with 2 to 12 carbon atoms (for example, an ethenylene group, a propenylene group, a butenylene group, and the like), and examples of an arylene group of A include an arylene group with 6 to 10 carbon atoms (for example, a phenylene group, a tolylene group, a naphthylene group, and the like).

Particularly preferable examples will be given below from among acid generating agents which may be used together with the acid generating agent of the present invention.

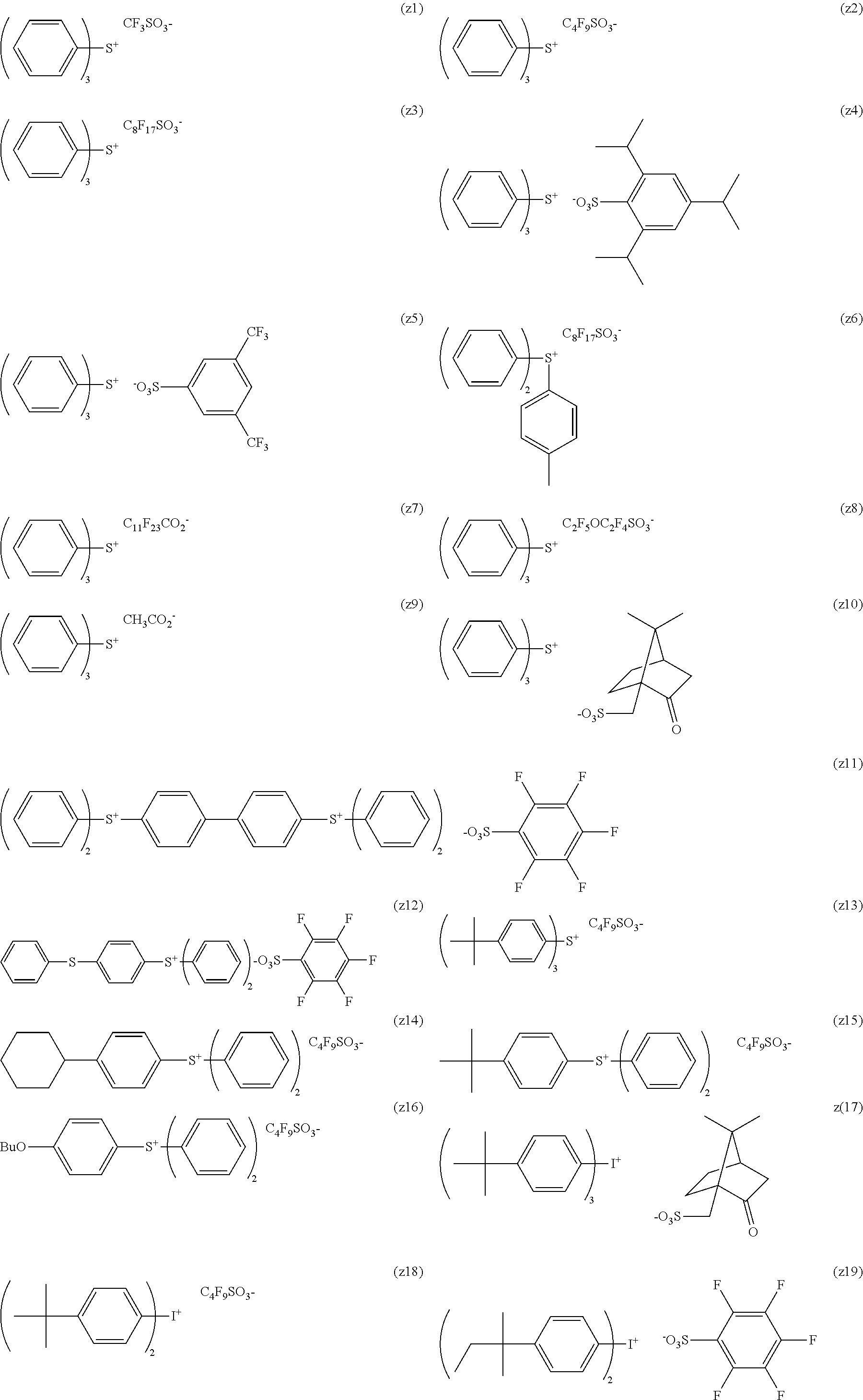

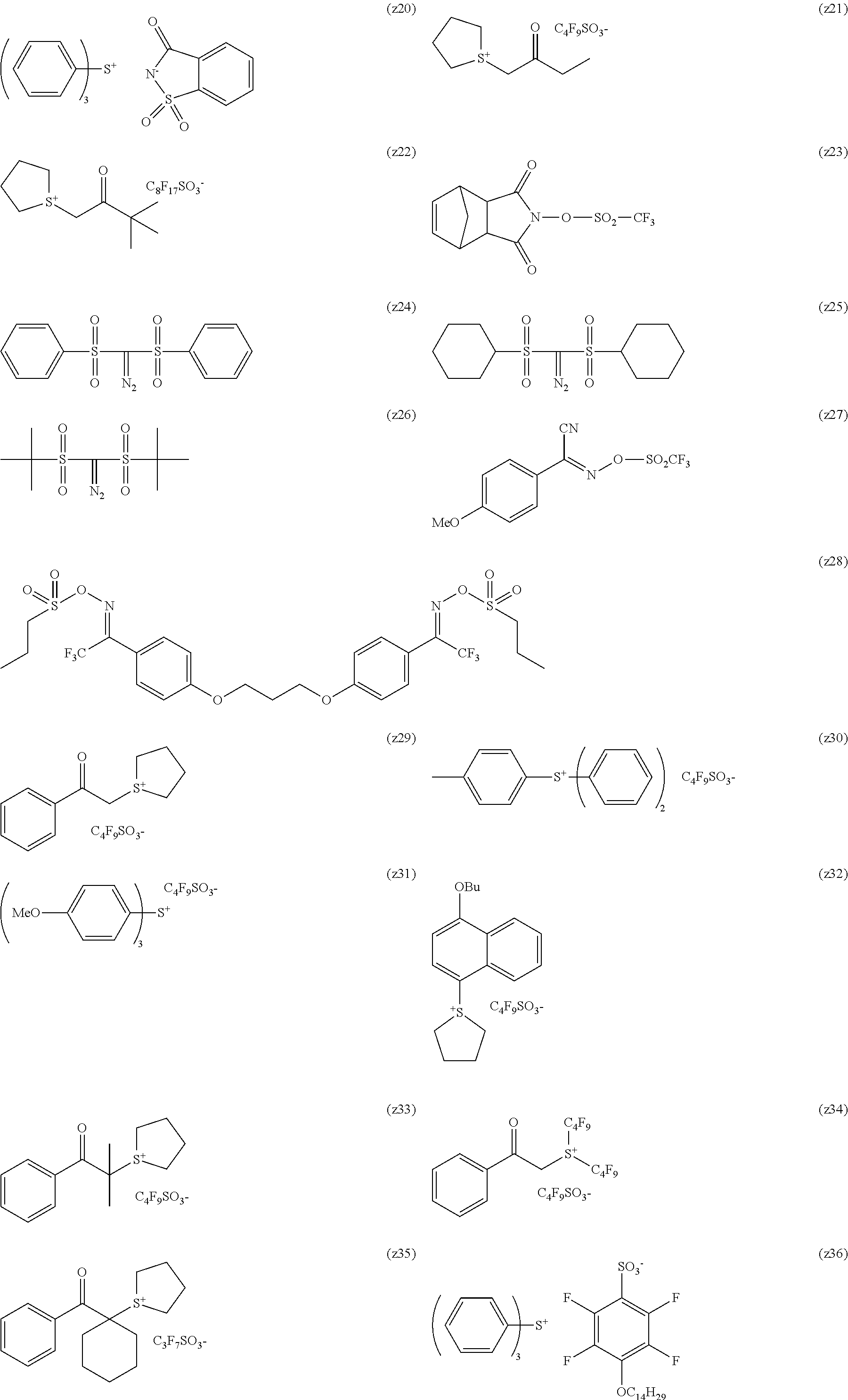

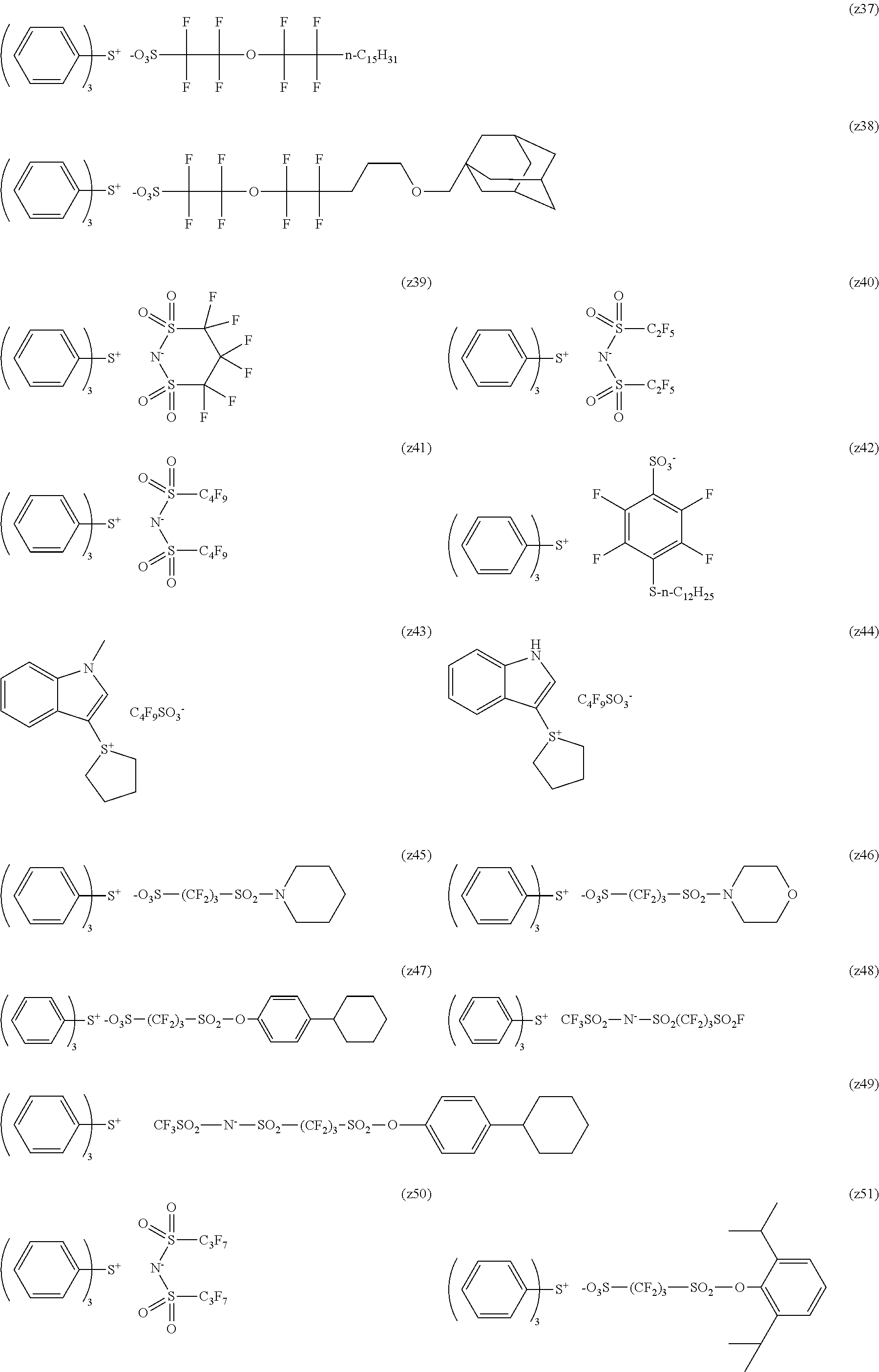

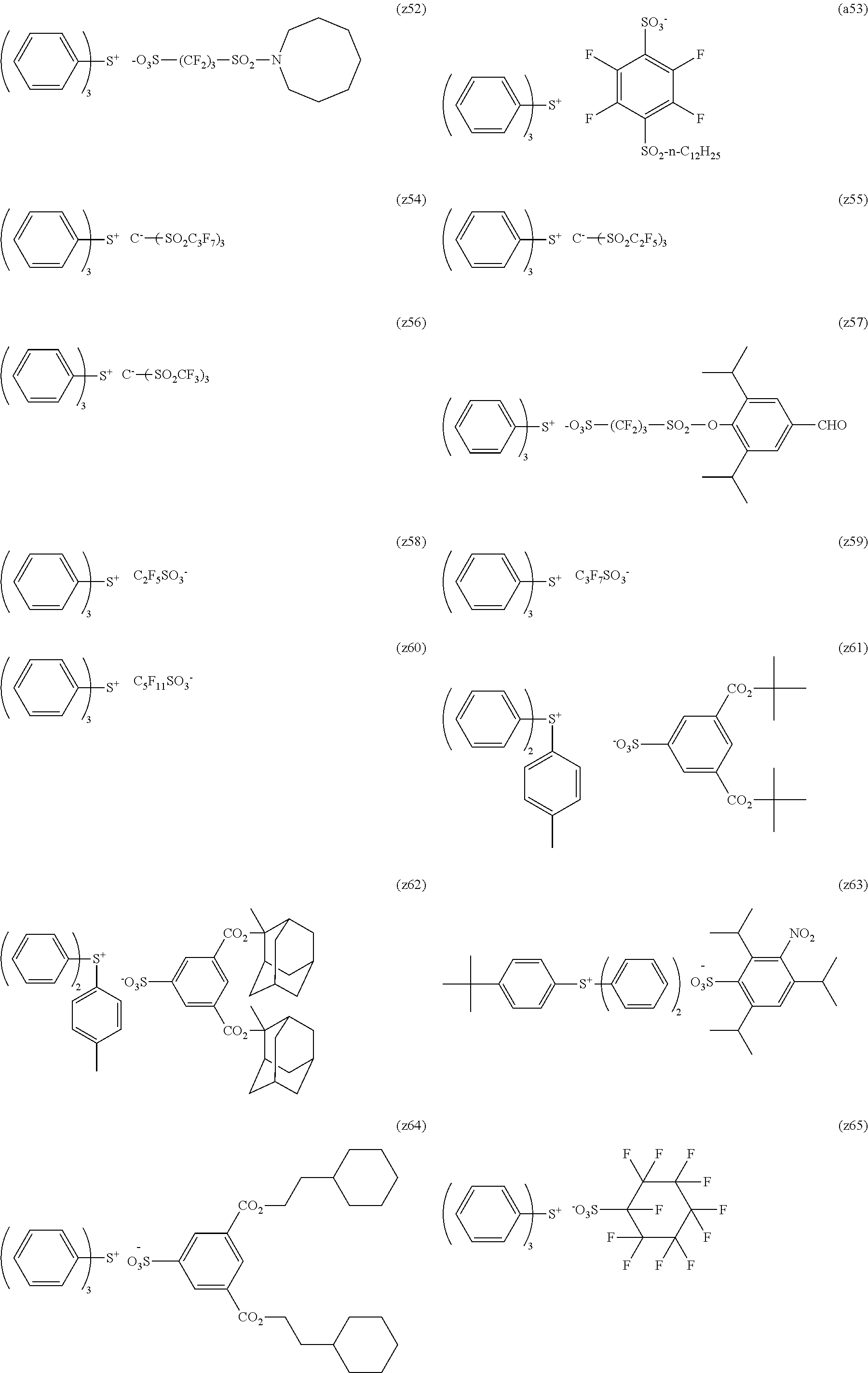

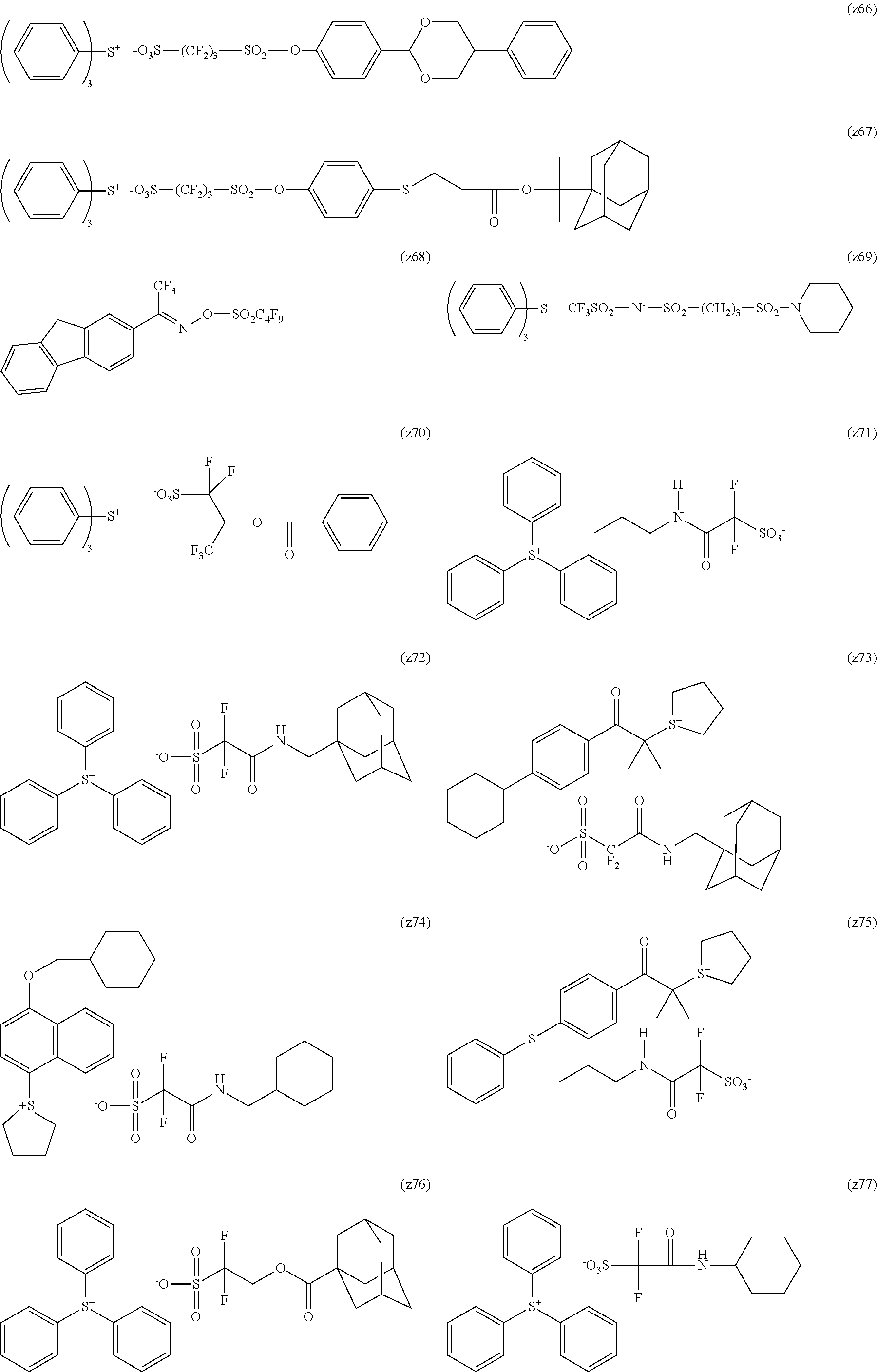

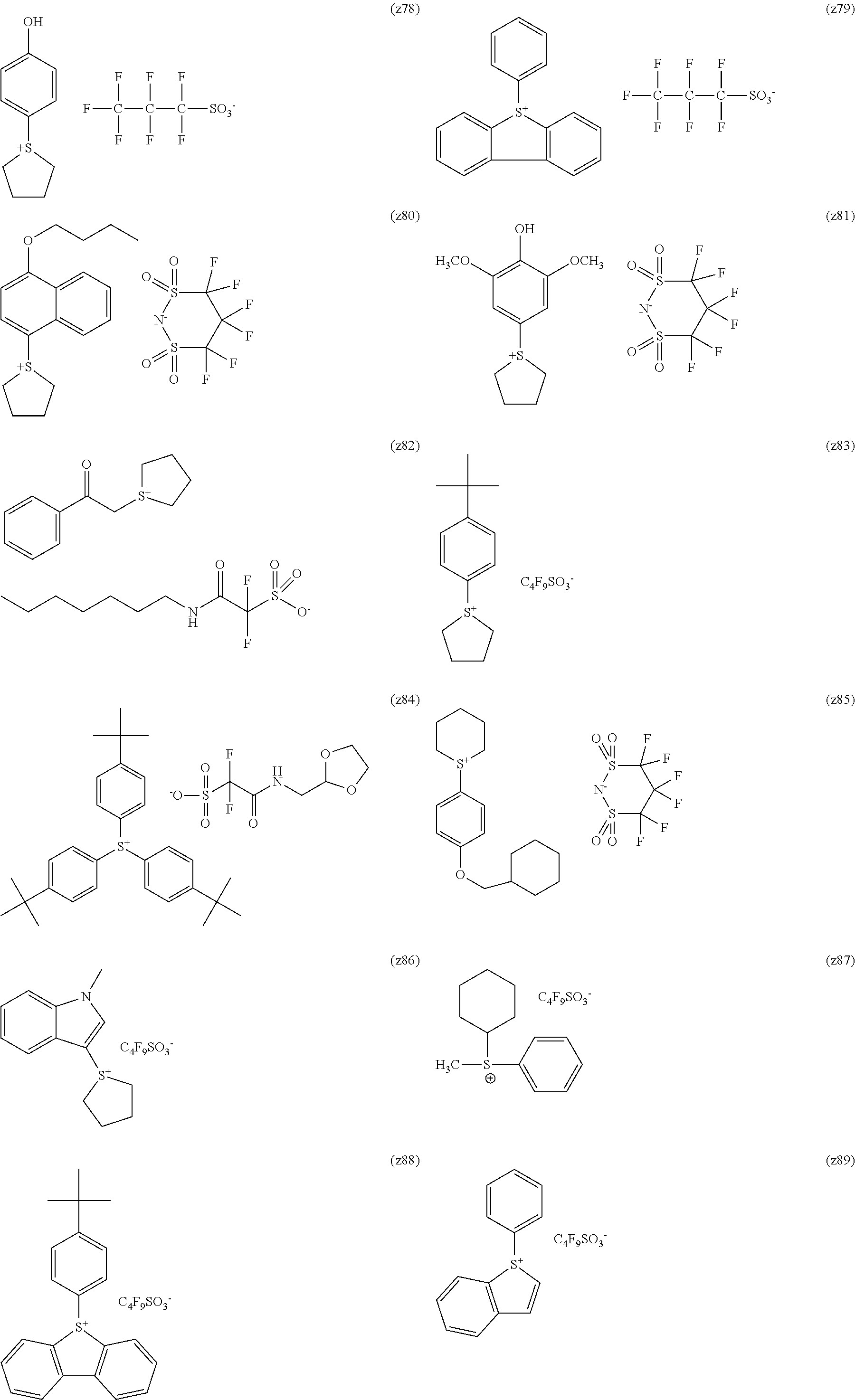

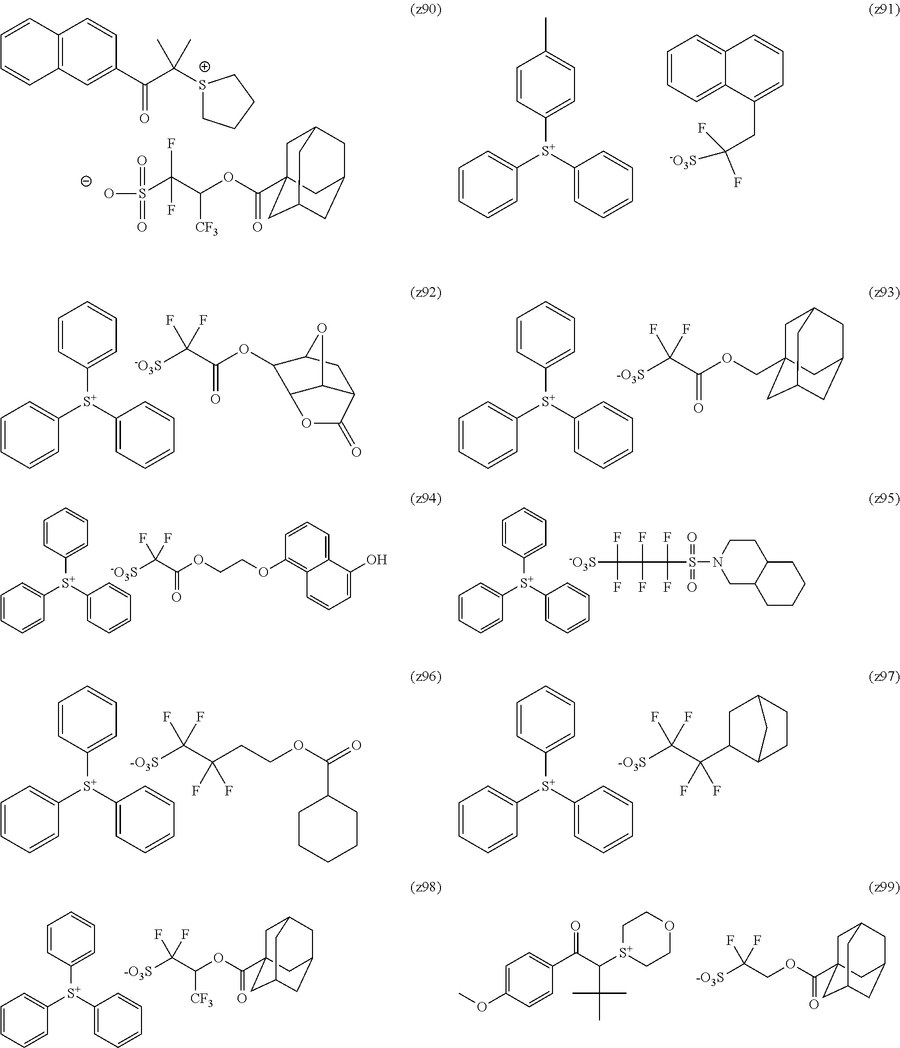

The usage amount of the acid generating agent in a case of using both the compound (A) and the compound (A′) is preferably 99/1 to 20/80, more preferably 99/1 to 40/60, and even more preferably 99/1 to 50/50 in terms of the mass ratio (compound (A)/compound (A′)). In addition, in a case of using both the compound (A) and the compound (A′), a combination where the anion portion of the compound (A) and the anion portion of the compound (A′) are the same is preferable.

[2] Resin Decomposed by an Action of an Acid and Having an Increased Solubility with Respect to an Alkaline Developing Solution

The actinic-ray-sensitive or radiation-sensitive resin composition of the present invention may contain a resin which is decomposed by an action of an acid and which has increased solubility with respect to an alkaline developing solution (also referred to below as an “acid-decomposable resin” or “resin (B)”).

The acid-decomposable resin has a group (also referred to below as an “acid-decomposable group”) which is decomposed by an action of an acid and which generates an alkali-soluble group, on a main chain or a side chain of the resin or on both the main chain and the side chain.

The resin (B) is preferably insoluble or sparingly soluble to an alkali developing solution.

The acid-decomposable group preferably has a structure which is protected by a group where an alkali-soluble group is decomposed and leaves by an action of an acid.

Examples of the alkali-soluble group include a phenolic hydroxyl group, a carboxyl group, a fluorinated alcohol group, a sulfonic acid group, a sulfonamide group, a sulfonylimide group, an (alkylsulfonyl) (alkylcarbonyl)methylene group, an (alkylsulfonyl) (alkylcarbonyl) imide group, a bis(alkylcarbonyl)methylene group, a bis(alkylcarbonyl) imide group, a bis(alkylsulfonyl)methylene group, a bis(alkylsulfonyl) imide group, a tris(alkylcarbonyl)methylene group, a tris(alkylsulfonyl)methylene group, and the like.

Examples of a preferable alkali-soluble group include a carboxyl group, a fluorinated alcohol group (preferably a hexafuluoro isopropanol group), and a sulfonic acid group.

A preferable group as the acid-decomposable group is a group where hydrogen atoms of the alkali-soluble groups are substituted with a group which is made to leave by acid.

Examples of the group which is made to leave by acid include —C(R₃₆)(R₃₇)(R₃₈), —C(R₃₆)(R₃₇)(OR₃₉), —C(R₀₁)(R₀₂)(OR₃₉), and the like.

In the formula, R₃₆ to R₃₉ each independently represents an alkyl group, a cycloalkyl group, an aryl group, an aralkyl group, or an alkenyl group. R₃₆ and R₃₇ may form a ring by bonding with each other.

R₀₁ and R₀₂ each independently represents a hydrogen atom, an alkyl group, a cycloalkyl group, an aryl group, an aralkyl group, or an alkenyl group.

The acid-decomposable group is preferably a cumyl ester group, an enol ester group, an acetal ester group, a tertiary alkyl ester group, and the like. The tertiary alkyl ester group is more preferable.

As a repeating unit which the resin (B) may contain and which has an acid-decomposable group, a repeating unit which is represented by General Formula (AI) below is preferable.

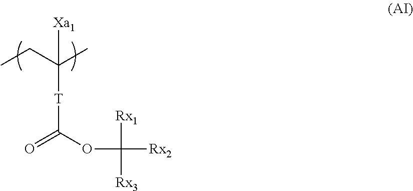

In General Formula (AI), Xa₁ represents an alkyl group which may have a hydrogen atom and a substituent group.

T represents a single bond or a divalent bonding group.

Rx₁ to Rx₃ each independently represents an alkyl group (linear or branched) or a cycloalkyl group (monocyclic or polycyclic).

Two of Rx₁ to Rx₃ may be bonded to form a cycloalkyl group (monocyclic or polycyclic).

Examples of an alkyl group which is represented by Xa₁ and which may have a substituent group include a methyl group or a group which is represented by —CH₂—R₁₁. R₁₁ represents a halogen atom (a fluorine atom and the like), a hydroxyl group, or a monovalent organic group and examples thereof include an alkyl group with 5 or fewer carbon atoms and an acyl group with 5 or fewer carbon atoms, and an alkyl group with 3 or fewer carbon atoms is preferable and a methyl group is more preferable. In one aspect, Xa₁ is preferably a hydrogen atom, a methyl group, a trifluoromethyl group, a hydroxylmethyl group, or the like.

Examples of divalent bonding groups of T include an alkylene group, —COO-Rt- group, —O-Rt- group, and the like. Rt represents an alkylene group or a cycloalkylene group.

T is preferably a single bond or —COO-Rt- group. Rt is preferably an alkylene group with 1 to 5 carbon atoms and is more preferably a —CH₂— group, a —(CH₂)₂— group, and a —(CH₂)₃— group.

As alkyl groups of Rx₁ to Rx₃, an alkyl group with 1 to 4 carbon atoms such as a methyl group, an ethyl group, an n-propyl group, an isopropyl group, an n-butyl group, an isobutyl group, and a t-butyl group is preferable.

As cycloalkyl groups of Rx₁ to Rx₃, monocyclic cycloalkyl groups such as a cyclopentyl group and a cyclohexyl group, and polycyclic cycloalkyl groups such as a norbornyl group, a tetracyclodecanyl group, a tetracyclododecanyl group, and an adamantyl group are preferable.

As a cycloalkyl group which is formed by two of Rx₁ to Rx₃ bonding with each other, a monocyclic cycloalkyl group such as a cyclopentyl group or a cyclohexyl group, or polycyclic cycloalkyl groups such as a norbornyl group, a tetracyclodecanyl group, a tetracyclododecanyl group, and an adamantyl group are preferable. A monocyclic cycloalkyl group with 5 to 6 carbon atoms is particularly preferable.

With regard to the cycloalkyl group which is formed by two of Rx₁ to Rx₃ bonding with each other, for example, one of methylene groups which configure a ring may be substituted with a hetero atom such as an oxygen atom or a group which has a hetero atom such as a carbonyl group.

With regard to a repeating unit which is represented by General Formula (AI), for example, an aspect where Rx₁ is a methyl group or an ethyl group and the cycloalkyl group described above is formed by Rx₂ and Rx₃ bonding with each other is preferable.

Each of the groups described above may have a substituent group and examples of the substituent group include an alkyl group (with 1 to 4 carbon atoms), a halogen atom, a hydroxyl group, an alkoxy group (with 1 to 4 carbon atoms), a carboxyl group, an alkoxycarbonyl group (with 2 to 6 carbon atoms), and the like, and a group with 8 or fewer carbon atoms is preferable.

The total content of the repeating unit which has an acid-decomposable group is preferably 20 mol % to 80 mol %, more preferably 25 mol % to 75 mol %, and even more preferably 30 mol % to 70 mol % with respect to all the repeating units in the resin (B).

In detail, it is possible to use the specific examples disclosed in paragraph 0265 of US2012/0135348A1; however, the present invention is not limited thereto.

The resin (B) preferably contains, for example, a repeating unit which is represented by General Formula (3) as the repeating unit which is represented by General Formula (AI).

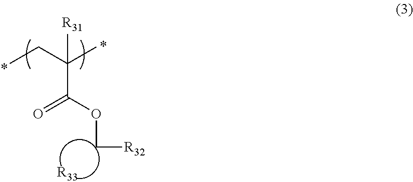

In General Formula (3), R₃₁ represents a hydrogen atom or an alkyl group.

R₃₂ represents a methyl group, an ethyl group, an n-propyl group, an isopropyl group, an n-butyl group, an isobutyl group, or a sec-butyl group.

R₃₃ represents an atom group which is necessary for forming a monocyclic alicyclic hydrocarbon structure with the carbon atom bonded with R₃₂. With regard to the alicyclic hydrocarbon structure, a part of the carbon atoms which configure a ring may be substituted with hetero atoms or a group which has hetero atoms.

The alkyl group of R₃₁ may have a substituent group and examples of the substituent group include a fluorine atom, a hydroxyl group, and the like.

R₃₁ preferably represents a hydrogen atom, a methyl group, a trifluoromethyl group, or a hydroxymethyl group.

R₃₂ is preferably a methyl group, an ethyl group, an n-propyl group, or an isopropyl group, and more preferably a methyl group or an ethyl group.

A monocyclic alicyclic hydrocarbon structure formed by R₃₃ with a carbon atom preferably has 3 to 8 ring members and more preferably has 5 or 6 ring members.

In the monocyclic alicyclic hydrocarbon structure formed by R₃₃ with a carbon atom, examples of hetero atoms which may configure the ring include an oxygen atom, a sulfur atom, and the like and examples of the group which has a hetero atom include a carbonyl group and the like. However, it is preferable that the group which has a hetero atom is not an ester group (an ester bond).

The monocyclic alicyclic hydrocarbon structure formed by R₃₃ with a carbon atom is preferably formed only by carbon atoms and hydrogen atoms.

Specific examples of a repeating unit which has a structure which is represented by General Formula (3) include the repeating unit below; however, the present invention is not limited thereto.

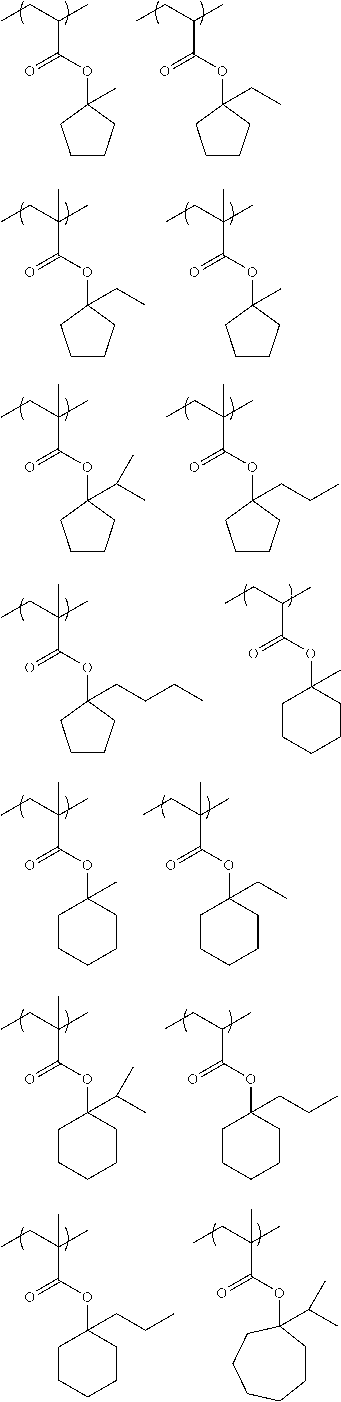

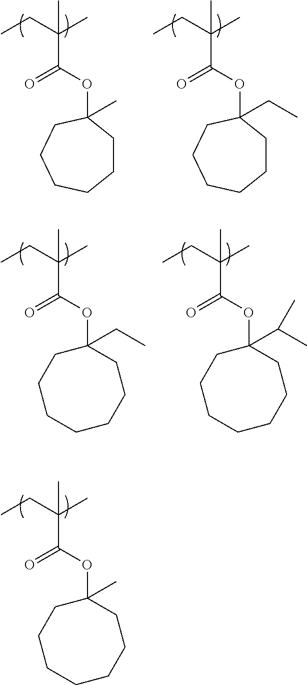

The content of the repeating unit which has a structure which is represented by General Formula (3) is preferably 20 mol % to 80 mol %, more preferably 25 mol % to 75 mol %, and even more preferably 30 mol % to 70 mol % with respect to all the repeating units in the resin (B).

More preferably, the resin (B) is a resin which, for example, has at least any one of a repeating unit which is represented by General Formula (I) and a repeating unit which is represented by General Formula (II) as a repeating unit which is represented by General Formula (AI).

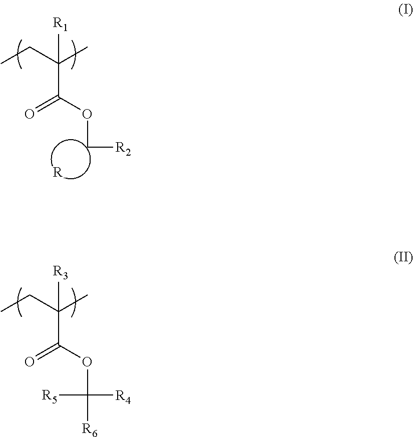

In the Formulae (I) and (II), R₁ and R₃ each independently represents a hydrogen atom, a methyl group which may have a substituent group, or a group which is represented by —CH₂—R₁₁. R₁₁ represents a monovalent organic group.

R₂, R₄, R₅, and R₆ each independently represents an alkyl group or a cycloalkyl group.

R represents an atom group which is necessary for forming an alicyclic structure with a carbon atom bonded with R₂.

R₁ and R₃ preferably represent a hydrogen atom, a methyl group, a trifluoromethyl group, or a hydroxymethyl group. Specific examples and preferable examples of the monovalent organic group in R₁₁ are the same as described in R₁₁ in General Formula (AI).

The alkyl group in R₂ may be a linear type or a branched type and may have a substituent group.

The cycloalkyl group in R₂ may be monocyclic or polycyclic and may have a substituent group.

R₂ is preferably an alkyl group, more preferably an alkyl group with 1 to 10 carbon atoms, and even more preferably an alkyl group with 1 to 5 carbon atoms and examples thereof include a methyl group, an ethyl group, and the like.

R represents an atom group which is necessary for forming an alicyclic structure with a carbon atom. The alicyclic structure formed by R with the carbon atom is preferably a monovalent alicyclic structure and the number of carbon atoms is preferably 3 to 7 and more preferably 5 or 6.

R₃ is preferably a hydrogen atom or a methyl group, more preferably a methyl group.

The alkyl group in R₄, R₅, and R₆ may be a linear type or a branched type and may have a substituent group. As the alkyl group, an alkyl group with 1 to 4 carbon atoms such as a methyl group, an ethyl group, an n-propyl group, an isopropyl group, an n-butyl group, an isobutyl group, and a t-butyl group is preferable.

The cycloalkyl group in R₄, R₅, and R₆ may be monocyclic or polycyclic and may have a substituent group. As the cycloalkyl group, a monovalent cycloalkyl group such as a cyclopentyl group or a cyclohexyl group, and a polycyclic cycloalkyl group such as a norbornyl group, a tetracyclodecanyl group, a tetracyclododecanyl group, or an adamantyl group are preferable.

Examples of a substituent group which each of the groups described above may have include the same groups described above as substituent groups which each of the groups in General Formula (AI) may have.

More preferably, the acid-decomposable resin is a resin which includes a repeating unit which is represented by General Formula (I) and a repeating unit which is represented by General Formula (II) as a repeating unit which is represented by General Formula (AI).

In addition, in other embodiments, the acid-decomposable resin is more preferably a resin which includes at least two types of repeating units which are represented by General Formula (I) as the repeating units which are represented by General Formula (AI). In a case of including two or more types of repeating units of General Formula (I), it is preferable to include both of a repeating unit where the alicyclic structure formed by R with a carbon atom is a monocyclic alicyclic structure and a repeating unit where the alicyclic structure formed by R with a carbon atom is a polycyclic alicyclic structure. As the monovalent alicyclic structure, the number of carbon atoms is preferably 5 to 8, more preferably 5 or 6, and particularly preferably 5. As the polycyclic alicyclic structure, a norbornyl group, a tetracyclodecanyl group, a tetracyclododecanyl group, and an adamantyl group are preferable.

In addition, the resin (B) may have a repeating unit which is decomposed by an action of an acid and generates an alcoholic hydroxyl group as represented below as a repeating unit which has an acid-decomposable group.

In the specific example below, Xa₁ represents a hydrogen atom, CH₃, CF₃, or CH₂OH.

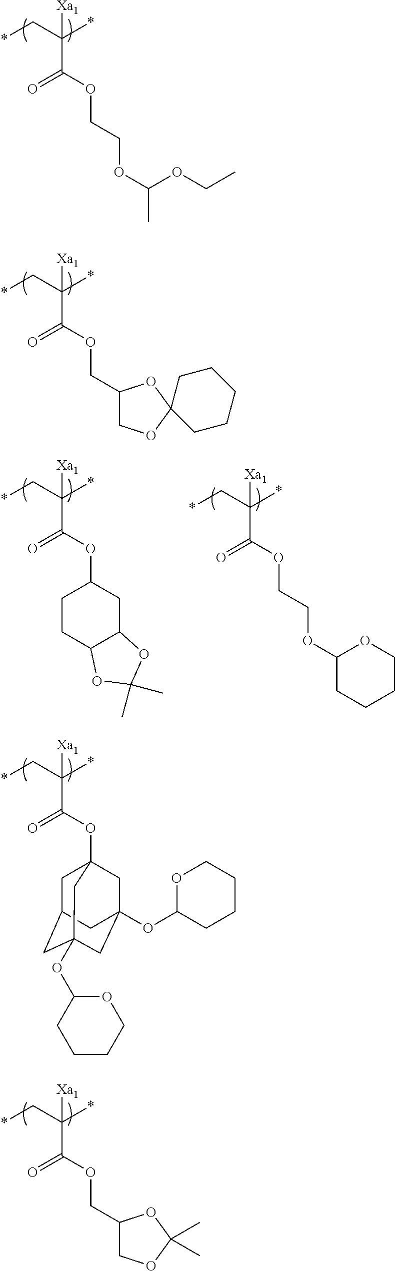

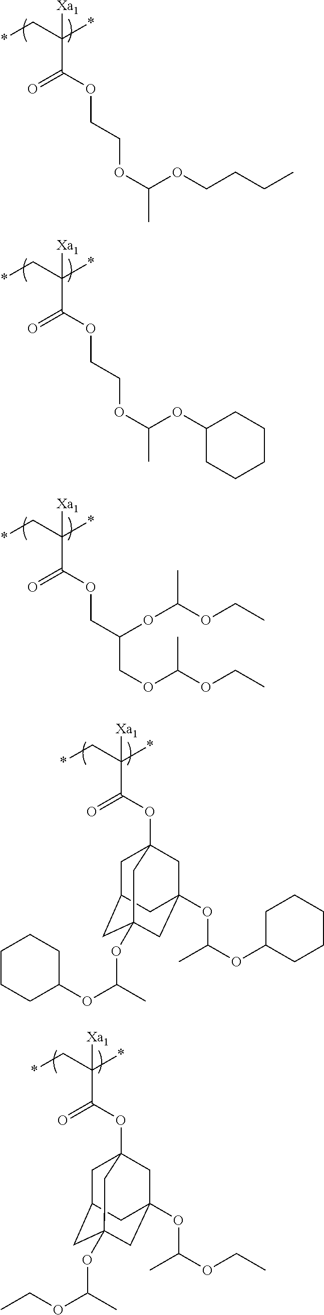

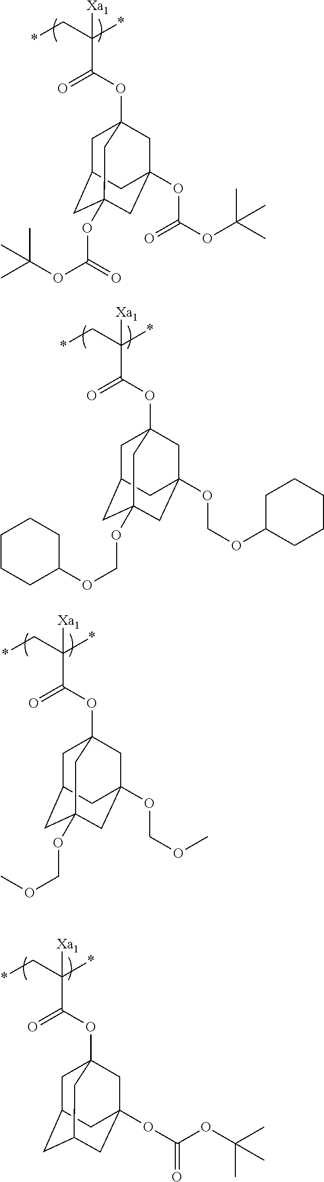

The repeating units which are contained in the resin (B) and which have an acid-decomposable group may be one type, or may be used in a combination of two or more types. In a case of being used in a combination, it is possible to use the specific examples disclosed in paragraph 0287 of US2012/0135348A1; however, the present invention is not limited thereto.

The resin (B) preferably contains a repeating unit which has a cyclic carbonic ester structure in one aspect. The cyclic carbonic ester structure is a structure which has a ring which includes a bond which is represented by —O—C(═O)—O— as the atom group which configures the ring. The ring which includes a bond which is represented by —O—C(═O)—O— as the atom group which configures the ring is preferably a ring with 5 to 7 members, and most preferably a ring with 5 members. Such a ring may be condensed with another ring and form a condensed ring.

The resin (B) preferably contains a repeating unit which has a lactone structure or a sultone (cyclic sulfonic acid ester) structure.

As a lactone group or a sultone group, it is possible to use either as long as the group has a lactone structure or a sultone structure; however, a lactone structure or a sultone structure of a 5 to 7 membered ring is preferable and a lactone group or a sultone group where another ring structure is condensed in a form which forms a bicyclo structure and a spiro structure on a lactone structure or a sultone structure of a 5 to 7 membered ring. It is more preferable to have a repeating unit which has a lactone structure or a sultone structure which is represented by any of General Formula (LC1-1) to (LC1-17) which are disclosed in the paragraph 0318 of US2012/0135348A1 and General Formulae (SL1-1) and (SL1-2) below. In addition, the lactone structure or the sultone structure may be directly bonded with the main chain. Preferable lactone structures or sultone structures are (LC1-1), (LC1-4), (LC1-5), and (LC1-8), and (LC1-4) is more preferable. By using a specific lactone structure or a sultone structure, the LWR and development defects are favorable.

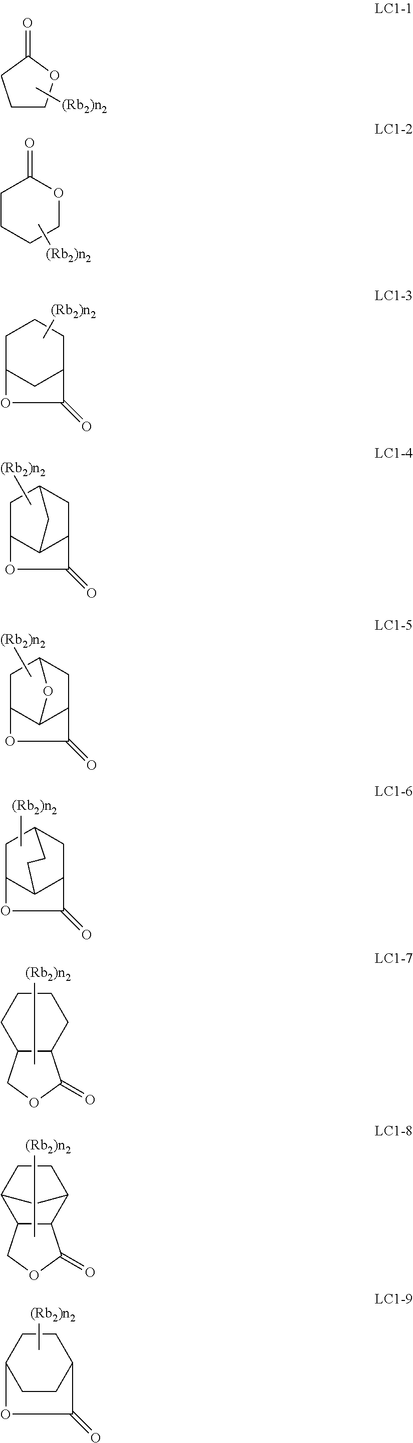

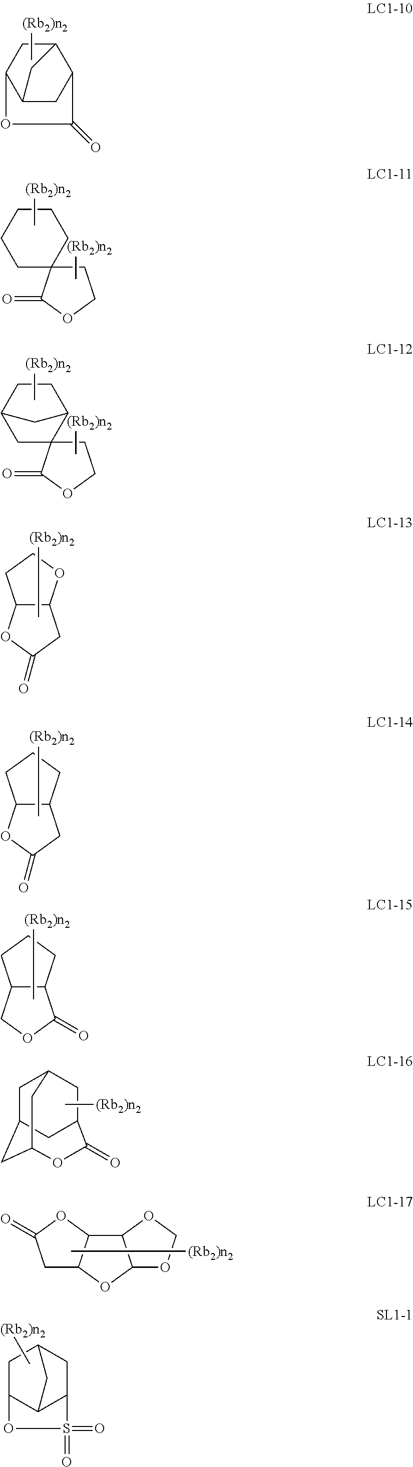

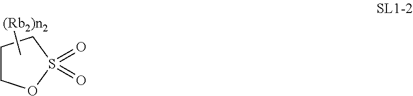

A lactone structure portion or a sultone structure portion may or may not have a substituent group (Rb₂). Examples of the preferable substituent group (Rb₂) include an alkyl group with 1 to 8 carbon atoms, a cycloalkyl group with 4 to 7 carbon atoms, an alkoxy group with 1 to 8 carbon atoms, an alkoxycarbonyl group with 2 to 8 carbon atoms, a carboxyl group, a halogen atom, a hydroxyl group, a cyano group, an acid-decomposable group, and the like. An alkyl group with 1 to 4 carbon atoms, a cyano group, and an acid-decomposable group are more preferable. n₂ represents an integer of 0 to 4. When n₂ is 2 or greater, a plurality of the substituent groups (Rb₂) which are present may be the same or may be different and, additionally, the plurality of substituent groups (Rb₂) which are present may form a ring by bonding with each other.

The resin (B) preferably contains a repeating unit which has a lactone structure or a sultone structure which is represented by General Formula (III) below.

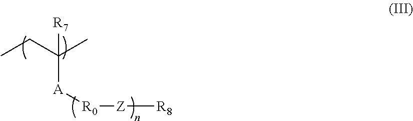

In the Formula (III), A represents an ester bond (a group which is represented by —COO—) or an amide bond (a group which is represented by —CONH—).

In a case where there are a plurality of R₀, each independently represents an alkylene group, a cycloalkylene group, or a combination thereof.

In a case where there are a plurality of Z, each independently represents a single bond, an ether bond, an ester bond, an amide bond, a urethane bond,

(group represented by

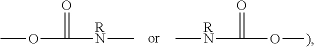

or a urea bond
(group represented by

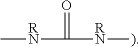

Here, R each independently represents a hydrogen atom, an alkyl group, a cycloalkyl group, or an aryl group.

R₈ represents a monovalent organic group which has a lactone structure and a sultone structure.

n is a repeating number of a structure which is represented by —R₀—Z— and represents an integer of 0 to 2.

R₇ represents a hydrogen atom, a halogen atom, or an alkyl group.

The alkylene group and the cycloalkylene group of R₀ may have a substituent group.

Z is preferably an ether bond and an ester bond, and an ester bond is particularly preferable.

The alkyl group of R₇ is preferably an alkyl group with 1 to 4 carbon atoms, more preferably a methyl group and an ethyl group, and particularly preferably a methyl group. The alkylene group and the cycloalkylene group of R₀ and the alkyl group in R₇ may be each substituted and examples of substituent groups include halogen atoms such as a fluorine atom, a chlorine atom, or a bromine atom, a mercapto group, an alkoxy group such as a hydroxy group, a methoxy group, an ethoxy group, an isopropoxy group, a t-butoxy group, and a benzyloxy group, and an acetoxy group such as an acetyloxy group and a propionyloxy group. R₇ is preferably a hydrogen atom, a methyl group, a trifluoromethyl group, or a hydroxymethyl group.

As a preferable chain alkylene group in R₀, an alkylene group in a chain form with 1 to 10 carbon atoms is preferable, the number of carbon atoms is more preferably 1 to 5, and examples thereof include a methylene group, an ethylene group, a propylene group, and the like. A preferable cycloalkylene group is a cycloalkylene group with 3 to 20 carbon atoms and examples thereof include a cyclohexylene group, a cyclopentylene group, a norbornylene group, an adamantylene group, and the like. In order to exhibit the effects of the present invention, a chained alkylene group is more preferable, and a methylene group is particularly preferable.

A monovalent organic group which has a lactone structure or a sultone structure which is represented by R₈ is not limited as long as the group has a lactone structure or a sultone structure and specific examples thereof include a lactone structure or a sultone structure which is represented by General Formulae (LC1-1) to (LC1-17), and (SL1-1) and (SL1-2) described above and among these, a structure which is represented by (LC1-4) is particularly preferable. In addition, n₂ in (LC1-1) to (LC1-17), and (SL1-1) and (SL1-2) is more preferably 2 or smaller.

In addition, R₈ is preferably a monovalent organic group which has an unsubstituted lactone structure or sultone structure or a monovalent organic group which has a lactone structure or a sultone structure which has a methyl group, a cyano group, or an alkoxycarbonyl group as a substituent group, and a monovalent organic group which has a lactone structure (cyanolactone) or a sultone structure (cyanosultone) which has a cyano group as a substituent group is more preferable.

In General Formula (III), it is preferable that n is 1 or 2.

A is preferably an ester bond.

Z is preferably a single bond.

Specific examples of a repeating unit which has a group which has a lactone structure or a sultone structure which is represented by General Formula (III) include the repeating unit which is disclosed in paragraph 0305 of US2012/0135348A1; however, the present invention is not limited thereto.

As a repeating unit which has a lactone structure or a sultone structure, a repeating unit which is represented by General Formulae (III-1) or (III-1′) below is more preferable.

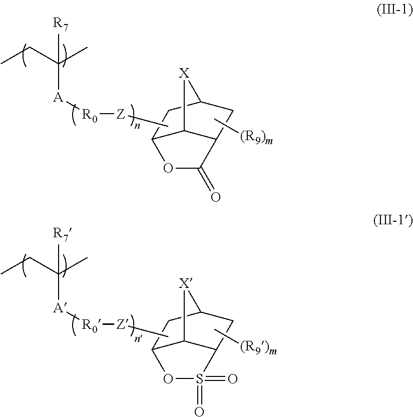

In General Formulae (III-1) and (III-1′), R₇, A, R₀, Z, and n are the same as in General Formula (III) described above.

R₇′, A′, R₀′, Z′, and n′ are the same as R₇, A, R₀, Z, and n in General Formula (III) described above.

In a case where there are a plurality of R₉, each independently represents an alkyl group, a cycloalkyl group, an alkoxycarbonyl group, a cyano group, a hydroxyl group, or an alkoxy group, and in a case where there are a plurality thereof, two R₉ may be bonded with each other to form a ring.

In a case where there are a plurality of R₉′, each independently represents an alkyl group, a cycloalkyl group, an alkoxycarbonyl group, a cyano group, a hydroxyl group, or an alkoxy group, and in a case where there are a plurality thereof, two R₉′ may be bonded with each other to form a ring.

X and X′ represent an alkylene group, an oxygen atom, or a sulfur atom.

m and m′ are the number of substituent groups and represent an integer of 0 to 5. m and m′ are preferably 0 or 1.

As the alkyl group of R₉ and R₉′, an alkyl group with 1 to 4 carbon atoms is preferable, a methyl group and an ethyl group are more preferable, and a methyl group is the most preferable. Examples of the cycloalkyl group include cyclopropyl, cyclobutyl, cyclopentyl, and cyclohexyl groups. Examples of the alkoxycarbonyl group include a methoxycarbonyl group, an ethoxycarbonyl group, an n-butoxycarbonyl group, a t-butoxycarbonyl group, and the like. Examples of the alkoxy group include a methoxy group, an ethoxy group, a propoxy group, an isopropoxy group, a butoxy group, and the like. These groups may have a substituent group and examples of the substituent group include an alkoxy group such as a hydroxy group, a methoxy group, and an ethoxy group; a cyano group; or a halogen atom such as a fluorine atom. More preferably, R₉ and R₉′ are a methyl group, a cyano group, or an alkoxycarbonyl group, and even more preferably a cyano group.

Examples of the alkylene group of X and X′ include a methylene group, an ethylene group, and the like. It is preferably that X and X′ are an oxygen atom or a methylene group, more preferably a methylene group.

In a case where m and m′ are 1 or greater, it is preferable that at least one of R₉ and R₉′ is bonded with the α position or β position of a lactone carbonyl group, particularly preferably bonded with the α position.

Specific examples of a repeating unit which has a group which has a lactone structure, or a sultone structure which is represented by General Formula (III-1) or (III-1′) include the repeating unit which is disclosed in paragraph 0315 of US2012/0135348A1; however, the present invention is not limited thereto.

The content of the repeating unit which is represented by General Formula (III) is preferably 15 mol % to 60 mol %, more preferably 20 mol % to 60 mol %, and even more preferably 30 mol % to 50 mol % with respect to all of the repeating units in the resin (B) by finding the total thereof in a case of containing a plurality of types.

In addition, the resin (B) may contain the repeating unit which has the lactone structure or the sultone structure described above other than a unit which is represented by General Formula (III).

In addition to the specific examples given above, specific examples of a repeating unit which has a lactone group or a sultone group include the repeating units which are disclosed in paragraphs 0325 to 0328 of US2012/0135348A1; however, the present invention is not limited thereto.

Examples of particularly preferable repeating units in the specific examples described above include the repeating unit below. By selecting a suitable lactone group or sultone group, the pattern profile and density dependency become favorable.

### (In the Formula, Rx Represents H, CH₃, CH₂OH, or CF₃)

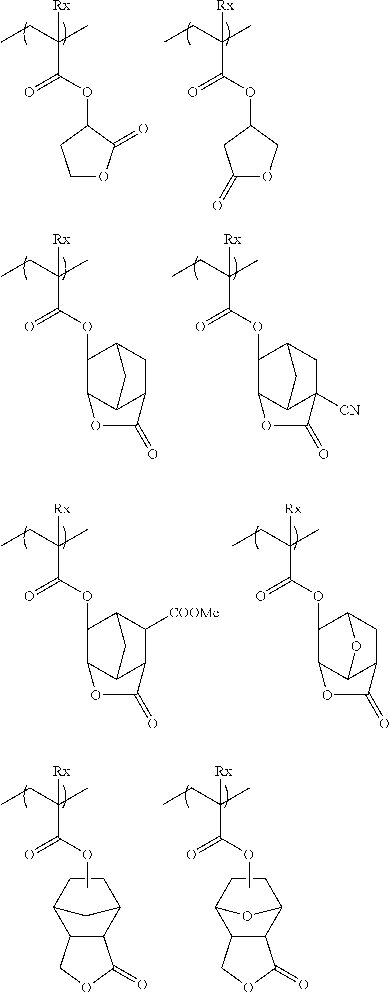

In a repeating unit which has a lactone group or a sultone group, an optical isomer is generally present; however, any optical isomer may be used. In addition, one type of an optical isomer may be used individually or a plurality of optical isomers may be used in a mixture. In a case of mainly using one type of optical isomer, the optical purity (ee) thereof is preferably 90% or more and more preferably 95% or more.

The content of the repeating unit which has a lactone structure or a sultone structure other than a repeating unit which is represented by General Formula (III) is preferably 15 mol % to 60 mol %, more preferably 20 mol % to 50 mol %, and even more preferably 30 mol % to 50 mol % with respect to all of repeating units in the resin by finding the total thereof in a case of containing a plurality of types.

In order to increase the effect of the present invention, it is possible to use two or more types of lactone or sultone repeating units which are selected from General Formula (III) in combination. In a case of using a combination, it is preferable to use a combination by selecting two or more types from lactone or sultone repeating units where n is 1 in General Formula (III).

The resin (B) preferably has a repeating unit which has a hydroxyl group or a cyano group other than in General Formulae (AI) and (III). Due to this, the substrate adhesion and developing solution compatibility are improved. A repeating unit which has a hydroxyl group or a cyano group is preferably a repeating unit which has an alicyclic hydrocarbon structure which is substituted with a hydroxy group or a cyano group and it is preferable not to have an acid-decomposable group. As an alicyclic hydrocarbon structure in an alicyclic hydrocarbon structure which is substituted with a hydroxyl group or a cyano group, an adamantyl group, diadamantyl group, and a norbornane group are preferable. As a preferable alicyclic hydrocarbon structure which is substituted with a hydroxyl group or a cyano group, a partial structure which is represented by General Formulae (VIIa) to (VIId) below is preferable.

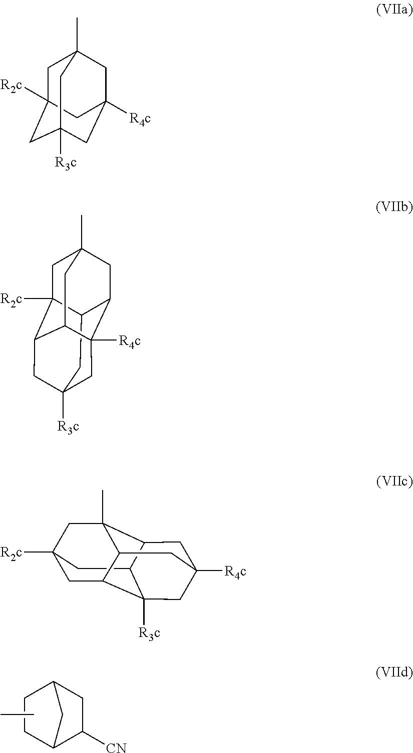

In General Formulae (VIIa) to (VIIc), R₂c to R₄c each independently represents a hydrogen atom, a hydroxyl group, or a cyano group. However, at least one out of R₂c to R₄c represents a hydroxyl group or a cyano group. Preferably, one or two out of R₂c to R₄c are a hydroxyl group and the rest are a hydrogen atom. In General Formula (VIIa), more preferably, two out of R₂c to R₄c are a hydroxy group and the rest are a hydrogen atom.

Examples of a repeating unit which has a partial structure which is represented by General Formulae (VIIa) to (VIId) include a repeating unit which is represented by General Formulae (AIIa) to (AIId) below.

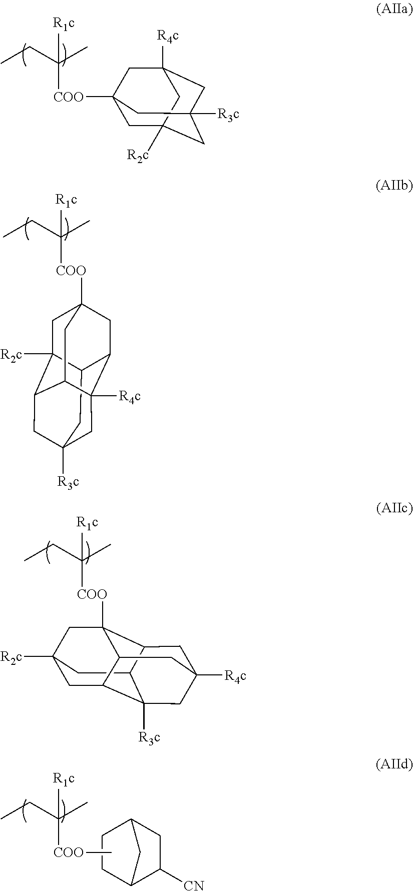

In General Formulae (AIIa) to (AIId), R₁c represents a hydrogen atom, a methyl group, a trifluoromethyl group, or a hydroxymethyl group.

R₂c to R₄c are the same as R₂c to R₄c in General Formulae (VIIa) to (VIIc).

The content of a repeating unit which has a hydroxyl group or a cyano group is preferably 5 mol % to 40 mol %, more preferably 5 mol % to 30 mol %, and even more preferably 10 mol % to 25 mol % with respect to all of the repeating units in the resin (B).

Specific examples of a repeating unit which has a hydroxyl group or a cyano group include the repeating unit which is disclosed in paragraph 0340 of US2012/0135348A1; however, the present invention is not limited thereto.

The resin (B) which is used for the actinic-ray-sensitive or radiation-sensitive resin composition of the present invention may have a repeating unit which has an alkali-soluble group. Examples of an alkali-soluble group include a carboxyl group, a sulfonamide group, a sulfonylimide group, a bissulfonylimide group, and aliphatic alcohol where the α position is substituted with an electron-withdrawing group (for example, a hexafluoroisopropanol group), and it is more preferable to have a repeating unit which has a carboxyl group. By containing a repeating unit which has an alkali-soluble group, the resolution for contact hole usage increases. As a repeating unit which has an alkali-soluble group, either of a repeating unit where an alkali-soluble group is directly bonded with a main chain of a resin such as a repeating unit by acrylic acid and methacrylic acid or a repeating unit where an alkali-soluble group is bonded with a main chain of a resin via a bonding group is preferable, and the bonding group may have a monocyclic or polycyclic cyclic hydrocarbon structure. A repeating unit using acrylic acid and methacrylic acid is particularly preferable. In addition, the resin (B) may be a resin where an alkali-soluble group is bonded with an end of a polymer chain which is used and prepared when polymerizing a polymerization initiator or a chain transfer agent which has an alkali-soluble group.

The content of a repeating unit which has an alkali-soluble group is preferably 0 mol % to 20 mol %, more preferably 3 mol % to 15 mol %, and even more preferably 5 mol % to 10 mol % with respect to all of the repeating units in the resin (B).

Specific examples of a repeating unit which has an alkali-soluble group include the repeating units which are disclosed in paragraph 0344 of US2012/0135348A1; however, the present invention is not limited thereto.

It is possible for the resin (B) of the present invention to further have a repeating unit which has an alicyclic hydrocarbon structure which does not have a polar group (for example, the alkali-soluble group, hydroxy group, cyano group, or the like) and which does not exhibit acid decomposability. Examples of such a repeating unit include the repeating unit which is represented by General Formula (IV).

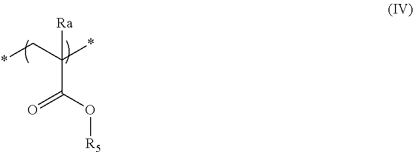

In General Formula (IV) described above, R₅ represents a hydrocarbon group which has at least one ring structure and does not have a polar group.

Ra represents a hydrogen atom, an alkyl group, or a —CH₂—O—Ra₂ group. In the formula, Ra₂ represents a hydrogen atom, an alkyl group, or an acyl group. Ra is preferably a hydrogen atom, a methyl group, a hydroxymethyl group, or a trifluoromethyl group, and particularly preferably a hydrogen atom or a methyl group.

The ring structure of R₅ includes a monocyclic hydrocarbon group and a polycyclic hydrocarbon group. Examples of monocyclic hydrocarbon groups include a cycloalkyl group with 3 to 12 carbon atoms such as a cyclopentyl group, a cyclohexyl group, a cycloheptyl group, or a cyclooctyl group, and a cycloalkenyl group with 3 to 12 carbon atoms such as a cyclohexenyl group. A preferable monocyclic hydrocarbon group is a monocyclic hydrocarbon group with 3 to 7 carbon atoms, and more preferable examples thereof include a cyclopentyl group and a cyclohexyl group.

The polycyclic hydrocarbon group includes a ring-aggregated hydrocarbon group and a crosslinked cyclic hydrocarbon group and examples of the ring-aggregated hydrocarbon group include a bicyclohexyl group, a perhydronaphthalenyl group, and the like. Examples of crosslinked cyclic hydrocarbon rings include a 2-ring type hydrocarbon ring such as pinane, bornane, norpinane, norbornane, or a bicyclooctane ring (a bicyclo[2.2.2]octane ring, a bicyclo[3.2.1]octane ring, and the like), a 3-ring type hydrocarbon ring such as homobredene, adamantane, tricyclo[5.2.1.0^{2,6}]decane, or a tricyclo[4.3.1.1^{2.5}]undecane ring, and a 4-ring type hydrocarbon ring such as tetracyclo[4.4.0.12,5.1^{7,10}]dodecane or a perhydro-1,4-methano-5,8-methanonaphthalene ring. In addition, the crosslinked cyclic hydrocarbon group also includes a condensed cyclic hydrocarbon ring, for example, a condensed ring where a plurality of 5 to 8 membered cycloalkane rings are condensed such as perhydronaphthalene (decaline), perhydroanthracene, perhydrophenanthrene, perhydroacenaphthene, perhydrofluorene, perhydroindene, and perhydrophenalene rings.

Examples of a preferable crosslinked cyclic hydrocarbon group include a norbornyl group, an adamantyl group, a bicyclooctanyl group, a tricyclo[5,2,1,0^{2,6}]decanyl group, and the like. Examples of a more preferable crosslinked cyclic hydrocarbon group include a norbornyl group and an adamantyl group.

The alicyclic hydrocarbon group may have a substituent group and examples of a preferable substituent group include a halogen atom, an alkyl group, a hydroxyl group where a hydrogen atom is substituted, an amino group where a hydrogen atom is substituted, and the like. Examples of preferable halogen atoms include bromine, chlorine, fluorine atoms and examples of preferable alkyl groups include methyl, ethyl, butyl, and t-butyl groups. The alkyl group described above may further have a substituent group and examples of a substituted group which the alkyl group may further have include a halogen atom, an alkyl group, a hydroxyl group where a hydrogen atom is substituted and an amino group where a hydrogen atom is substituted.

Examples of the group where the hydrogen atom described above is substituted include an alkyl group, a cycloalkyl group, an aralkyl group, a substituted methyl group, a substituted ethyl group, an alkoxycarbonyl group, and an aralkyloxycarbonyl group. Examples of a preferable alkyl group include an alkyl group with 1 to 4 carbon atoms, examples of a preferable substituted methyl group include methoxymethyl, methoxythiomethyl, benzyloxymethyl, t-butoxymethyl, and 2-methoxyethoxymethyl groups, examples of a substituted ethyl group include 1-ethoxyethyl and 1-methyl-1-methoxyethyl, examples of a preferable acyl group include aliphatic acyl groups with 1 to 6 carbon atoms such as formyl, acetyl, propionyl, butyryl, isobutyryl, valeryl, and pivaloyl groups, and examples of an alkoxycarbonyl group include an alkoxycarbonyl group with 1 to 4 carbon atoms and the like.

The resin (B) may or may not contain a repeating unit which has an alicyclic hydrocarbon structure which does not have a polar group and does not exhibit acid decomposability; however, when contained, the content of the repeating unit is preferably 1 mol % to 40 mol % and more preferably 2 mol % to 20 mol % with respect to all of the repeating units in the resin (B).

Specific examples of a repeating unit which has an alicyclic hydrocarbon structure which does not have a polar group and does not exhibit acid decomposability include the repeating units which are disclosed in paragraph 0354 of US2012/0135348A1; however, the present invention is not limited thereto.

The resin (B) which is used for the composition of the present invention is able to have various types of repeating units other than the repeating units described above for the purpose of adjusting the dry etching resistance or standard developing solution aptitude, the substrate adhesion, the resist profile, in addition to the resolving power, heat resistance, sensitivity, and the like which are typical necessary characteristics for the resists.

Examples of the repeating unit include repeating units which are equivalent to the monomer below; however, the present invention is not limited thereto.

Due to this, it is possible to carry out fine adjustment of the properties which are demanded for the resins which are used for the compositions of the present invention, in particular, (1) solubility with respect to a coating solvent, (2) film-forming property (glass transition point), (3) alkali developing characteristics, (4) film thinning (selecting hydrophilic-hydrophobic properties and alkali-soluble groups), (5) adhesion of a non-exposed section to a substrate, (6) dry etching resistance, and the like.

Examples of such monomers include compounds or the like which have one addition polymerizable unsaturated bond which is selected from, for example, acrylic acid esters, methacrylic acid esters, acrylamides, methacrylamides, allyl compounds, vinylethers, vinylesters, and the like.

Apart from the above, copolymerizing may be carried out in the case of an addition polymerizable unsaturated compound which is able to be copolymerized with a monomer which is equivalent to the various types of repeating units described above.

In the resin (B) which is used for the composition of the present invention, the content molar ratio of each repeating unit is appropriately set in order to adjust the dry etching resistance or standard developing solution aptitude, the substrate adhesion, and the resist profile of the resist, in addition to the resolving power, heat resistance, sensitivity, and the like which are typical necessary characteristics for resists.

When the composition of the present invention is used for ArF exposure, it is preferable that the resin (B) which is used for the composition of the present invention substantially does not have an aromatic group from the point of view of transparency to ArF light. In more detail, in all of the repeating units of the resin (B), it is preferable that repeating units which have an aromatic group are 5 mol % or less of the whole, more preferably 3 mol % or less, ideally 0 mol %, that is, it is even more preferable not to have a repeating unit which has an aromatic group. In addition, it is preferable that the resin (B) has a monocyclic or polycyclic alicyclic hydrocarbon structure.

Here, from the point of view of solubility with a hydrophobic resin (HR) which will be described below, it is preferable that the resin (B) does not contain a fluorine atom and a silicon atom.

The resin (B) which is used for the composition of the present invention is preferably a resin where all of the repeating units are configured by (meth)acrylate-based repeating units. In this case, it is possible to use any of a resin where all of the repeating units are methacrylate-based repeating units, a resin where all of the repeating units are acrylate-based repeating units, and a resin where all of the repeating units are methacrylate-based repeating units and acrylate-base repeating units; however, it is preferable that the acrylate-based repeating units are 50 mol % or less of all of the repeating units. In addition, a copolymer which includes 20 mol % to 50 mol % of (meth)acrylate-based repeating units which have an acid-decomposable group, 20 mol % to 50 mol % of (meth)acrylate-based repeating units which have a lactone group, 5 mol % to 30 mol % of (meth)acrylate-based repeating units which have an alicyclic hydrocarbon structure which is substituted with a hydroxyl group or a cyano group, in addition to 0 mol % to 20 mol % of other (meth)acrylate-based repeating units is also preferable.

In a case of irradiating the composition of the present invention with KrF excimer laser light, electron beams, X-rays or high energy rays with wavelength of 50 nm or less (for example, EUV), it is preferable that the resin (B) has a hydroxystyrene repeating unit. Even more preferably, the resin (B) is a copolymer of hydroxystyrene and hydroxystyrene which is protected by a group which leaves by an action of an acid, or a copolymer of hydroxystyrene and (meth)acrylic acid tertiary alkylester.

Specific examples of the resin include a resin which has a repeating unit which is represented by General Formula (A) below.

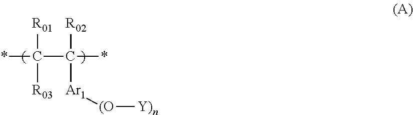

In the formula, R₀₁, R₀₂ and R₀₃ each independently represents a hydrogen atom, an alkyl group, a cycloalkyl group, a halogen atom, a cyano group, or an alkoxycarbonyl group. Ar₁ represents, for example, an aromatic ring group. Here, R₀₃ and Ar₁ are alkylene groups and may form a 5 membered or 6 membered ring with the —C—C-chain by being bonded with each other.

A number n of Y each independently represents a hydrogen atom or a group which leaves by an action of an acid. However, at least one Y represents a group which leaves by an action of an acid.

n represents an integer of 1 to 4, preferably 1 or 2, and more preferably 1.

An alkyl group as R₀₁ to R₀₃ is, for example, an alkyl group with 20 or fewer carbon atoms and preferably a methyl group, an ethyl group, a propyl group, an isopropyl group, an n-butyl group, a sec-butyl group, a hexyl group, a 2-ethylhexyl group, an octyl group, or a dodecyl group. More preferably, the alkyl groups are an alkyl group with 8 or fewer carbon atoms. Here, the alkyl groups may have a substituent group.

As an alkyl group which is included in an alkoxycarbonyl group, the same alkyl group as the alkyl group in R₀₁ to R₀₃ described above is preferable.

The cycloalkyl group may be a monocyclic cycloalkyl group or a polycyclic cycloalkyl group. Preferable examples thereof include a monocyclic cycloalkyl group with 3 to 8 carbon atoms such as a cyclopropyl group, cyclopentyl group, and a cyclohexyl group. Here, the cycloalkyl group may have a substituent group.

Examples of halogen atoms include a fluorine atom, a chlorine atom, a bromine atom, and an iodine atom, and a fluorine atom is more preferable.

In a case where R₀₃ represents an alkylene group, preferable examples of the alkylene group include an alkylene group with 1 to 8 carbon atoms such as a methylene group, an ethylene group, a propylene group, a butylene group, a hexylene group, and an octylene group.

An aromatic ring group as Ar₁ is preferably an aromatic ring group with 6 to 14 carbon atoms, and examples thereof include a benzene ring, a toluene ring, and a naphthalene ring. Here, the aromatic ring groups may have a substituent group.

Examples of a group Y which leaves by an action of an acid include a groups which are represented by —C(R₃₆)(R₃₇)(R₃₈), —C(═O)—O—C(R₃₆)(R₃₇)(R₃₈), —C(R₀₁)(R₀₂)(OR₃₉), —C(R₀₁)(R₀₂)—C(═O)—O—C(R₃₆)(R₃₇)(R₃₈), and —CH(R₃₆)(Ar).

In the formula, R₃₆ to R₃₉ each independently represents an alkyl group, a cycloalkyl group, an aryl group, an aralkyl group, or an alkenyl group. R₃₆ and R₃₇ may form a ring structure by bonding with each other.

R₀₁ and R₀₂ each independently represents a hydrogen atom, an alkyl group, a cycloalkyl group, an aryl group, an aralkyl group, or an alkenyl group.

Ar represents an aryl group.

An alkyl group as R₃₆ to R₃₉, R₀₁, or R₀₂ is preferably an alkyl group with 1 to 8 carbon atoms and examples thereof include a methyl group, an ethyl group, a propyl group, an n-butyl group, a sec-butyl group, a hexyl group, and an octyl group.

A cycloalkyl group as R₃₆ to R₃₉, R₀₁, or R₀₂ may be a monocyclic cycloalkyl group or may be a polycyclic cycloalkyl group. A monocyclic cycloalkyl group is preferably a cycloalkyl group with 3 to 8 carbon atoms and examples include a cyclopropyl group, a cyclobutyl group, a cyclopentyl group, a cyclohexyl group, and a cyclooctyl group. A polycyclic cycloalkyl group is preferably a cycloalkyl group with 6 to 20 carbon atoms and examples thereof include an adamantyl group, a norbornyl group, an isoboronyl group, a camphanyl group, a dicyclopentyl group, an α-pinanyl group, a tricyclodecanyl group, a tetracyclododecyl group, and an androstanyl group. Here, some of the carbon atoms in the cycloalkyl group may be substituted with hetero atoms such as an oxygen atom.

An aryl group as R₃₆ to R₃₉, R₀₁, R₀₂, or Ar is preferably an aryl group with 6 to 10 carbon atoms and examples thereof include a phenyl group, a naphthyl group, and an anthryl group.

An aralkyl group as R₃₆ to R₃₉, R₀₁, or R₀₂ is preferably an aralkyl group with 7 to 12 carbon atoms and, for example, a benzyl group, a phenethyl group, and a naphthylmethyl group are preferable.

An alkenyl group as R₃₆ to R₃₉, R₀₁, or R₀₂ is preferably an alkenyl group with 2 to 8 carbon atoms and examples thereof include a vinyl group, an allyl group, a butenyl group, and a cyclohexenyl group.

A ring formed by R₃₆ and R₃₇ bonding with each other may be monocyclic or may be polycyclic. As a monocyclic type, a cycloalkane structure with 3 to 8 carbon atoms is preferable and examples thereof include a cyclopropane structure, a cyclobutane structure, a cyclopentane structure, a cyclohexane structure, a cycloheptane structure, and a cyclooctane structure. As a polycyclic type, a cycloalkane structure with 6 to 20 carbon atoms is preferable and examples thereof include an adamantane structure, a norbornane structure, a dicyclopentane structure, a tricyclodecane structure, and a tetracyclododecane structure. Here, some of the carbon atoms in the ring structure may be substituted with hetero atoms such as an oxygen atom.

Each of the groups described above may have a substituent group. Examples of the substituent group include an alkyl group, a cycloalkyl group, an aryl group, an amino group, an amide group, a ureide group, a urethane group, a hydroxyl group, a carboxyl group, a halogen atom, an alkoxy group, a thioether group, an acyl group, an acyloxy group, an alkoxycarbonyl group, a cyano group, and a nitro group. The substituent groups preferably have 8 or fewer carbon atoms.

As a group Y which leaves by an action of an acid, a structure which is represented by General Formula (B) below is more preferable.

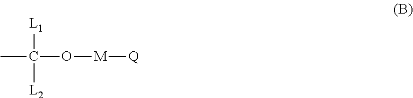

In the formula, L₁ and L₂ each independently represents a hydrogen atom, an alkyl group, a cycloalkyl group, an aryl group, or an aralkyl group.

M represents a single bond or a divalent bonding group.

Q represents an alkyl group, a cycloalkyl group, a cyclic aliphatic group, an aromatic ring group, an amino group, an ammonium group, a mercapto group, a cyano group, or an aldehyde group. Here, the cyclic aliphatic group and an aromatic ring group may include a hetero atom.

Here, a 5 membered or 6 membered ring may be formed by at least two of Q, M, and L₁ being bonded with each other.

An alkyl group as L₁ and L₂ is, for example, an alkyl group with 1 to 8 carbon atoms, and specific examples thereof include a methyl group, an ethyl group, a propyl group, an n-butyl group, a sec-butyl group, a hexyl group, and an octyl group.

A cycloalkyl group as L₁ and L₂ is, for example, a cycloalkyl group with 3 to 15 carbon atoms, and specific examples include a cyclopentyl group, a cyclohexyl group, a norbornyl group, and an adamantyl group.

An aryl group as L₁ and L₂ is, for example, an aryl group with 6 to 15 carbon atoms and specific examples thereof include a phenyl group, a tolyl group, a naphthyl group, and an anthryl group.

An aralkyl group as L₁ and L₂ is, for example, an aralkyl group with 6 to 20 carbon atoms, and specific examples thereof include a benzyl group and a phenethyl group.

A divalent bonding group as M is, for example, an alkylene group (for example, a methylene group, an ethylene group, a propylene group, a butylene group, a hexylene group, or an octylene group), a cycloalkylene group (for example, a cyclopentylene group or a cyclohexylene group), an alkenylene group (for example, an ethylene group, a propenylene group, or a butenylene group), an arylene group (for example, a phenylene group, a tolylene group, or a naphthylene group), —S—, —O—, —CO—, —SO₂—, —N(R₀)—, or a combination of two or more thereof. Here, R₀ is a hydrogen atom or an alkyl group. An alkyl group as R₀ is, for example, an alkyl group with 1 to 8 carbon atoms, and specific examples thereof include a methyl group, an ethyl group, a propyl group, an n-butyl group, a sec-butyl group, a hexyl group, and an octyl group.

An alkyl group and a cycloalkyl group as Q are the same as each of the groups as L₁ and L₂ described above.

Examples of a cyclic aliphatic group or an aromatic ring group as Q include the cycloalkyl group and the aryl group as L₁ and L₂ described above. The cycloalkyl group and the aryl group are preferably groups with 3 to 15 carbon atoms.

Examples of a cyclic aliphatic group or an aromatic ring group which include hetero atoms as Q include groups such as thiirane, cyclothiolane, thiophene, furan, pyrrole, benzothiophene, benzofuran, benzopyrrole, triazine, imidazole, benzoimidazole, triazole, thiadiazole, thiazole, pyrrolidone, and the like which have a heterocyclic structure. However, the present invention is not limited thereto as long as the ring is a ring which is formed by carbon and hetero atoms or a ring which is formed by only hetero atoms.

Examples of a ring structure which at least two of Q, M, and L₁ may form by bonding with each other include a 5 membered or 6 membered ring structure which is formed by these forming a propylene group or a butylene group. Here, the 5 membered or 6 membered ring structure contain oxygen atoms.

Each of the groups which are represented by L₁, L₂, M, and Q in General Formula (B) may have a substituent group. Examples of the substituent group include an alkyl group, a cycloalkyl group, an aryl group, an amino group, an amide group, a ureide group, a urethane group, a hydroxyl group, a carboxyl group, a halogen atom, an alkoxy group, a thioether group, an acyl group, an acyloxy group, an alkoxycarbonyl group, a cyano group, and a nitro group. The substituent groups preferably have 8 or fewer carbon atoms.

As a group which is represented by -(M-Q), a group with 1 to 20 carbon atoms is preferable, a group with 1 to 10 carbon atoms is more preferable, and a group with 1 to 8 carbon atoms is even more preferable.

Specific examples of a resin (A1) which has a hydroxystyrene repeating unit will be given below; however, the present invention is not limited thereto.

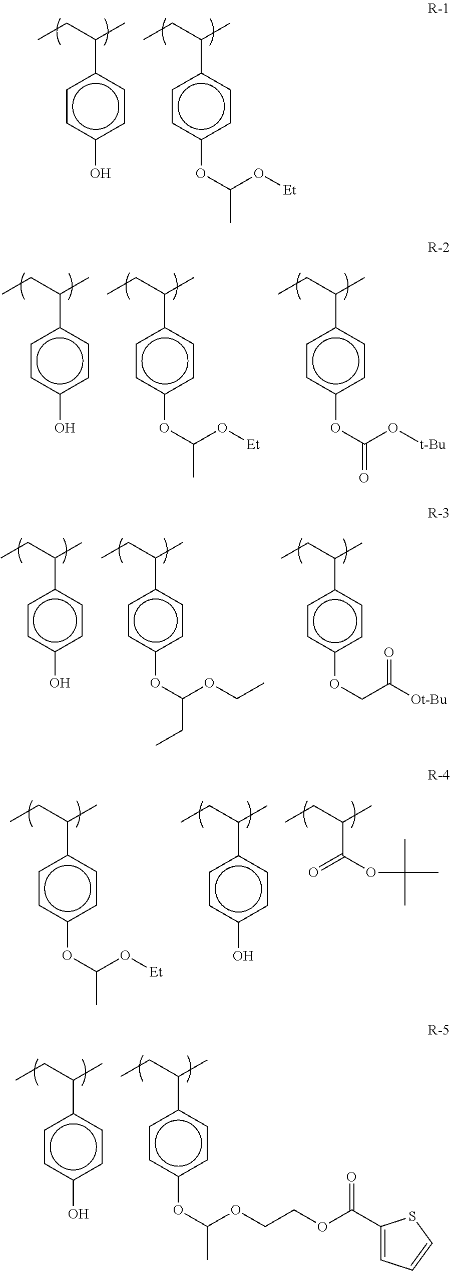

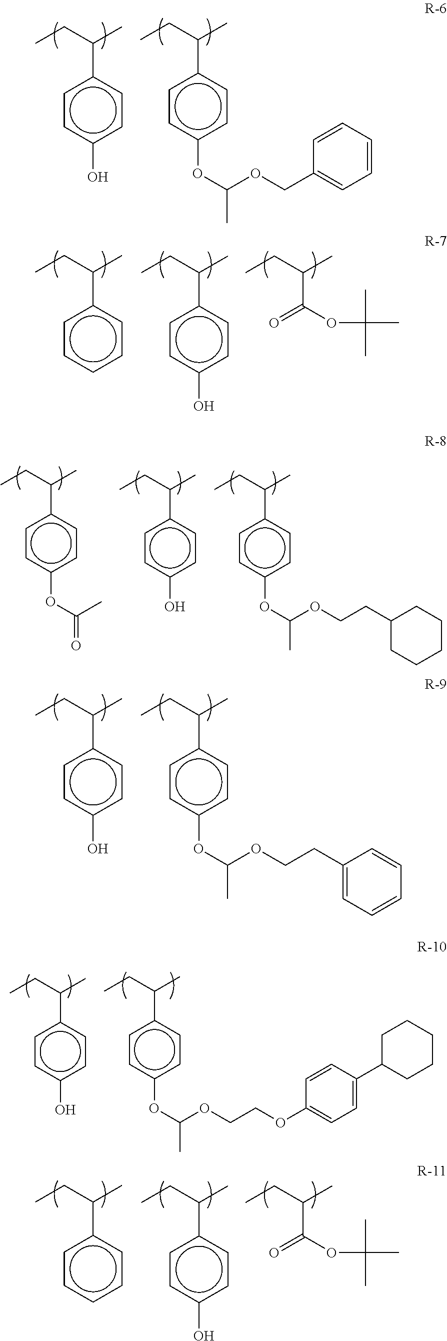

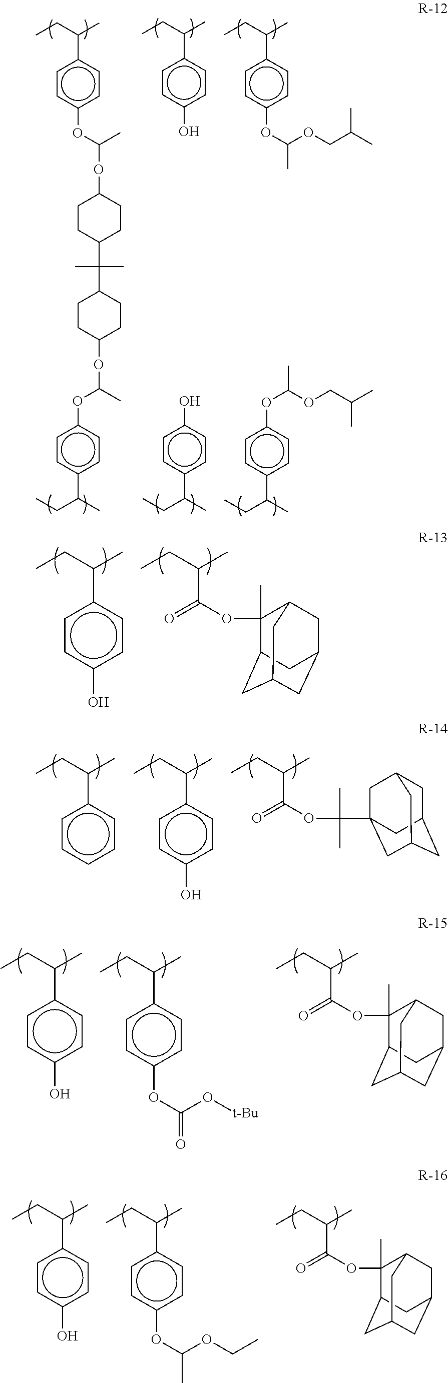

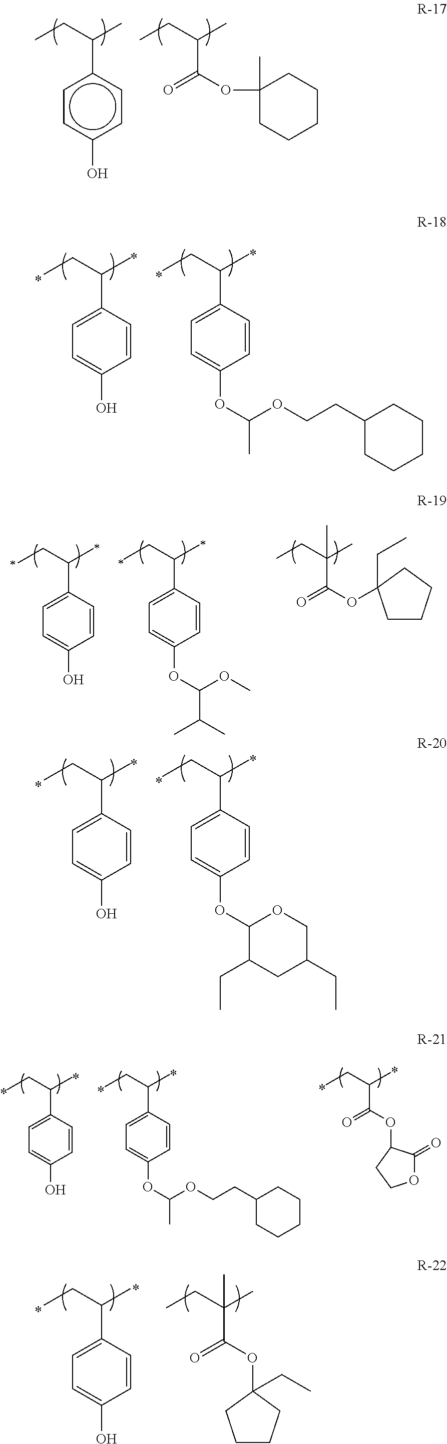

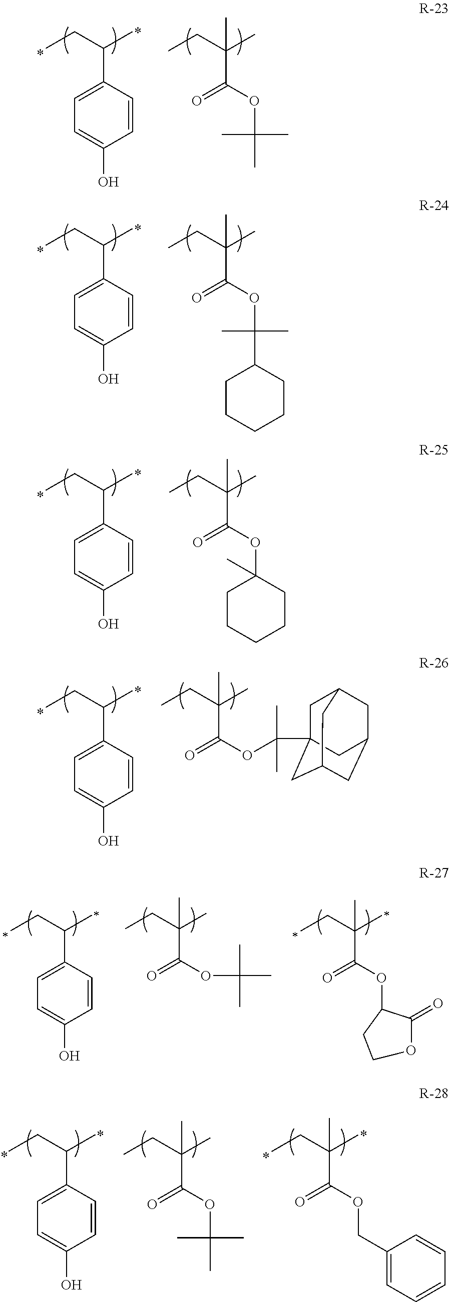

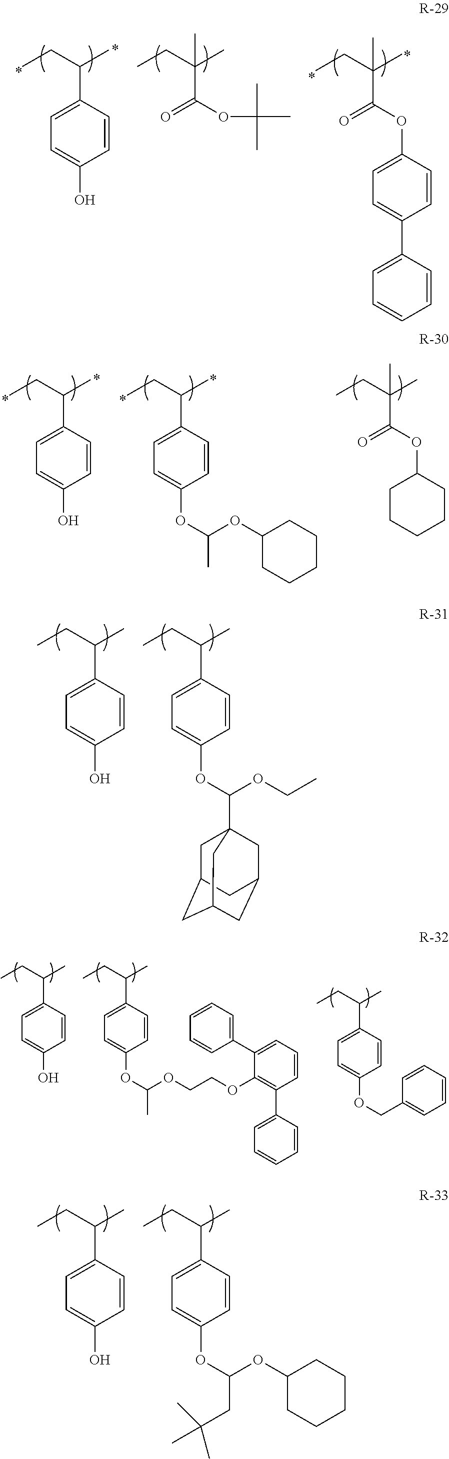

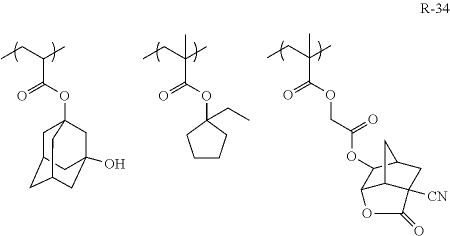

In the specific examples described above, t-Bu represents a t-butyl group.

It is possible to synthesize the resin (B) in the present invention by a routine procedure (for example, radical polymerization). In detail, it is possible to use a synthesis method which is disclosed in paragraphs 0126 to 0128 of US2012/0164573A.

A weight average molecular weight of the resin (B) of the present invention is preferably 1,000 to 200,000, more preferably 2,000 to 20,000, even more preferably 3,000 to 15,000, and particularly preferably 3,000 to 11,000 as a polystyrene converted value by a GPC method. By setting the weight average molecular weight to 1,000 to 200,000, it is possible to prevent a deterioration in the heat resistance or dry etching resistance and it is possible to prevent the developing characteristics from deteriorating or the film-forming property from deteriorating due to the viscosity being increased.

The dispersity (molecular weight distribution: Mw/Mn) is generally 1.0 to 3.0 and ranges of preferably 1.0 to 2.6, more preferably 1.0 to 2.0, and particularly preferably 1.4 to 2.0 are used. When the molecular weight distribution is small, the resolution and a resist shape are excellent and a side wall of a resist pattern is smooth and the roughness is excellent.

It is possible to measure the weight average molecular weight (Mw) and the number average molecular weight (Mn) in the present specification by gel permeation chromatography (GPC).

The GPC uses an HLC-8020 (manufactured by Tosoh corporation), where a TSK gel Multipore HXL-M (manufactured by Tosoh corporation, 7.8 mmID×30.0 cm) is used as a column, and tetrahydrofuran (THF) is used as an eluent.

The content ratio in the entire composition of the resin (B) in the present invention is preferably 30 mass % to 99 mass % in the total solid content and more preferably 55 mass % to 95 mass %.

In addition, the resin (B) in the present invention may be used as one type or a plurality thereof may be used in combination.

[3] Basic Compound

The actinic-ray-sensitive or radiation-sensitive resin composition of the present invention preferably contains a basic compound in order to reduce changes in the performance due to the passing of time from exposure to heating.

Examples of preferable basic compounds include a compound which has a structure which is shown by Formulae (A) to (E) below.

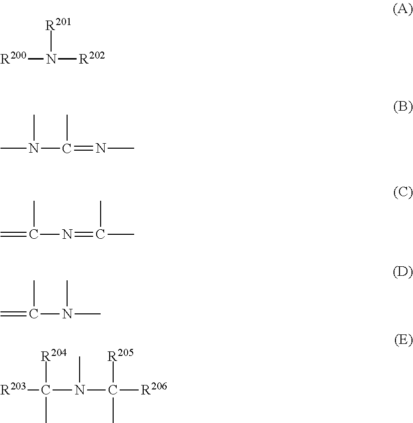

In General Formulae (A) and (E), R²⁰⁰, R²⁰¹, R²⁰² may be the same or may be different and represent a hydrogen atom, an alkyl group (preferably with 1 to 20 carbon atoms), a cycloalkyl group (preferably with 3 to 20 carbon atoms), and an aryl group (with 6 to 20 carbon atoms) and here, R²⁰¹ and R²⁰² may form a ring by bonding with each other.

R²⁰³, R²⁰⁴, R²⁰⁵, and R²⁰⁶ may be the same or may be different and represent an alkyl group with 1 to 20 carbon atoms.

With regard to the alkyl group described above, as an alkyl group which has a substituent group, an aminoalkyl group with 1 to 20 carbon atoms, a hydroxyalkyl group with 1 to 20 carbon atoms, or a cyanoalkyl group with 1 to 20 carbon atoms is preferable.

More preferably, the alkyl groups in General Formulae (A) and (E) are unsubstituted.

Examples of a preferable compound include guanidine, aminopyrrolidine, pyrazole, pyrazoline, piperazine, aminomorpholine, aminoalkyl morpholine, piperidine, and the like, and examples of more preferable compounds include a compound which has an imidazole structure, a diazabicyclo structure, an onium hydroxide structure, an onium carboxylate structure, a trialkylamine structure, an aniline structure, or a pyridine structure, an alkylamine derivative which has a hydroxyl group and/or an ether bond, an aniline derivative which has a hydroxyl group and/or an ether bond, and the like.

Examples of a compound which has an imidazole structure include imidazole, 2,4,5-triphenylimidazole, benzimidazole, 2-phenylbenzoimidazole, and the like. Examples of a compound which has a diazabicyclo structure include 1,4-diazabicyclo[2,2,2]octane, 1,5-diazabicyclo[4,3,0]nona-5-en, 1,8-diazabicyclo[5,4,0]undeca-7-en, and the like. Examples of a compound which has an onium hydroxide structure include tetrabutylammonium hydroxide, triarylsulfonium hydroxide, phenacylsulfonium hydroxide, sulfonium hydroxide which has a 2-oxoalkyl group, specifically, triphenylsulfonium hydroxide, tris(t-butylphenyl) sulfonium hydroxide, bis(t-butylphenyl) iodonium hydroxide, phenacyl thiophenium hydroxide, 2-oxopropylthiophenium hydroxide, and the like. With regard to a compound which has an onium carboxylate structure, an anion section of a compound which has an onium hydroxide structure is a carboxylate and examples thereof include acetate, adamantane-1-carboxylate, perfluoroalkyl carboxylate, and the like. Examples of a compound which has a trialkylamine structure include tri(n-butyl)amine, tri(n-octyl)amine, and the like. Examples of a compound which has an aniline structure include 2,6-diisopropylaniline, N,N-dimethylaniline, N,N-dibutylaniline, N,N-dihexylaniline, and the like. Examples of an alkylamine derivative which has a hydroxyl group and/or an ether bond include ethanolamine, diethanolamine, triethanolamine, N-phenyldiethanolamine, tris(methoxyethoxyethyl)amine, and the like. Examples of an aniline derivative which has a hydroxyl group and/or an ether bond include N,N-bis(hydroxyethyl) aniline, and the like.

Examples of preferable basic compounds further include an amine compound which has a phenoxy group, an ammonium salt compound which has a phenoxy group, an amine compound which has a sulfonic acid ester group, and an ammonium salt compound which has a sulfonic acid ester group.

It is possible to use a primary, secondary, and tertiary amine compound as the amine compound, and an amine compound where at least one alkyl group is bonded with a nitrogen atom is preferable. More preferably, the amine compound is a tertiary amine compound. With regard to the amine compound, when at least one alkyl group (preferably with 1 to 20 carbon atoms) is bonded with a nitrogen atom, in addition to the alkyl group, a cycloalkyl group (preferably with 3 to 20 carbon atoms) or an aryl group (preferably with 6 to 12 carbon atoms) may be bonded with the nitrogen atom. The amine compound preferably has oxygen atoms in an alkyl chain and that an oxyalkylene group is formed. The number of the oxyalkylene groups in the molecule is 1 or more, preferably 3 to 9, and more preferably 4 to 6. Among oxyalkylene groups, an oxyethylene group (—CH₂CH₂O—) or an oxypropylene group (—CH(CH₃)CH₂O— or —CH₂CH₂CH₂O—) is preferable and an oxyethylene group is more preferable.

It is possible to use a primary, secondary, tertiary, or quaternary ammonium salt compound for an ammonium salt compound, and an ammonium salt compound where at least one alkyl group is bonded with a nitrogen atom is preferable. With regard to the ammonium salt compound, when at least one alkyl group (preferably with 1 to 20 carbon atoms) is bonded with a nitrogen atom, in addition to the alkyl group, a cycloalkyl group (preferably with 3 to 20 carbon atoms) or an aryl group (preferably with 6 to 12 carbon atoms) may be bonded with the nitrogen atom. The ammonium salt compound preferably has oxygen atoms in the alkyl chain and that an oxyalkylene group is formed. It is preferable to have one or more oxyalkylene groups in the molecule, it is more preferable to have 3 to 9 in the molecule, and it is even more preferable to have 4 to 6 in the molecule. Among oxyalkylene groups, an oxyethylene group (—CH₂CH₂O—) or an oxypropylene group (—CH(CH₃)CH₂O— or —CH₂CH₂CH₂O—) is preferable and an oxyethylene group is more preferable.

Examples of an anion of an ammonium salt compound include a halogen atom, sulfonate, borate, phosphate, and the like; however, among these, a halogen atom and sulfonate are preferable. As a halogen atom, chloride, bromide, and iodide are particularly preferable and as sulfonate, organic sulfonate with 1 to 20 carbon atoms is particularly preferable. Examples of organic sulfonate include alkylsulfonate and arylsulfonate with 1 to 20 carbon atoms. An alkyl group of alkylsulfonate may have a substituent group, and examples of the substituent group include fluorine, chlorine, bromine, an alkoxy group, an acyl group, an aryl group, and the like. Specific examples of alkylsulfonate include methanesulfonate, ethanesulfonate, butanesulfonate, hexanesulfonate, octanesulfonate, benzylsulfonate, trifluoromethanesulfonate, pentafluoroethanesulfonate, nonafluorobutanesulfonate, and the like. Examples of an aryl group of arylsulfonate include a benzene ring, a naphthalene ring, and an anthracene ring. The benzene ring, the naphthalene ring, and the anthracene ring may have a substituent group, and as the substituent group, a linear or branched alkyl group with 1 to 6 carbon atoms and a cycloalkyl group with 3 to 6 carbon atoms are preferable. Specific examples of the linear or branched alkyl group and cycloalkyl group include methyl, ethyl, n-propyl, isopropyl, n-butyl, i-butyl, t-butyl, n-hexyl, cyclohexyl, and the like. Examples of another substituent group include an alkoxy group with 1 to 6 carbon atoms, a halogen atom, a cyano group, a nitro group, an acyl group, an acyloxy group, and the like.

An amine compound which has a phenoxy group and an ammonium salt compound which has a phenoxy group have a phenoxy group at the end of the opposite side of a nitrogen atom of an alkyl group of the amine compound or the ammonium salt compound. The phenoxy group may have a substituent group. Examples of the substituent group of the phenoxy group include an alkyl group, an alkoxy group, a halogen atom, a cyano group, a nitro group, a carboxyl group, a carbonic acid ester group, a sulfonic acid ester group, an aryl group, an aralkyl group, an acyloxy group, an aryloxy group, and the like. A substituted position of the substituent group may be any of the 2 to 6 positions. The number of the substituent groups may be any number within a range of 1 to 5.

It is preferable to have at least one oxyalkylene group between a phenoxy group and a nitrogen atom. It is preferable to have one or more oxyalkylene groups in the molecule, it is more preferable to have 3 to 9 in the molecule, and it is even more preferable to have 4 to 6 in the molecule. Among oxyalkylene groups, an oxyethylene group (—CH₂CH₂O—) or an oxypropylene group (—CH(CH₃)CH₂O— or —CH₂CH₂CH₂O—) is preferable, and an oxyethylene group is more preferable.

As a sulfonic acid ester group in an amine compound which has a sulfonic acid ester group and an ammonium salt compound which has a sulfonic acid ester group, the sulfonic acid ester group may be any of an alkyl sulfonic acid ester, a cycloalkyl group sulfonic acid ester, and an aryl sulfonic acid ester, and it is preferable that an alkyl group has 1 to 20 carbon atoms in a case of an alkyl sulfonic acid ester, a cycloalkyl group has 3 to 20 carbon atoms in a case of a cycloalkyl sulfonic acid ester, and an aryl group has 6 to 12 carbon atoms in a case of an aryl sulfonic acid ester. The alkyl sulfonic acid ester, the cycloalkyl sulfonic acid ester, and the aryl sulfonic acid ester may have a substituent group and, as the substituent group, a halogen atom, a cyano group, a nitro group, a carboxyl group, a carbonic acid ester group, and a sulfonic acid ester group are preferable.

It is preferable to have at least one oxyalkylene group between a sulfonic acid ester group and a nitrogen atom. It is preferable to have one or more oxyalkylene groups in the molecule, it is more preferable to have 3 to 9 in the molecule, and it is even more preferable to have 4 to 6 in the molecule. Among oxyalkylene groups, an oxyethylene group (—CH₂CH₂O—) or an oxypropylene group (—CH(CH₃)CH₂O— or —CH₂CH₂CH₂O—) is preferable and an oxyethylene group is more preferable.

In addition, the compounds below are also preferable as the basic compound.

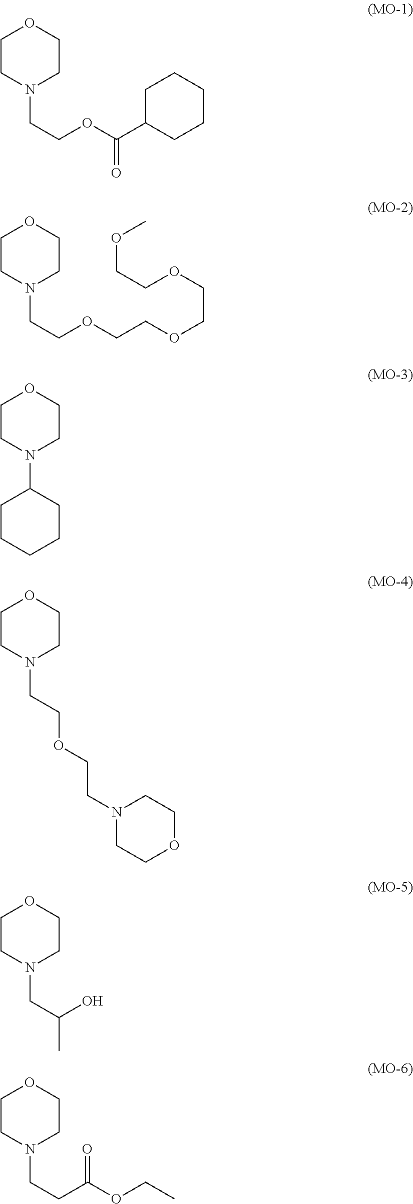

As a basic compound, it is also possible to use compounds which are described in paragraphs 0180 to 0225 of JP2011-22560A, paragraphs 0218 and 0219 of JP2012-137735, and paragraphs 0416 to 0438 of the international public pamphlet WO2011/158687A and the like, other than the compounds described above. The basic compound may be a basic compound or an ammonium salt compound where the basicity decreases due to irradiation with actinic rays or radiation.

The basic compounds may be used as one type individually or may be used in a combination of two or more types.

The composition of the present invention may or may not contain a basic compound; however, when contained, the content ratio of the basic compound is generally 0.001 mass % to 10 mass % and preferably 0.01 mass % to 5 mass % using the solid content of the actinic-ray-sensitive or radiation-sensitive resin composition as a reference.

The usage ratio of the acid generating agent (including the acid generating agent (A′)) and a basic compound in the composition is preferably acid generating agent/basic compound (molar ratio)=2.5 to 300. That is, it is preferable that the molar ratio is 2.5 or more from the point of view of the sensitivity and resolution and 300 or less is preferable from the point of the view of suppressing decreases in the resolution due to the resist pattern becoming thick due to the passing of time to the heating step after exposure. The acid generating agent/basic compound (molar ratio) is more preferably 5.0 to 200 and even more preferably 7.0 to 150.

With respect to the low molecular compound (C) shown in [4] below, it is preferable to use the basic compounds in a molar ratio of low molecular compound (C)/basic compound=100/0 to 10/90, more preferably 100/0 to 30/70, and particularly preferably 100/0 to 50/50.

Here, a low molecular compound (C) which has a nitrogen atom and a group which leaves by an action of an acid which will be described below is not included in the basic compound.

[4] A Low Molecular Compound which has a Nitrogen Atom and a Group which Leaves by an Action of an Acid

The composition of the present invention may contain a compound (also referred to below as “compound (C)”) which has a nitrogen atom and a group which leaves by an action of an acid.

The group which leaves by an action of an acid is not particularly limited; however, an acetal group, a carbonate group, a carbamate group, a tertiary ester group, a tertiary hydroxyl group, and a hemiaminal ether group are preferable, and a carbamate group and a hemiaminal ether group are particularly preferable.

The molecular weight of the compound (C) which has a group which leaves by an action of an acid is preferably 100 to 1000, more preferably 100 to 700, and particularly preferably 100 to 500.

The compound (C) is preferably an amine derivative which has a group which leaves by an action of an acid on a nitrogen atom.

The compound (c) may have a carbamate group which has a protective group on a nitrogen atom. It is possible to represent the protective group which configures a carbamate group by General Formula (d-1) below.

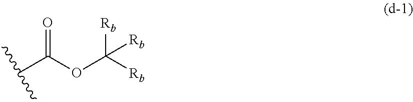

In General Formula (d-1), Rb each independently represents a hydrogen atom, an alkyl group (preferably with 1 to 10 carbon atoms), a cycloalkyl group (preferably with 3 to 30 carbon atoms), an aryl group (preferably with 3 to 30 carbon atoms), an aralkyl group (preferably with 1 to 10 carbon atoms), or an alkoxyalkyl group (preferably 1 to 10 carbon atoms). Rb may form a ring by linking with each other.

An alkyl group, a cycloalkyl group, an aryl group, and an aralkyl group shown by Rb may be substituted with a functional group such as a hydroxyl group, a cyano group, an amino group, a pyrrolidino group, a piperidino group, a morpholino group, and an oxo group, an alkoxy group, and a halogen atom. The same applies to the alkoxyalkyl group shown by Rb.

Examples of an alkyl group, a cycloalkyl group, an aryl group, and an aralkyl group of the Rb (the alkyl group, the cycloalkyl group, the aryl group, and the aralkyl group may be substituted with the functional group, the alkoxy group, and the halogen atom described above) include a linear or branched group which is derived from alkanes such as methane, ethane, propane, butane, pentane, hexane, heptane, octane, nonane, decane, undecane, and dodecane, a group which substitutes a group which is derived from the alkane with, for example, one or more types or one or more of cycloalkyl groups such as a cyclobutyl group, a cyclopentyl group, and a cyclohexyl group, a group which is derived from a cycloalkane such as cyclobutane, cyclopentane, cyclohexane, cycloheptane, cyclooctane, norbornane, adamantane, and noradamantane, a group which substitutes a group which is derived from the cycloalkane with, for example, one or more types or one or more of linear or branched alkyl groups such as a methyl group, an ethyl group, an n-propyl group, an i-propyl group, an n-butyl group, a 2-methylpropyl group, a 1-methylpropyl group, and a t-butyl group, a group which is derived from an aromatic compound such as benzene, naphthalene, and anthracene, a group which substitutes a group which is derived from the aromatic compounds with, for example, one or more types or one or more of linear or branched alkyl groups such as a methyl-group, an ethyl group, an n-propyl group, an i-propyl group, an n-butyl group, a 2-methylpropyl group, a 1-methylpropyl group, and a t-butyl group, a group which is derived from a heterocyclic compound such as pyrrolidine, piperidine, morpholine, tetrahydrofuran, tetrahydropyran, indole, indoline, quinoline, perhydroquinoline, indazole, and benzimidazole, a group which substitutes a group which is derived from the heterocyclic compound with one or more types or one or more of linear or branched alkyl groups or groups which are derived from an aromatic compound, a group and the like which substitute a group which is derived from linear or branched alkane and/or a group which is derived from cycloalkane with one or more types or one or more of groups which are derived from an aromatic compound such as a phenyl group, a naphthyl group, an anthracenyl group, or the like, or a group where the substituent group is substituted with a functional group such as a hydroxyl group, a cyano group, an amino group, a pyrrolidino group, a piperidino group, a morpholino group, and an oxo group, and the like.

Rb is preferably a linear or branched alkyl group, a cycloalkyl group, and an aryl group. A linear or branched alkyl group and a cycloalkyl group are more preferable.

Examples of a ring which two Rb form by linking with each other include an alicyclic hydrocarbon group, an aromatic hydrocarbon group, a heterocyclic hydrocarbon group, or derivatives thereof, and the like.

Examples of a specific structure of a group which is represented by General Formula (d-1) include the structures disclosed in paragraph 0466 of US2012/0135348A1; however, the present invention is not limited thereto.

It is particularly preferable that the compound (C) has a structure which is represented by General Formula (6) below.

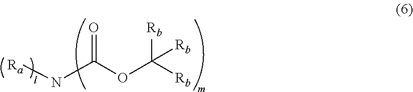

In General Formula (6), Ra represents a hydrogen atom, an alkyl group, a cycloalkyl group, an aryl group, or an aralkyl group. When I is 2, two of Ra may be the same or may be different and the two Ra may form a hetero ring with a nitrogen atom in the formula by linking with each other. The hetero ring may include a hetero atom other than the nitrogen atom in the formula.

Rb is the same as Rb in General Formula (d-1) and the preferable examples thereof are also the same.

I represents an integer of 0 to 2 and m represents an integer of 1 to 3 and I+m=3 is satisfied.

In General Formula (6), an alkyl group, a cycloalkyl group, an aryl group, and an aralkyl group as Ra may be substituted with the same groups as the groups described above as the groups with which an alkyl group, a cycloalkyl group, an aryl group, and an aralkyl group as Rb may be substituted.

Specific examples of an alkyl group, a cycloalkyl group, an aryl group, and an aralkyl group as Ra (the alkyl group, the cycloalkyl group, the aryl group, and the aralkyl group may be substituted with the groups described above) include the same groups as the specific examples described above with regard to Rb.

In addition, a hetero ring which the Ra form by linking with each other preferably has 20 or fewer carbon atoms and examples thereof include a group which is derived from a heterocyclic compound such as pyrrolidine, piperidine, morpholine, 1,4,5,6-tetrahydropyrimidine, 1,2,3,4-tetrahydroquinoline, 1,2,3,6-tetrahydropyridine, homopiperazine, 4-azabenzimidazole, benzotriazole, 5-azabenzotriazole, 1H-1,2,3-triazole, 1,4,7-triazacyclononane, tetrazole, 7-azaindole, indazole, benzimidazole, imidazo[1,2-a]pyridine, (1S,4S)-(+)-2,5-diazabicyclo[2.2.1]heptane, 1,5,7-triazabicyclo[4.4.0]dec-5-en, indole, indoline, 1,2,3,4-tetrahydroquinoxaline, perhydroquinoline, and 1,5,9-triazacyclododecane, a group which substitutes a group which is derived from these heterocyclic compounds with one or more types or one or more of functional groups such as a linear or branched group which is derived from alkane, a group which is derived from cycloalkane, a group which is derived from an aromatic compound, a group which is derived from a heterocyclic compound, a hydroxyl group, a cyano group, an amino group, a pyrrolidino group, a piperidino group, a morpholino group, and an oxo group.

Specific examples of particularly preferable compounds (C) in the present invention include the compounds disclosed in paragraph 0475 of US2012/0135348A1; however, the present invention is not limited thereto.

It is possible to synthesize a compound which is represented by General Formula (6) based on JP2007-298569A, JP2009-199021A, and the like.

In the present invention, it is possible to use a low molecular compound (C) which has a group which leaves by an action of an acid on a nitrogen atom as one type individually or in a combination of two or more types.

The content of the compound (C) in the actinic-ray-sensitive or radiation-sensitive resin composition of the present invention is preferably 0.001 mass % to 20 mass %, more preferably 0.001 mass % to 10 mass %, and even more preferably 0.01 mass % to 5 mass % using the total solid content of the composition as a reference.

[5] Basic Compound where Basicity Decreases or Disappears Due to Irradiation with Actinic Rays or Radiation

The composition of the present invention may contain a basic compound where basicity decreases or disappears due to irradiation with actinic rays or radiation. Examples of basic compounds where basicity decreases or disappears due to irradiation with actinic rays or radiation include the compounds described on pages 171 to 188 of WO2011/083872A. In addition, examples of basic compounds where basicity decreases or disappears due to irradiation with actinic rays or radiation include a sulfonium salt compound which is shown by Formula (a1) below and an iodonium salt compound which is represented by Formula (a2) below.

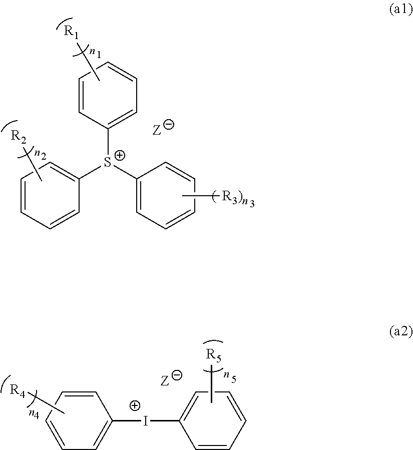

In Formula (a1) and Formula (a2) described above, R₁ to R₅ are each independently a hydrogen atom, an alkyl group, a cycloalkyl group, an alkoxy group, a hydroxyl group, or a halogen atom. Z⁻ is a counter-anion and for example, is an anion which is represented by OH—, R—COO⁻, R—SO₃⁻, or by Formula (a3) below. Here, R is an alkyl group or an aryl group and R may have a substituent group. n₁ to n₅ each independently represents an integer of 0 to 5.

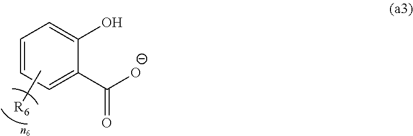

In Formula (a3) described above, R₆ represents a substituent group and n₆ is an integer of 0 to 4.

Examples of compounds which are represented by Formula (a1) and Formula (a2) include a compound which is represented by the structural formula below.

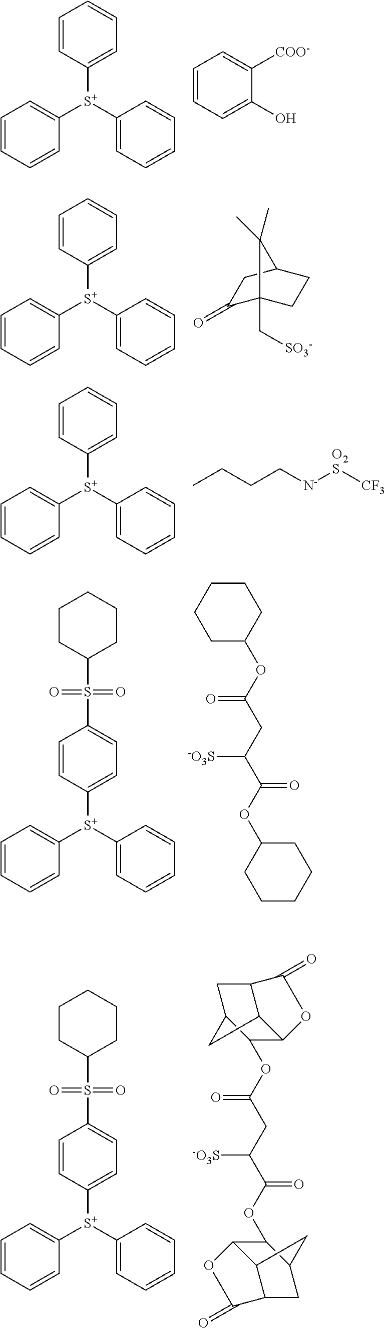

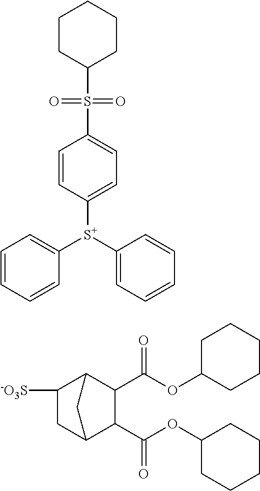

[6] Hydrophobic Resin (D)

The actinic-ray-sensitive or radiation-sensitive resin composition according to the present invention may contain a hydrophobic resin (also referred to below as “hydrophobic resin (D)” or simply “resin (D)), in particular, when applied to liquid immersion exposure. Here, it is preferable that the hydrophobic resin (D) is different from the resin (B).

Due to this, the hydrophobic resin (D) is unevenly distributed in a film surface layer and, in a case where the liquid immersion liquid is water, it is possible to improve the static/dynamic contact angle of a resist film surface with respect to the water and improve the liquid immersion liquid conformance.

The hydrophobic resin (D) is preferably designed so as to be unevenly distributed on an interface as described above; however, unlike a surfactant, it is not necessary to have a hydrophilic group in the molecule, and the resin may or may not contribute to the even mixing of polar/nonpolar substances.

The hydrophobic resin (D) preferably has one or more types of any of a “fluorine atom”, a “silicon atom”, and a “CH₃ partial structure which is contained in a side chain portion of a resin” from the point of view of uneven distribution on the film surface layer, and more preferably has two or more types.

In a case where the hydrophobic resin (D) includes a fluorine atom and/or a silicon atom, the fluorine atom and/or the silicon atom described above in the hydrophobic resin (D) may be included in the main chain of a resin or may be included in a side chain.

In a case where the hydrophobic resin (D) includes a fluorine atom, it is preferably a resin which has an alkyl group which has a fluorine atom, a cycloalkyl group which has a fluorine atom, or an aryl group which has a fluorine atom as a partial structure which has a fluorine atom.

An alkyl group which has a fluorine atom (preferably with 1 to 10 carbon atoms, more preferably with 1 to 4 carbon atoms) is a linear or branched alkyl group where at least one hydrogen atom is substituted with a fluorine atom, and may further have a substituent group other than a fluorine atom.

A cycloalkyl group which has a fluorine atom is a monocyclic or polycyclic cycloalkyl group where at least one hydrogen atom is substituted with a fluorine atom and may further have a substituent group other than a fluorine atom.

Examples of an aryl group which has a fluorine atom include aryl groups where at least one hydrogen atom of an aryl group such as a phenyl group or a naphthyl group is substituted with a fluorine atom, and the aryl group may further have a substituent group other than a fluorine atom.

Preferable examples of an alkyl group which has a fluorine atom, a cycloalkyl group which has a fluorine atom, and an aryl group which has a fluorine atom include groups which are represented by General Formulae (F2) to (F4) below; however, the present invention is not limited thereto.

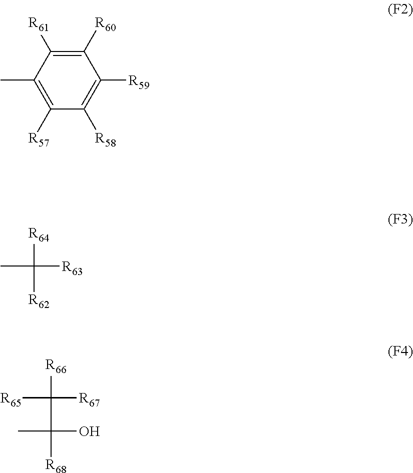

In General Formulae (F2) to (F4), R₅₇ to R₆₈ each independently represents a hydrogen atom, a fluorine atom, or an alkyl group (linear or branched). However, at least one of R₅₇ to R₆₁, at least one of R₆₂ to R₆₄, and at least one of R₆₅ to R₆₈ each independently represents a fluorine atom or an alkyl group (preferably with 1 to 4 carbon atoms) where at least one hydrogen atom is substituted with a fluorine atom.

R₅₇ to R₆₁ and R₆₅ to R₆₇ are preferably all fluorine atoms. R₆₂, R₆₃, and R₆₈ are preferably an alkyl group (preferably with 1 to 4 carbon atoms) where at least one hydrogen atom is substituted with a fluorine atom, and more preferably a perfluoroalkyl group with 1 to 4 carbon atoms. R₆₂ and R₆₃ may form a ring by linking with each other.

Specific examples of a group which is represented by General Formula (F2) include a p-fluorophenyl group, a pentafluorophenyl group, a 3,5-di(trifluoromethyl) phenyl group, and the like.

Specific examples of a group which is represented by General Formula (F3) include a trifluoromethyl group, a pentafluoropropyl group, a pentafluoroethyl group, a heptafluorobutyl group, a hexafluoroisopropyl group, a heptafluoroisopropyl group, a hexafluoro (2-methyl) isopropyl group, a nonafluorobutyl group, an octafluoroisobutyl group, a nonafluorohexyl group, a nonafluoro-t-butyl group, a perfluoroisopentyl group, a perfluorooctyl group, a perfluoro (trimethyl) hexyl group, a 2,2,3,3-tetrafluorocyclobutyl group, a perfluorocyclohexyl group, and the like. A hexafluoroisopropyl group, a heptafluoroisopropyl group, a hexafluoro (2-methyl) isopropyl group, an octafluoroisobutyl group, a nonafluoro-t-butyl group, and a perfluoroisopentyl are preferable, and a hexafluoroisopropyl group and a heptafluoroisopropyl group are more preferable.

Specific examples of a group which is represented by General Formula (F4) include —C(CF₃)₂OH—, —C(C₂F₅)₂OH—, —C(CF₃)(CH₃)OH, —CH(CF₃)OH, and the like, and —C(CF₃)₂OH— is preferable.

A partial structure which includes a fluorine atom may be directly bonded with the main chain and may be further bonded with a main chain via a group which is selected from a group formed of an alkylene group, a phenylene group, an ether bond, a thioether bond, a carbonyl group, an ester bond, an amide bond, a urethane bond, and a ureylene bond, or a group where two or more thereof are combined.

Below, specific examples of a repeating unit which has a fluorine atom will be shown; however, the present invention is not limited thereto.

In the specific examples, X₁ represents a hydrogen atom, —CH₃, —F, or —CF₃. X₂ represents —F or —CF₃.

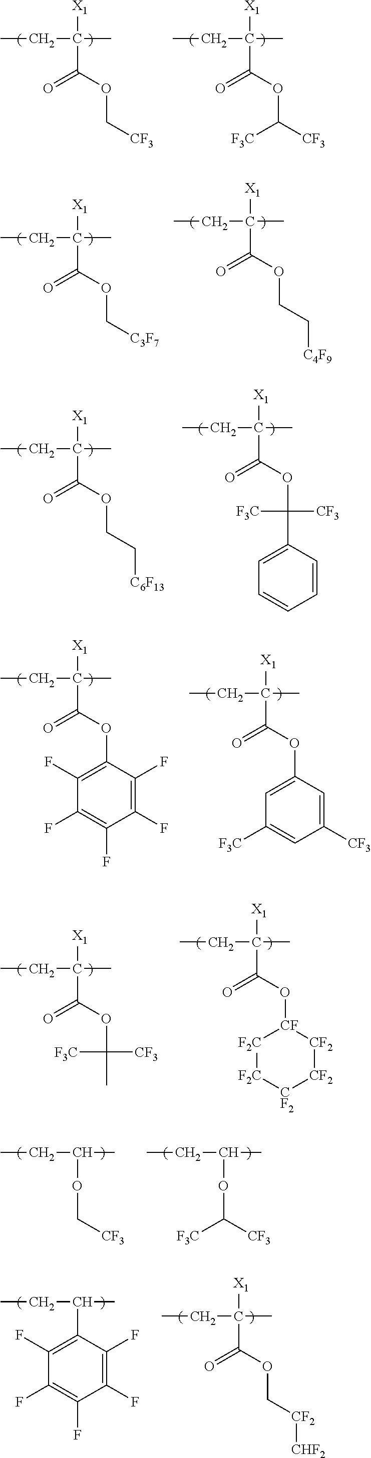

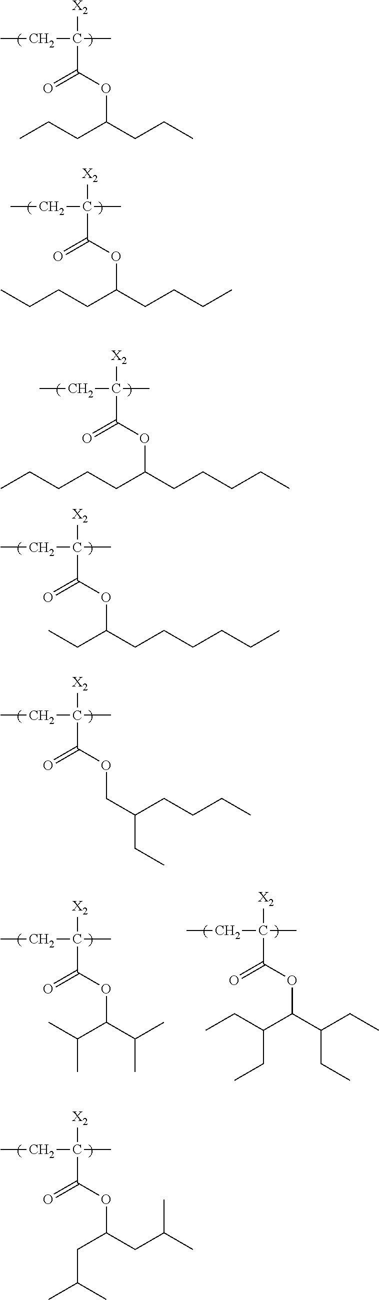

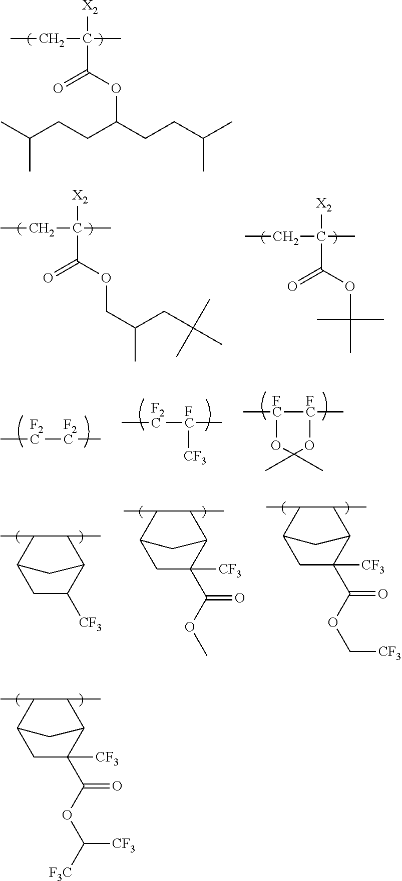

The hydrophobic resin (D) may contain a silicon atom. The hydrophobic resin (D) is preferably a resin which has an alkylsilyl structure (preferably a trialkylsilyl group) or a cyclic siloxane structure as a partial structure which has a silicon atom.

Specific examples of the alkylsilyl structure or the cyclic siloxane structure include a group which is represented by General Formulae (CS-1) to (CS-3) and the like.

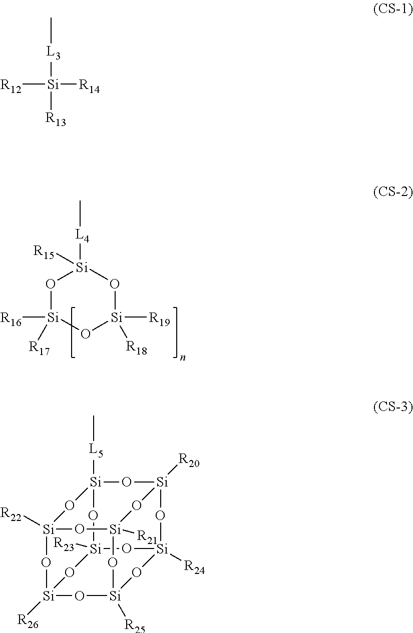

In General Formulae (CS-1) to (CS-3), R₁₂ to R₂₆ each independently represents a linear or branched alkyl group (preferably 1 to 20 carbon atoms) or a cycloalkyl group (preferably 3 to 20 carbon atoms).

L₃ to L₅ represent a single bond or a divalent linking group. Examples of a divalent linking group include one, or a combination (the total number of carbon atoms is preferably 12 or less) of two or more, which are selected from a group formed of an alkylene group, a phenylene group, an ether bond, a thioether bond, a carbonyl group, an ester bond, an amide bond, a urethane bond, and a urea bond.

n represents an integer of 1 to 5. n is preferably an integer of 2 to 4.

In addition, as described above, it is also preferable that the hydrophobic resin (D) includes a CH₃ partial structure in a side chain portion.

Here, the CH₃ partial structure of the side chain portion in the resin (D) includes CH₃ partial structures of an ethyl group, a propyl group, and the like.

On the other hand, a methyl group which is directly bonded with a main chain of the resin (D) (for example an α-methyl group of a repeating unit which has a methacrylic acid structure) is not included in the CH₃ partial structure in the present invention since the contribution to the surface uneven distribution of the resin (D) due to the influence of the main chain is small.

In more detail, in a case where the resin (D) includes a repeating unit which is derived from a monomer which has a polymerizable site which has a carbon-carbon double bond such as a repeating unit which is represented by General Formula (M) below, and in a case where R₁₁ to R₁₄ are CH₃ “itself”, the CH₃ is not included in the CH₃ partial structure of the side chain portion in the present invention.

On the other hand, a CH₃ partial structure which is present via a certain atom from a C—C main chain corresponds to the CH₃ partial structure in the present invention. For example, in a case where R₁₁ is an ethyl group (CH₂CH₃), the resin (D) has “one” CH₃ partial structure in the present invention.

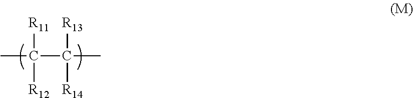

In General Formula (M) described above, R₁₁ to R₁₄ each independently represents a side chain portion.

Examples of R₁₁ to R₁₄ in the side chain portion include a hydrogen atom, a monovalent organic group, and the like.

Examples of a monovalent organic group with regard to R₁₁ to R₁₄ include an alkyl group, a cycloalkyl group, an aryl group, an alkyloxycarbonyl group, a cycloalkyloxycarbonyl group, an aryloxycarbonyl group, an alkylaminocarbonyl group, a cycloalkylaminocarbonyl group, an arylaminocarbonyl group, and the like, and the groups may further have a substituent group.

The hydrophobic resin (D) is preferably a resin which has a repeating unit which has a CH₃ partial structure in a side chain portion, and more preferably has at least one type of repeating unit (x) out of repeating units which are represented by General Formula (II) below and repeating units which are represented by General Formula (III) below as the repeating unit.

Detailed description will be given below of a repeating unit which is represented by General Formula (II).

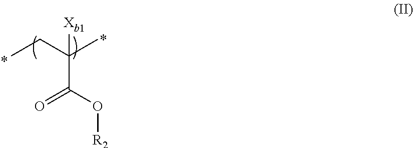

In General Formula (II) described above, X_{b1} represents a hydrogen atom, an alkyl group, a cyano group, or a halogen atom, and R₂ represents an organic group which has one or more CH₃ partial structures and is stable with respect to acid. Here, in more detail, it is preferable that an organic group which is stable with respect to acid is an organic group which does not have the “acid-decomposable group” which is described in the resin (B).

An alkyl group of X_{b1} preferably has 1 to 4 carbon atoms and examples thereof include a methyl group, an ethyl group, a propyl group, a hydroxymethyl group, a trifluoromethyl group, and the like; however, a methyl group is preferable.

X_{b1} is preferably a hydrogen atom or a methyl group.

Examples of R₂ include an alkyl group, a cycloalkyl group, an alkenyl group, a cycloalkenyl group, an aryl group, and an aralkyl group which have one or more CH₃ partial structures. The cycloalkyl group, the alkenyl group, the cycloalkenyl group, the aryl group, and the aralkyl group described above may further have an alkyl group as a substituent group.

R₂ is preferably an alkyl group or an alkyl substituted cycloalkyl group which has one or more CH₃ partial structures.

An organic group which has one or more CH₃ partial structures and is stable to acid as R₂ preferably has 2 or more to 10 or less CH₃ partial structures, and more preferably 2 or more to 8 or less.

As an alkyl group which has one or more CH₃ partial structures in R₂, a branched alkyl group with 3 to 20 carbon atoms is preferable. Specific examples of a preferable alkyl group include an isopropyl group, an isobutyl group, a 3-pentyl group, a 2-methyl-3-butyl group, a 3-hexyl group, a 2-methyl-3-pentyl group, a 3-methyl-4-hexyl group, a 3,5-dimethyl-4-pentyl group, an isooctyl group, 2,4,4-trimethylpentyl group, a 2-ethylhexyl group, 2,6-dimethylheptyl group, a 1,5-dimethyl-3-heptyl group, a 2,3,5,7-tetramethyl-4-heptyl group, and the like. An isobutyl group, a t-butyl group, a 2-methyl-3-butyl group, a 2-methyl-3-pentyl group, a 3-methyl-4-hexyl group, 3,5-dimethyl-4-pentyl group, a 2,4,4-trimethylpentyl group, a 2-ethylhexyl group, a 2,6-dimethylheptyl group, a 1,5-dimethyl-3-heptyl group, and a 2,3,5,7-tetramethyl-4-heptyl group are more preferable.

A cycloalkyl group which has one or more CH₃ partial structures in R₂ may be monocyclic or may be polycyclic. In detail, examples thereof include a group which has a monocyclo, bicyclo, tricyclo, tetracyclo structure and the like with 5 or more carbon atoms. The number of carbon atoms is preferably 6 to 30 and the number of carbon atoms is particularly preferably 7 to 25. Examples of a preferable cycloalkyl group include an adamantyl group, a noradamantyl group, a decalin residue, a tricyclodecanyl group, a tetracyclododecanyl group, a norbornyl group, a cedrol group, a cyclopentyl group, a cyclohexyl group, a cycloheptyl group, a cyclooctyl group, a cyclodecanyl group, and a cyclododecanyl group. More preferable examples include an adamantyl group, a norbornyl group, a cyclohexyl group, a cyclopentyl group, a tetracyclododecanyl group, and a tricyclodecanyl group. A norbornyl group, a cyclopentyl group, and a cyclohexyl group are more preferable.

As an alkenyl group which has one or more CH₃ partial structures in R₂, a linear or branched alkenyl group with 1 to 20 carbon atoms is preferable and a branched alkenyl group is more preferable.

As an aryl group which has one or more CH₃ partial structures in R₂, an aryl group with 6 to 20 carbon atoms is preferable, and examples thereof include a phenyl group and a naphthyl group, and a phenyl group is preferable.

As an aralkyl group which has one or more CH₃ partial structures in R₂, an aralkyl group with 7 to 12 carbon atoms is preferable, and examples thereof include a benzyl group, a phenethyl group, a naphthylmethyl group, and the like.

Specific examples of a hydrocarbon group which has two or more CH₃ partial structures in R₂ include an isopropyl group, an isobutyl group, a t-butyl group, a 3-pentyl group, a 2-methyl-3-butyl group, a 3-hexyl group, a 2,3-dimethyl-2-butyl group, a 2-methyl-3-pentyl group, a 3-methyl-4-hexyl group, 3,5-dimethyl-4-pentyl group, an isooctyl group, a 2,4,4-trimethylpentyl group, a 2-ethylhexyl group, a 2-6-dimethylheptyl group, a 1,5-dimethyl-3-heptyl group, a 2,3,5,7-tetramethyl-4-heptyl group, a 3,5-dimethylcyclohexyl group, a 4-isopropylcyclohexyl group, a 4-t-butylcyclohexyl group, an isobornyl group, and the like. An isobutyl group, a t-butyl group, a 2-methyl-3-butyl group, a 2,3-dimethyl-2-butyl group, a 2-methyl-3-pentyl group, a 3-methyl-4-hexyl group, a 3,5-dimethyl-4-pentyl group, a 2,4,4-trimethylpentyl group, a 2-ethylhexyl group, a 2,6-dimethylheptyl group, a 1,5-dimethyl-3-heptyl group, a 2,3,5,7-tetramethyl-4-heptyl group, a 3,5-dimethylcyclohexyl group, a 3,5-di-tert-butylcyclohexyl group, a 4-isopropylcyclohexyl group, a 4-t-butylcyclohexyl group, and an isobornyl group are more preferable.

Preferable specific examples of a repeating unit which is represented by General Formula (II) will be given below. Here, the present invention is not limited thereto.

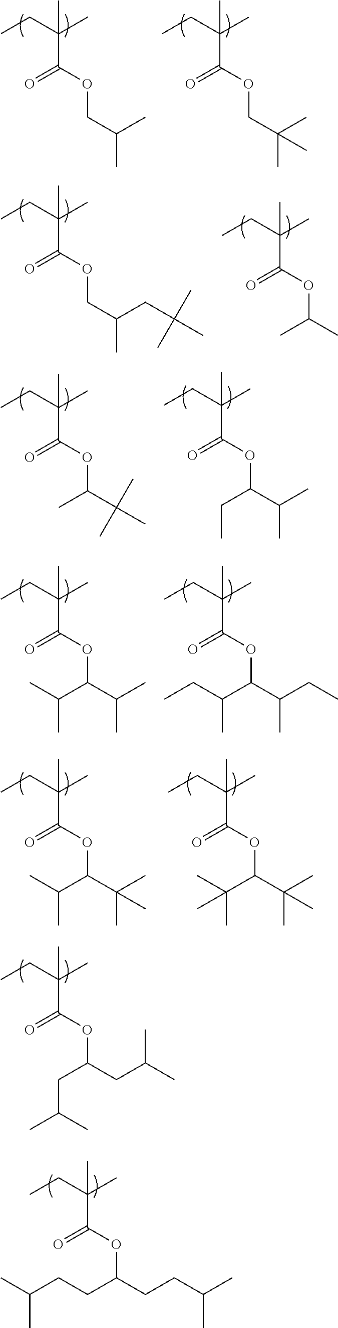

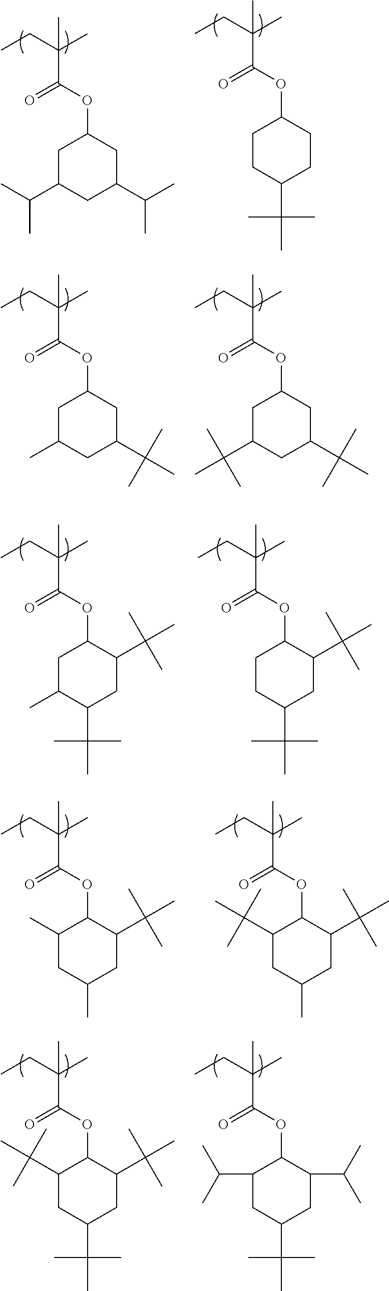

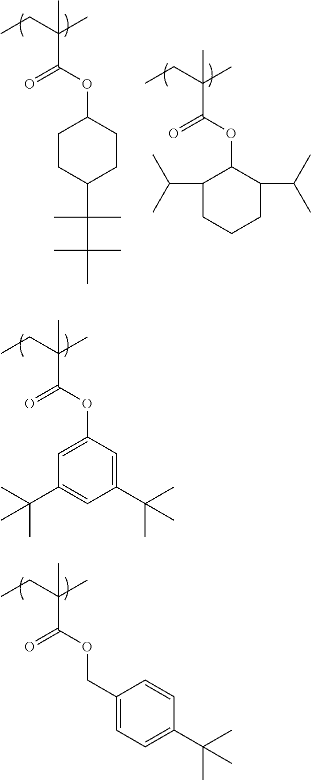

The repeating unit which is represented by General Formula (II) is preferably a (acid non-decomposable) repeating unit which is stable in acid and, specifically, it is preferably a repeating unit which is decomposed due to the action of an acid and which does not have a group which generates a polar group.

Detailed description will be given below of a repeating unit which is represented by General Formula (III).

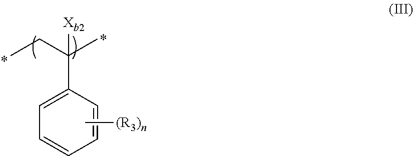

In General Formula (III) described above, X_{b2} represents a hydrogen atom, an alkyl group, a cyano group, or a halogen atom, R₃ represents an organic group which has one or more CH₃ partial structures and is stable with respect to acid, and n represents an integer of 1 to 5.

An alkyl group of X_{b2} is preferably an alkyl group with 1 to 4 carbon atoms and examples thereof include a methyl group, an ethyl group, a propyl group, a hydroxymethyl group, a trifluoromethyl group, and the like.

X_{b2} is preferably a hydrogen atom.

Since R₃ is an organic group which is stable with respect to acid, in more detail, it is preferably an organic group which does not have the “acid-decomposable group” which is described in the resin (B).

Examples of R₃ include an alkyl group which has one or more CH₃ partial structures.

an organic group which has one or more CH₃ partial structures and is stable to acid as R₃ preferably has 1 or more to 10 or less CH₃ partial structures, more preferably 1 or more to 8 or less, and even more preferably 1 or more to 4 or less.

As an alkyl group which has one or more CH₃ partial structures in R₃, a branched alkyl group with 3 to 20 carbon atoms is preferable. Specific examples of a preferable alkyl group include an isopropyl group, an isobutyl group, a 3-pentyl group, a 2-methyl-3-butyl group, a 3-hexyl group, a 2-methyl-3-pentyl group, a 3-methyl-4-hexyl group, a 3,5-dimethyl-4-pentyl group, an isooctyl group, a 2,4,4-trimethylpentyl group, a 2-ethylhexyl group, a 2,6-dimethylheptyl group, a 1,5-dimethyl-3-heptyl group, a 2,3,5,7-tetramethyl-4-heptyl group, and the like. An isobutyl group, a t-butyl group, a 2-methyl-3-butyl group, a 2-methyl-3-pentyl group, a 3-methyl-4-hexyl group, a 3,5-dimethyl-4-pentyl group, a 2,4,4-trimethylpentyl group, a 2-ethylhexyl group, a 2,6-dimethylheptyl group, a 1,5-dimethyl-3-heptyl group, and a 2,3,5,7-tetramethyl-4-heptyl group are more preferable.

Specific examples of an alkyl group which has 2 or more CH₃ partial structures in R₃ include an isopropyl group, an isobutyl group, a t-butyl group, a 3-pentyl group, a 2,3-dimethylbutyl group, a 2-methyl-3-butyl group, a 3-hexyl group, a 2-methyl-3-pentyl group, a 3-methyl-4-hexyl group, a 3,5-dimethyl-4-pentyl group, an isooctyl group, a 2,4,4-trimethylpentyl group, a 2-ethylhexyl group, a 2,6-dimethylheptyl group, a 1,5-dimethyl-3-heptyl group, and a 2,3,5,7-tetramethyl-4-heptyl group, and the like. The number of carbon atoms is preferably 5 to 20 and an isopropyl group, a t-butyl group, a 2-methyl-3-butyl group, a 2-methyl-3-pentyl group, a 3-methyl-4-hexyl group, a 3,5-dimethyl-4-pentyl group, a 2,4,4-trimethylpentyl group, a 2-ethylhexyl group, a 2,6-dimethylheptyl group, a 1,5-dimethyl-3-heptyl group, and a 2,3,5,7-tetramethyl-4-heptyl group, and 2,6-dimethylheptyl group are more preferable.

n represents an integer of 1 to 5, more preferably an integer of 1 to 3, and even more preferably 1 or 2.

Preferable specific examples of a repeating unit which is represented by General Formula (III) will be given below. Here, the present invention is not limited thereto.

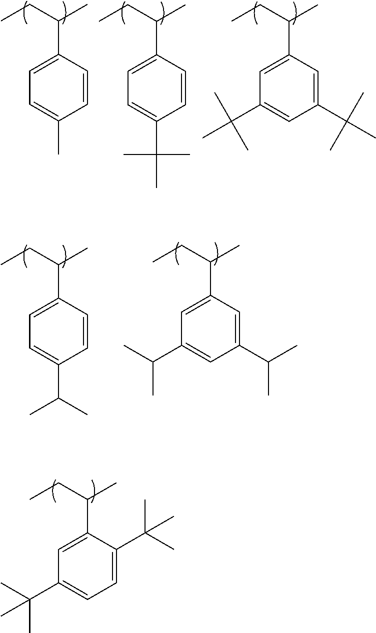

A repeating unit which is represented by General Formula (III) is preferably a (acid non-decomposable) repeating unit which is stable in acid, specifically, a repeating unit which is decomposed due to the action of an acid and does not have a group which generates a polar group is preferable.

In a case where the resin (D) includes a CH₃ partial structure in a side chain portion and, additionally, in a case of not having a fluorine atom or a silicon atom in particular, it is preferable that the content of at least one type of a repeating unit (x) out of the repeating units which are represented by General Formula (II) and the repeating units which are represented by General Formula (III) is 90 mol % or more with respect to all of the repeating units of the resin (C), more preferably 95 mol % or more. The content is generally 100 mol % or less with respect to all of the repeating units of the resin (C).

By the resin (D) containing at least one type of repeating unit (x) out of the repeating units which are represented by General Formula (II) and the repeating units which are represented by General Formula (III) at 90 mol % or more with respect to the all of the repeating units of the resin (D), the surface free energy of the resin (C) increases. As the result thereof, the resin (D) is not easily unevenly distributed on the surface of the resist film and it is possible to reliably improve the static/dynamic contact angle of the resist film with respect to the water and improve the liquid immersion liquid conformance.

In addition, (i) even in a case of including a fluorine atom and/or a silicon atom, or (ii) even in a case of including a CH₃ partial structure in a side chain portion, the hydrophobic resin (D) may have at least one group which is selected from a group of (x) to (z) below.

(x) acid group

(y) group which has a lactone structure, an acid anhydride group, or an acid imide group

(z) group which is decomposed due to the action of an acid

Examples of an acid group (x) include a phenolic hydroxyl group, a carboxylic acid group, a fluorinated alcohol group, a sulfonic acid group, a sulfonamide group, a sulfonylimide group, a (alkylsulfonyl) (alkylcarbonyl)methylene group, a (alkylsulfonyl) (alkylcarbonyl) imide group, a bis(alkylcarbonyl)methylene group, a bis(alkylcarbonyl) imide group, a bis(alkylsulfonyl)methylene group, a bis(alkylsulfonyl) imide group, a tris(alkylcarbonyl)methylene group, a tris(alkylsulfonyl)methylene group, and the like.

Examples of preferable acid groups include a fluorinated alcohol group (preferably hexafluoroisopropanol), a sulfonimide group, and a bis(alkylcarbonyl)methylene group.

Examples of a repeating unit which has an acid group (x) include a repeating unit where an acid group is directly bonded with a main chain of a resin such as a repeating unit by an acrylic acid and a methacrylic acid, or a repeating unit where an acid group is bonded with a main chain of a resin via a bonding group and, additionally, it is also possible to use a polymerization initiator or a chain transfer agent which has an acid group during the polymerization and introduce the polymerization initiator or the chain transfer agent to the end of a polymer chain, and either case is preferable. A repeating unit which has an acid group (x) may have at least either of a fluorine atom or a silicon atom.

The content of the repeating unit which has an acid group (x) is preferably 1 mol % to 50 mol %, more preferably 3 mol % to 35 mol %, and even more preferably 5 mol % to 20 mol % with respect to all of the repeating units in the hydrophobic resin (D).

Specific examples of a repeating unit which has an acid group (x) will be shown below; however, the present invention is not limited thereto. In the formula, Rx represents a hydrogen atom, CH₃, CF₃, or CH₂OH.

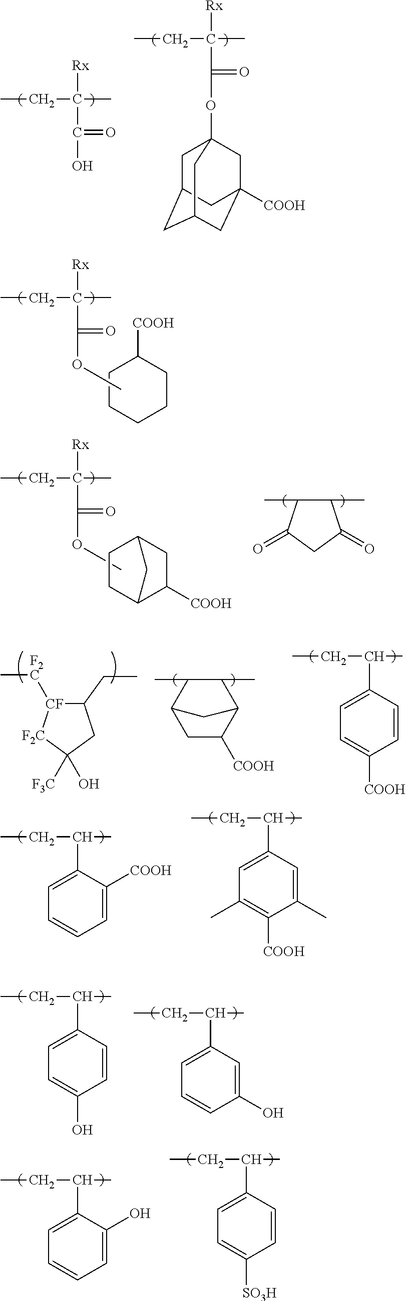

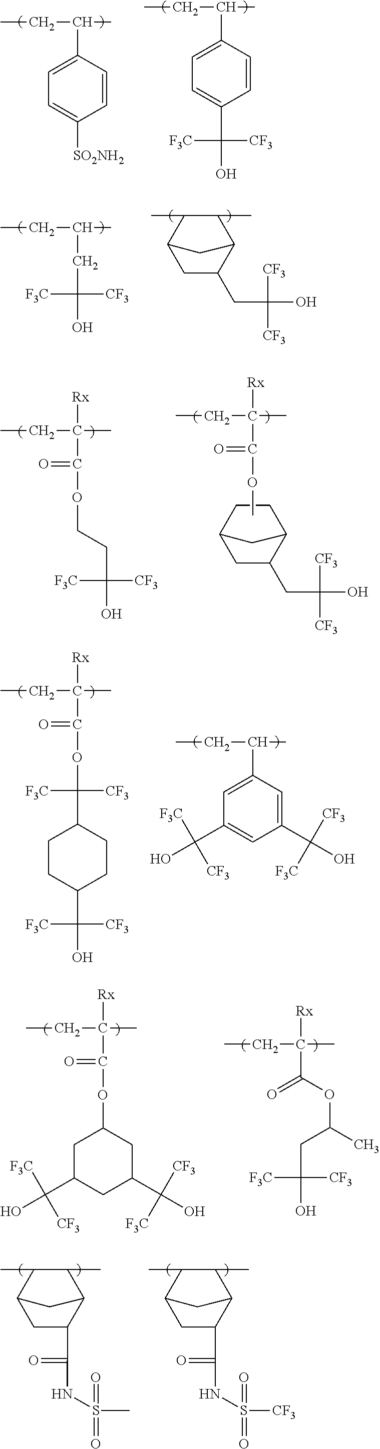

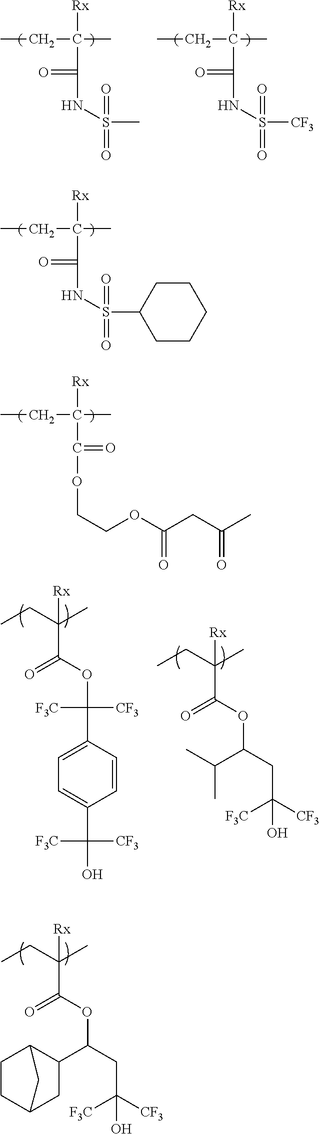

As a group which has a lactone structure, an acid anhydride group, or an acid imide group (y), a group which has a lactone structure is particularly preferable.

The repeating unit which includes the groups is, for example, a repeating unit where the groups are directly bonded with a main chain of a resin such as a repeating unit derived from an acrylic acid ester and a methacrylic acid ester. Alternatively, the repeating unit may be a repeating unit where the groups are bonded with the main chain of a resin via a linking group. Alternatively, for the repeating unit, a polymerization initiator or a chain transfer agent which has the groups may be used during the polymerization and introduced to an end of a resin.

Examples of a repeating unit which has a group which has a lactone structure include the same repeating unit as the repeating unit which has the lactone structure which is previously described in the section of the acid-decomposable resin (the resin (B)).

The content of a repeating unit which has a group which has a lactone structure, an acid anhydride group, or an acid imide group is preferably 1 mol % to 100 mol %, more preferably 3 mol % to 98 mol %, and even more preferably 5 mol % to 95 mol % using all of the repeating units in the hydrophobic resin (D) as a reference.

Examples of a repeating unit which has a group (z) which is decomposed due to the action of an acid in the hydrophobic resin (D) include the same repeating units as the repeating units which have an acid-decomposable group which are given for the resin (B). A repeating unit which has a group (z) which is decomposed due to the action of an acid may have at least either of a fluorine atom or a silicon atom. The content of a repeating unit which has a group (z) which is decomposed due to the action of an acid in the hydrophobic resin (D) is preferably 1 mol % to 80 mol %, more preferably 10 mol % to 80 mol %, and even more preferably 20 mol % to 60 mol % with respect to all of the repeating units in the resin (D).

The hydrophobic resin (D) may further have a repeating unit which is represented by General Formula (III) below.

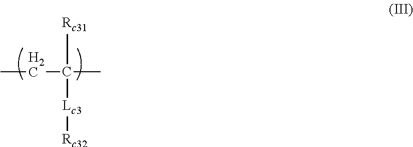

In General Formula (III), R_{c31} represents a hydrogen atom, an alkyl group (which may be substituted with a fluorine atom or the like), a cyano group, or a —CH₂—O-Rac₂ group. In the formula, Rac₂ represents a hydrogen atom, an alkyl group, or an acyl group. R_{c31} is preferably a hydrogen atom, a methyl group, a hydroxymethyl group, and a trifluoromethyl group, and a hydrogen atom and a methyl group are particularly preferable.

R_{c32} represents a group which has an alkyl group, a cycloalkyl group, an alkenyl group, a cycloalkenyl group, or an aryl group. The groups may be substituted with a group which includes a fluorine atom and a silicon atom.

L_{c3} represents a single bond or a divalent linking group.

In General Formula (III), the alkyl group of R_{c32} is preferably a linear or branched alkyl group with 3 to 20 carbon atoms.

The cycloalkyl group is preferably a cycloalkyl group with 3 to 20 carbon atoms.

The alkenyl group is preferably an alkenyl group with 3 to 20 carbon atoms.

The cycloalkenyl group is preferably a cycloalkenyl group with 3 to 20 carbon atoms.

The aryl group is preferably an aryl group with 6 to 20 carbon atoms, a phenyl group and a naphthyl group are more preferable, and these may have a substituent group.

R_{c32} is preferably an unsubstituted alkyl group or an alkyl group which is substituted with a fluorine atom.

A divalent linking group of L_{c3} is preferably an alkylene group (preferably with 1 to 5 carbon atoms), an ether bond, a phenylene group, or an ester bond (a group which is represented by —COO—).

The content of a repeating unit which is represented by General Formula (III) is preferably 1 to 100 mol %, more preferably 10 mol % to 90 mol %, and even more preferably 30 mol % to 70 mol % using all of the repeating units in the hydrophobic resin as a reference.

It is also preferable that the hydrophobic resin (D) further has a repeating unit which is represented by General Formula (CII-AB) below.

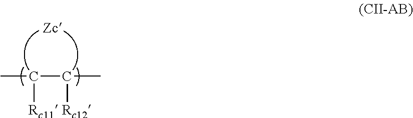

In Formula (CII-AB), Rc₁₁′ and Rc₁₂′ each independently represents a hydrogen atom, a cyano group, a halogen atom, or an alkyl group.

Zc′ represents an atom group which includes two bonded carbon atoms (C—C) and which is for forming an alicyclic structure.

The content of a repeating unit which is represented by General Formula (CII-AB) is preferably 1 to 100 mol %, more preferably 10 mol % to 90 mol %, and even more preferably 30 mol % to 70 mol % using all of the repeating units in the hydrophobic resin as a reference.

Specific examples of a repeating unit which is represented by General Formulae (III) and (CII-AB) will be given below; however, the present invention is not limited thereto. In the formula, Ra represents H, CH₃, CH₂OH, CF₃, or CN.

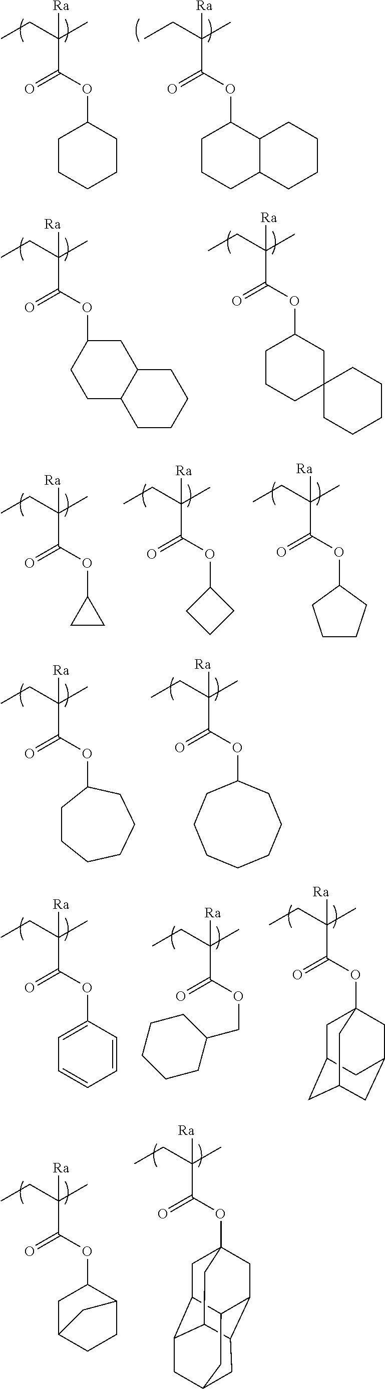

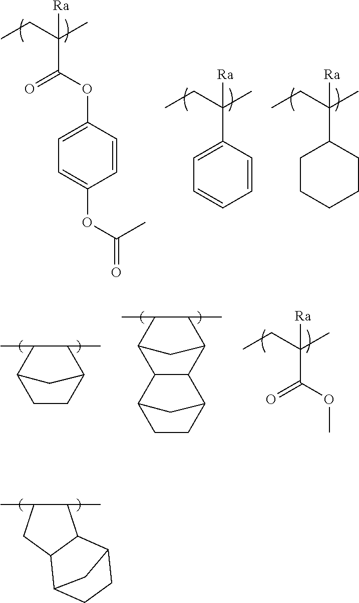

In a case where the hydrophobic resin (D) has fluorine atoms, it is preferable that the content of the fluorine atoms is 5 mass % to 80 mass % and more preferably 10 mass % to 80 mass % with respect to the weight average molecular weight of the hydrophobic resin (D). In addition, it is preferable that a repeating unit which includes a fluorine atom is 10 to 100 mol % and more preferably 30 to 100 mol % in all of the repeating units which are included in the hydrophobic resin (D).

In a case where the hydrophobic resin (D) has silicon atoms, the content of the silicon atoms is preferably 2 mass % to 50 mass % and more preferably 2 mass % to 30 mass % with respect to the weight average molecular weight of the hydrophobic resin (D). In addition, the repeating unit which includes a silicon atom is preferably 10 to 100 mol %, and more preferably 20 to 100 mol % in all of the repeating units which are included in the hydrophobic resin (D).

On the other hand, in particular, in a case where the resin (D) includes a CH₃ partial structure in a side chain portion, a form where the resin (D) substantially does not have a fluorine atom or a silicon atom is also preferable, and in this case, in detail, it is preferable that the content of a repeating unit which has a fluorine atom and a silicon atom is 5 mol % or less, more preferably 3 mol % or less, and even more preferably 1 mol % or less with respect to all of the repeating units in the resin (D), and 0 mol %, that is, not containing a fluorine atom or a silicon atom, is ideal. In addition, it is preferable that the resin (D) is substantially configured only by repeating units which are configured only by atoms which are selected from a carbon atom, an oxygen atom, a hydrogen atom, a nitrogen atom, and a sulfur atom. In more detail, it is preferable that repeating units which are configured only by atoms which are selected from a carbon atom, an oxygen atom, a hydrogen atom, a nitrogen atom, and a sulfur atom are 95 mol % or more, more preferably 97 mol % or more, even more preferably 99 mol % or more, and ideally 100 mol % in the all of the repeating units of the resin (D).

The weight average molecular weight of standard polystyrene conversion of the hydrophobic resin (D) is preferably 1,000 to 100,000, more preferably 1,000 to 50,000, and even more preferably 2,000 to 15,000.

In addition, the hydrophobic resin (D) may be used as one type individually or may be used in a combination of a plurality thereof.

The content in the composition of the hydrophobic resin (D) is preferably 0.01 mass % to 10 mass %, more preferably 0.05 mass % to 8 mass %, and even more preferably 0.1 mass % to 7 mass % with respect to the total solid content in the composition of the present invention.

While the hydrophobic resin (D) naturally does not have many impurities such as metal in the same manner as the resin (B), it is preferable that residual monomers or oligomer components are 0.01 mass % to 5 mass %, more preferably 0.01 mass % to 3 mass %, and even more preferably 0.05 mass % to 1 mass %. Due to this, an actinic-ray-sensitive or radiation-sensitive resin composition is obtained which does not have foreign matter in the liquid or where the sensitivity and the like do not change due to passing of time. In addition, in terms of the resolution, the resist shape, the side wall of a resist pattern, roughness, and the like, the molecular weight distribution (Mw/Mn, also referred to as the dispersity) is preferably within a range of 1 to 5, more preferably 1 to 3, and even more preferably a range of 1 to 2.

It is also possible to use various types of commercial products for the hydrophobic resin (D) and it is also possible to synthesize the hydrophobic resin (D) by a normal method (for example, radical polymerization). Examples of typical synthesis methods include a collective polymerization method which performs polymerization by dissolving a monomer type and an initiator in a solvent and heating, a dropping polymerization method which adds a solution of a monomer type and an initiator dropwise to a heating solvent over 1 to 10 hours, or the like, and the dropping polymerization method is preferable.

The reaction solvent, the polymerization initiator, the reaction conditions (temperature, density, and the like), and the purification method after reaction are the same as for the content which is described for the resin (B); however, in the synthesis of the hydrophobic resin (D), it is preferable that the reaction concentration is 30 mass % to 50 mass %.

Specific examples of the hydrophobic resin (D) will be given below. In addition, in the Tables below, the molar ratio (which corresponds to each repeating unit in order from left), the weight average molecular weight, and the dispersity of the repeating units in each resin will be shown.

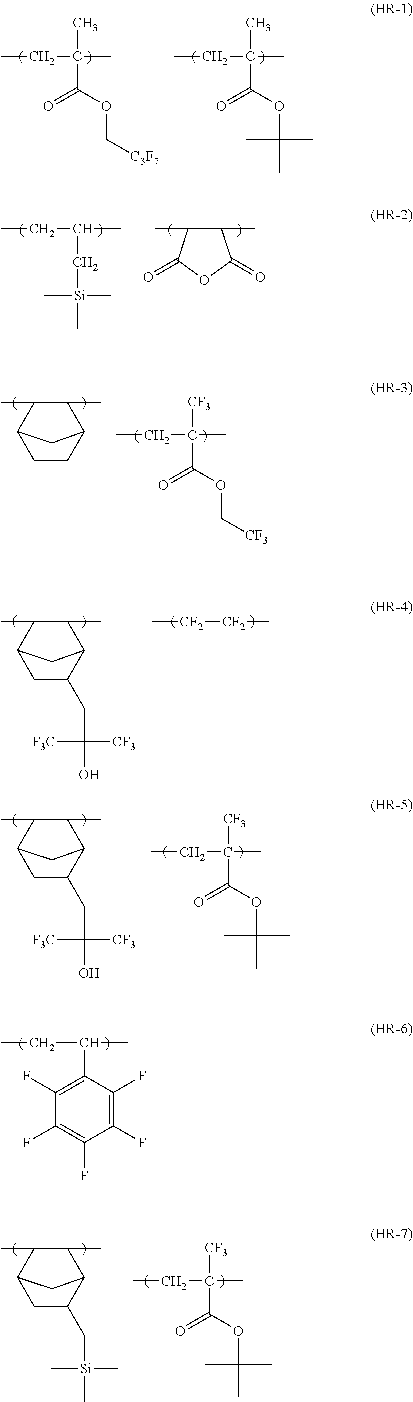

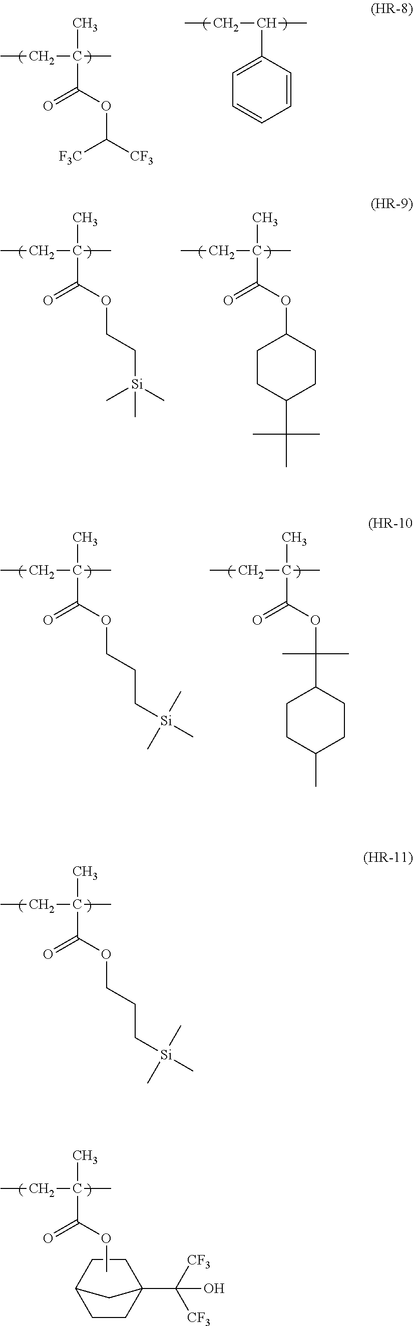

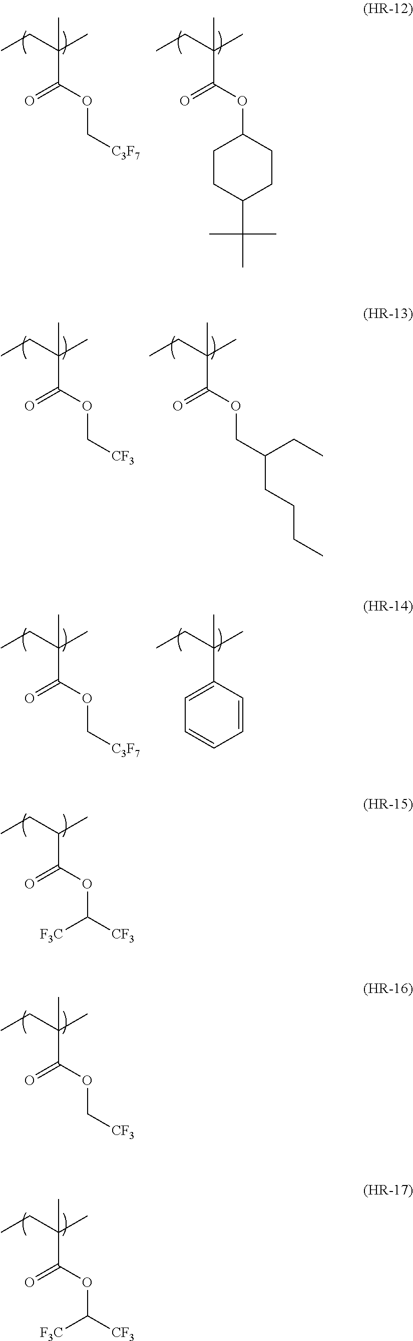

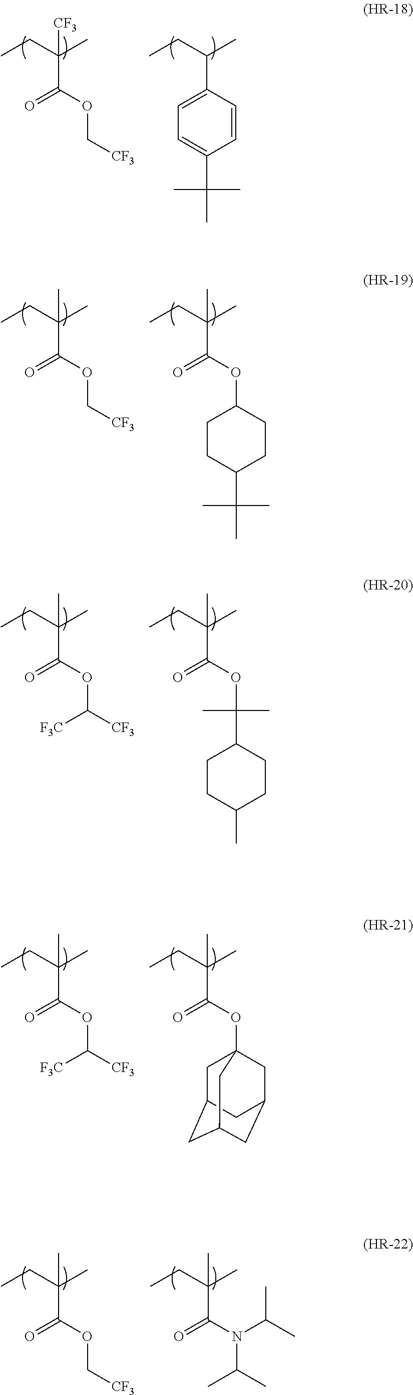

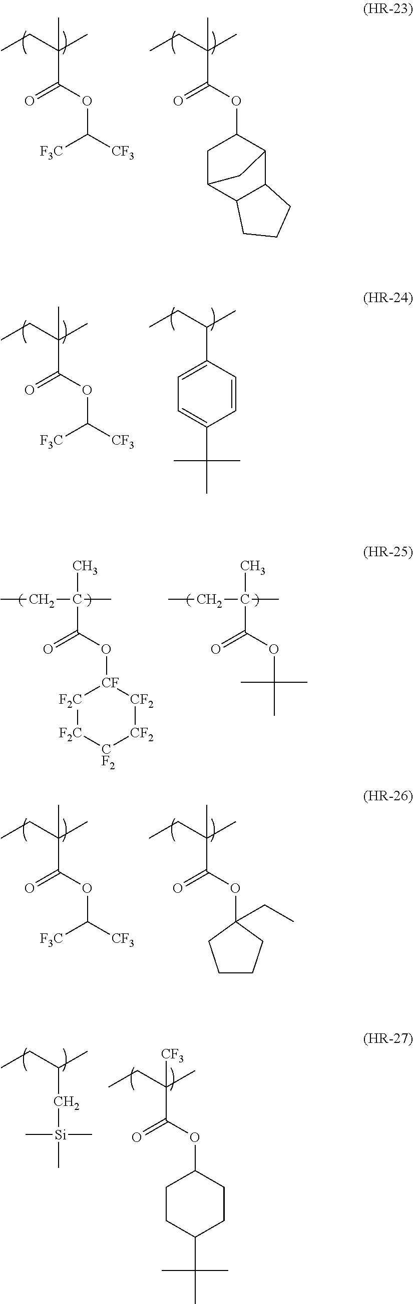

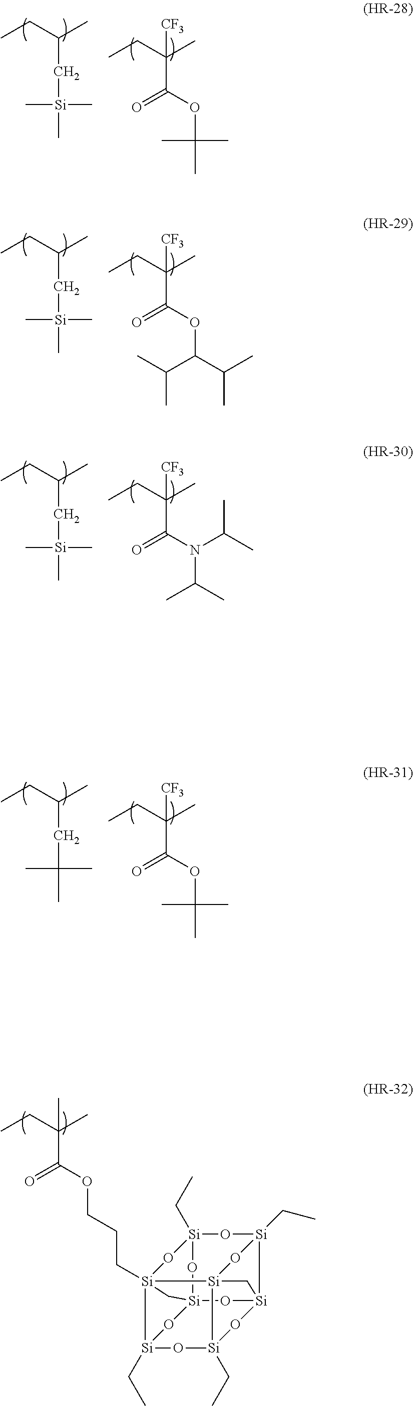

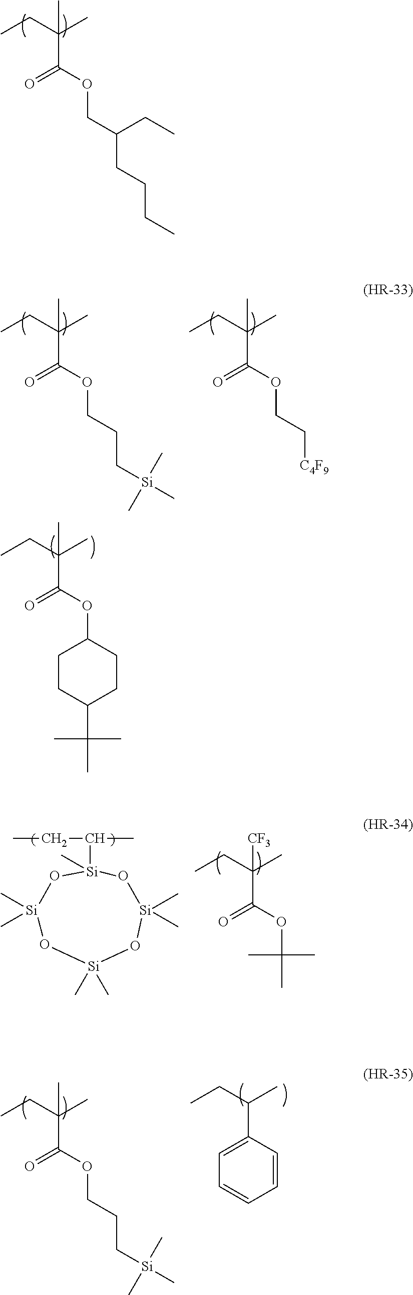

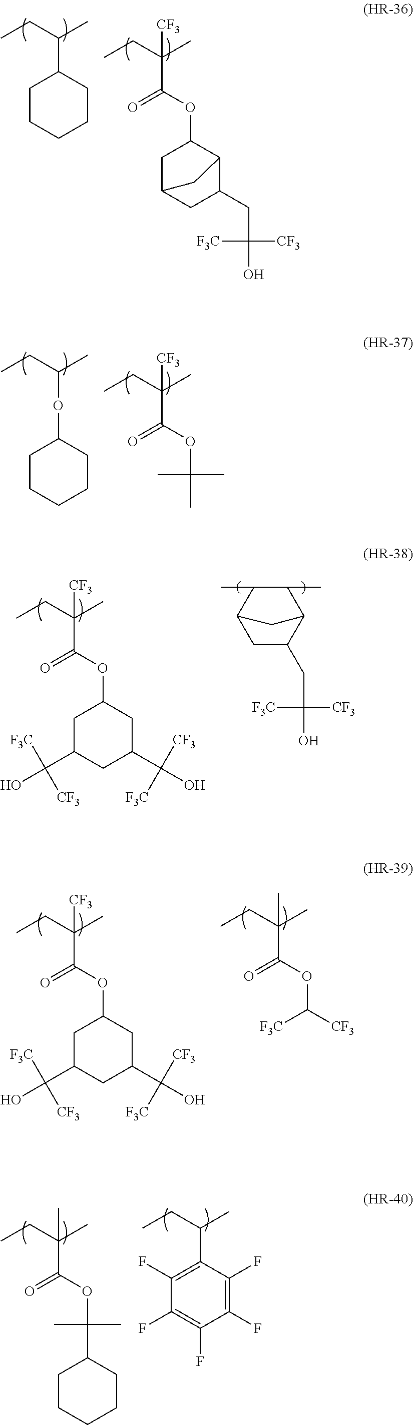

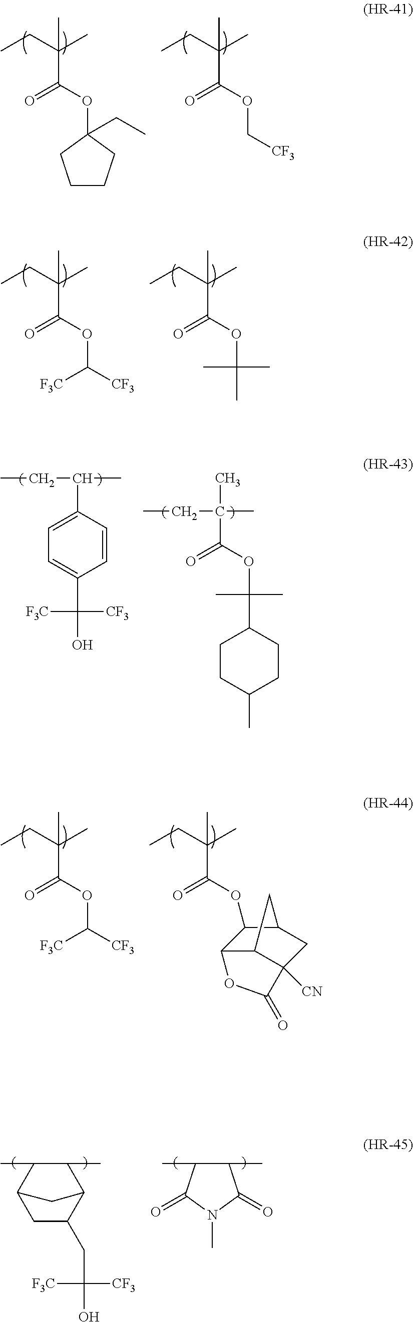

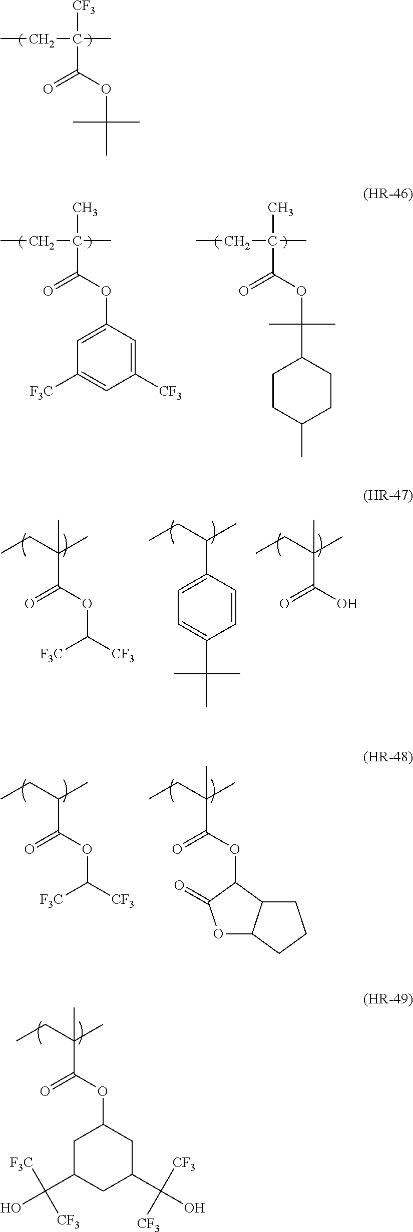

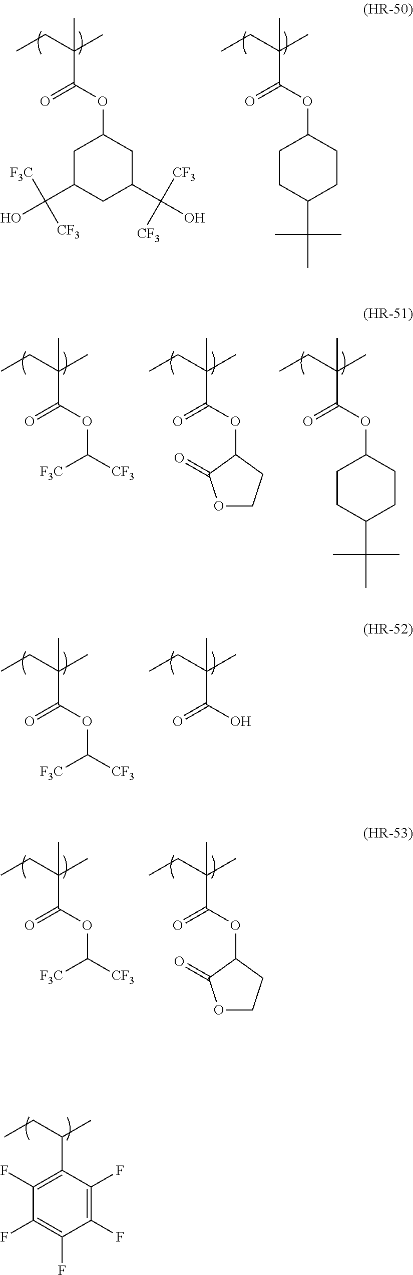

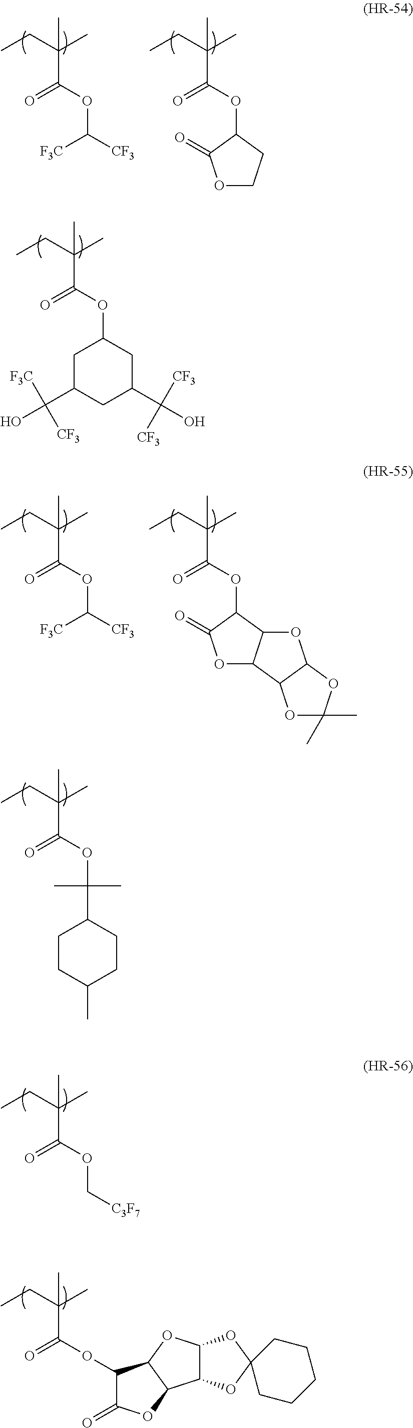

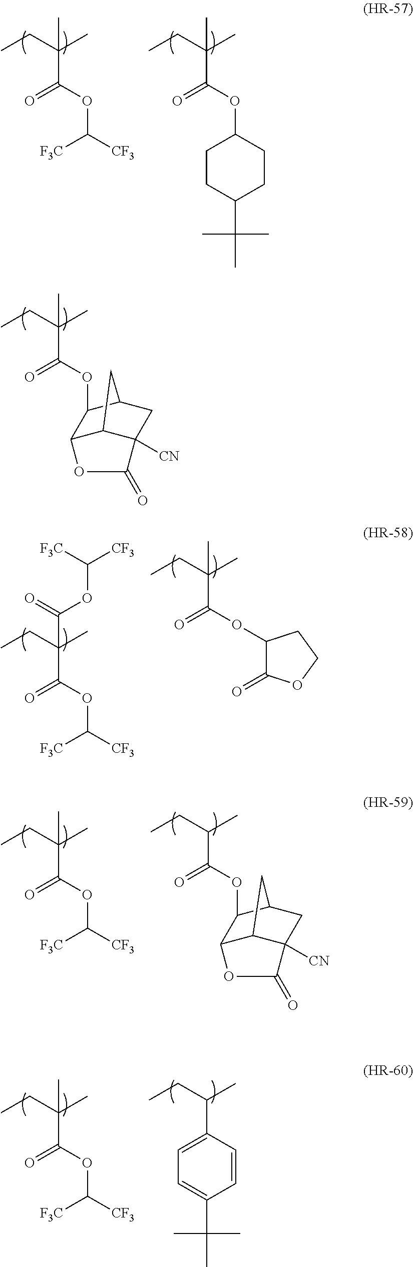

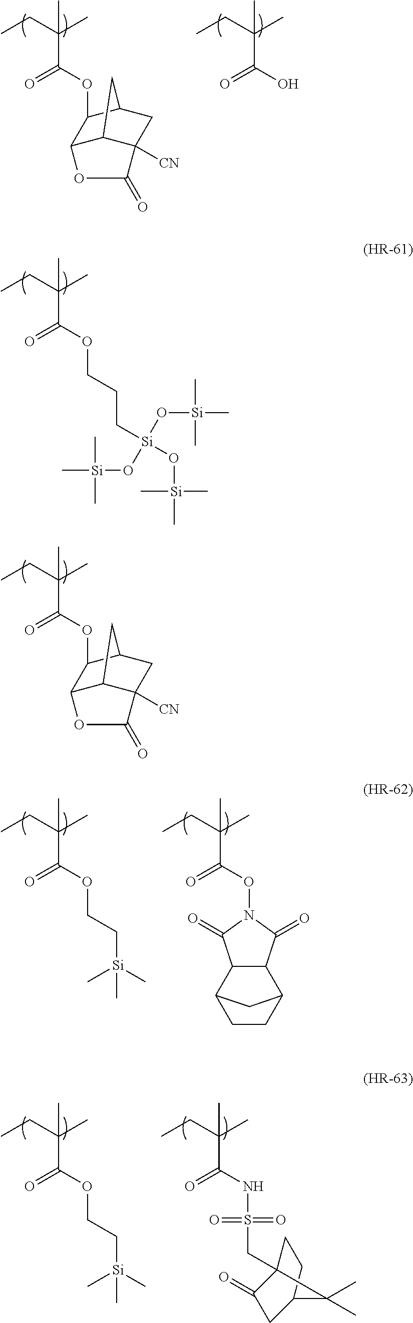

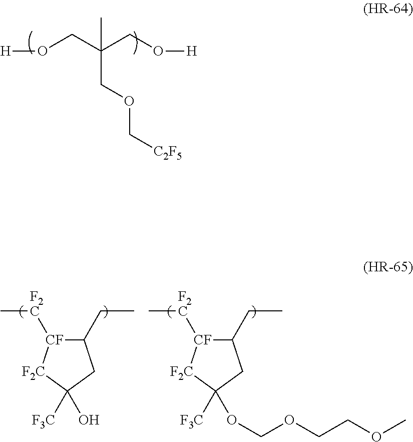

TABLE 1

| Resin | Composition | Mw | Mw/Mn |
|---|---|---|---|
| HR-1 | 50/50 | 4900 | 1.4 |
| HR-2 | 50/50 | 5100 | 1.6 |
| HR-3 | 50/50 | 4800 | 1.5 |
| HR-4 | 50/50 | 5300 | 1.6 |
| HR-5 | 50/50 | 4500 | 1.4 |
| HR-6 | 100 | 5500 | 1.6 |
| HR-7 | 50/50 | 5800 | 1.9 |
| HR-8 | 50/50 | 4200 | 1.3 |
| HR-9 | 50/50 | 5500 | 1.8 |
| HR-10 | 40/60 | 7500 | 1.6 |
| HR-11 | 70/30 | 6600 | 1.8 |
| HR-12 | 40/60 | 3900 | 1.3 |
| HR-13 | 50/50 | 9500 | 1.8 |
| HR-14 | 50/50 | 5300 | 1.6 |
| HR-15 | 100 | 6200 | 1.2 |
| HR-16 | 100 | 5600 | 1.6 |
| HR-17 | 100 | 4400 | 1.3 |
| HR-18 | 50/50 | 4300 | 1.3 |
| HR-19 | 50/50 | 6500 | 1.6 |
| HR-20 | 30/70 | 6500 | 1.5 |
| HR-21 | 50/50 | 6000 | 1.6 |
| HR-22 | 50/50 | 3000 | 1.2 |
| HR-23 | 50/50 | 5000 | 1.5 |
| HR-24 | 50/50 | 4500 | 1.4 |
| HR-25 | 30/70 | 5000 | 1.4 |
| HR-26 | 50/50 | 5500 | 1.6 |
| HR-27 | 50/50 | 3500 | 1.3 |
| HR-28 | 50/50 | 6200 | 1.4 |
| HR-29 | 50/50 | 6500 | 1.6 |
| HR-30 | 50/50 | 6500 | 1.6 |
| HR-31 | 50/50 | 4500 | 1.4 |
| HR-32 | 30/70 | 5000 | 1.6 |
| HR-33 | 30/30/40 | 6500 | 1.8 |
| HR-34 | 50/50 | 4000 | 1.3 |
| HR-35 | 50/50 | 6500 | 1.7 |
| HR-36 | 50/50 | 6000 | 1.5 |
| HR-37 | 50/50 | 5000 | 1.6 |
| HR-38 | 50/50 | 4000 | 1.4 |
| HR-39 | 20/80 | 6000 | 1.4 |
| HR-40 | 50/50 | 7000 | 1.4 |
| HR-41 | 50/50 | 6500 | 1.6 |
| HR-42 | 50/50 | 5200 | 1.6 |
| HR-43 | 50/50 | 6000 | 1.4 |
| HR-44 | 70/30 | 5500 | 1.6 |
| HR-45 | 50/20/30 | 4200 | 1.4 |
| HR-46 | 30/70 | 7500 | 1.6 |
| HR-47 | 40/58/2 | 4300 | 1.4 |
| HR-48 | 50/50 | 6800 | 1.6 |
| HR-49 | 100 | 6500 | 1.5 |
| HR-50 | 50/50 | 6600 | 1.6 |
| HR-51 | 30/20/50 | 6800 | 1.7 |
| HR-52 | 95/5 | 5900 | 1.6 |
| HR-53 | 40/30/30 | 4500 | 1.3 |
| HR-54 | 50/30/20 | 6500 | 1.8 |
| HR-55 | 30/40/30 | 7000 | 1.5 |
| HR-56 | 60/40 | 5500 | 1.7 |
| HR-57 | 40/40/20 | 4000 | 1.3 |
| HR-58 | 60/40 | 3800 | 1.4 |
| HR-59 | 80/20 | 7400 | 1.6 |
| HR-60 | 40/40/15/5 | 4800 | 1.5 |
| HR-61 | 60/40 | 5600 | 1.5 |
| HR-62 | 50/50 | 5900 | 2.1 |
| HR-63 | 80/20 | 7000 | 1.7 |
| HR-64 | 100 | 5500 | 1.8 |
| HR-65 | 50/50 | 9500 | 1.9 |

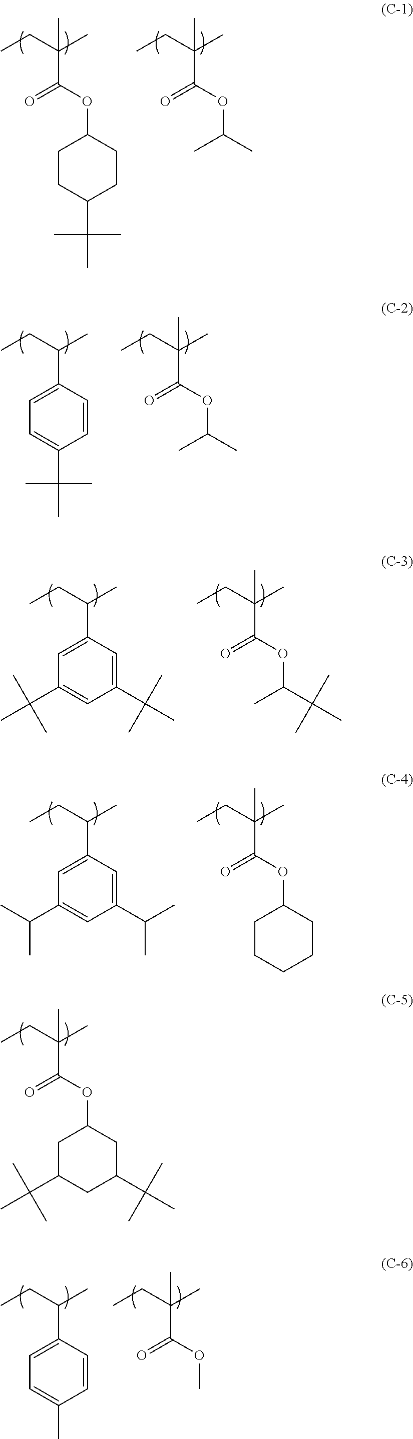

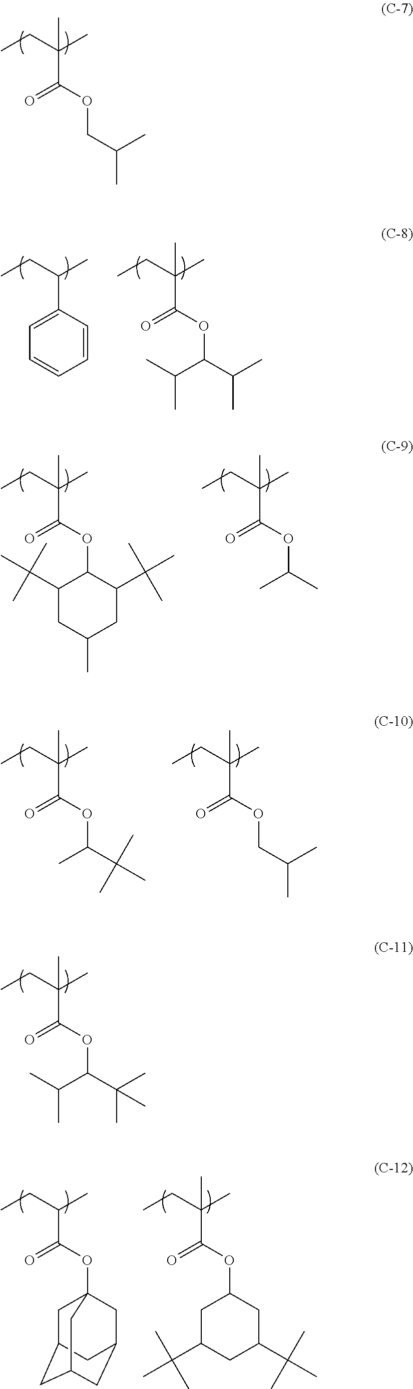

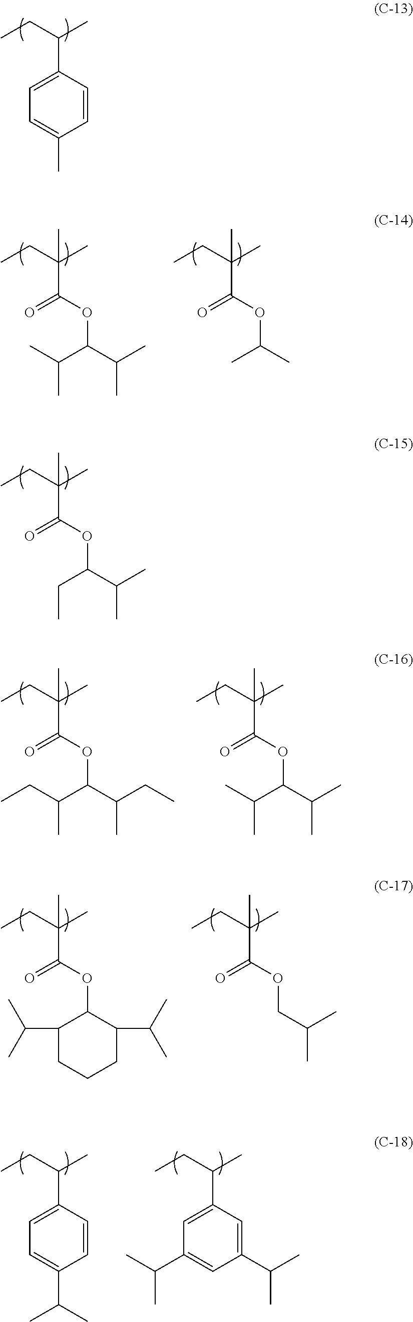

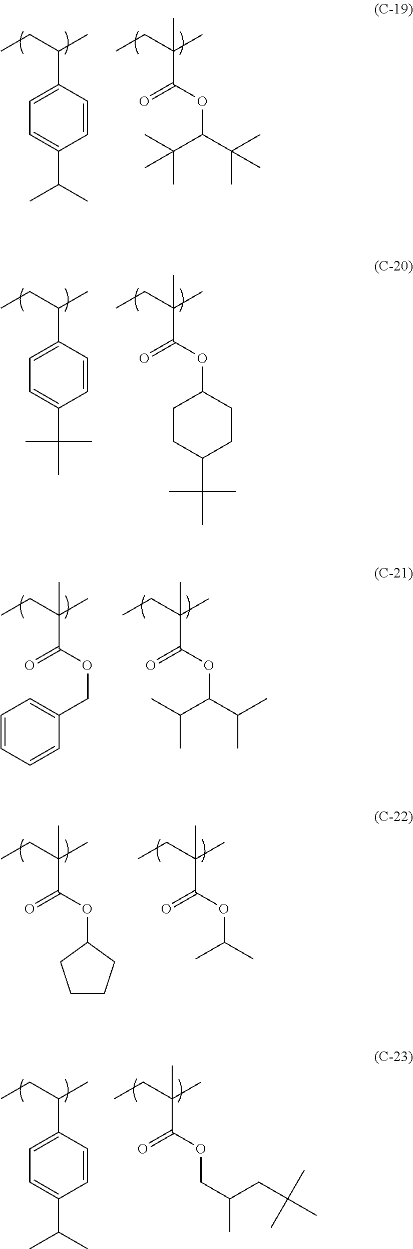

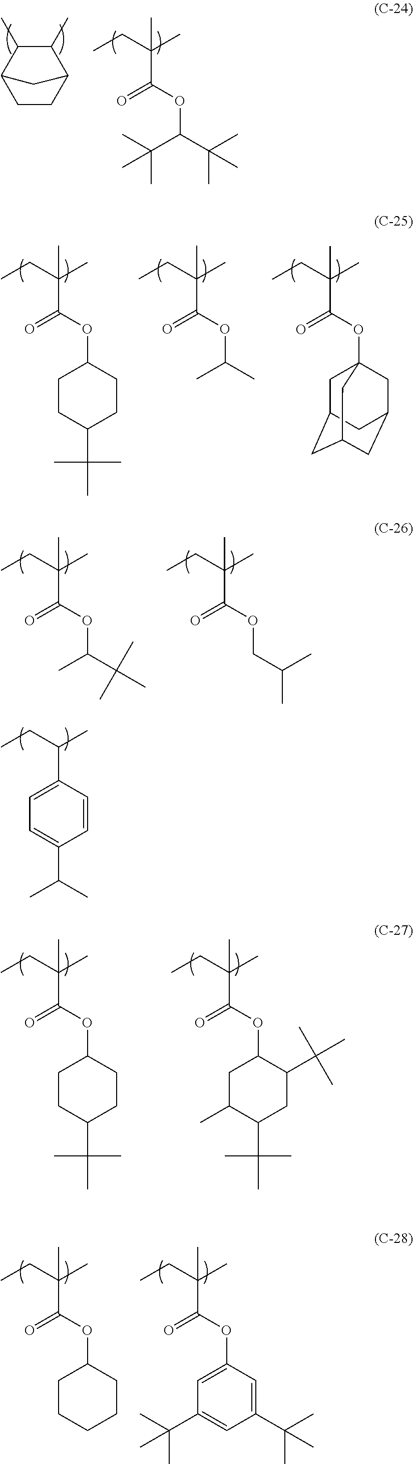

TABLE 2

| Resin | Composition | Mw | Mw/Mn |
|---|---|---|---|
| C-1 | 50/50 | 9600 | 1.74 |
| C-2 | 60/40 | 34500 | 1.43 |
| C-3 | 30/70 | 19300 | 1.69 |
| C-4 | 90/10 | 26400 | 1.41 |
| C-5 | 100 | 27600 | 1.87 |
| C-6 | 80/20 | 4400 | 1.96 |
| C-7 | 100 | 16300 | 1.83 |
| C-8 | 5/95 | 24500 | 1.79 |
| C-9 | 20/80 | 15400 | 1.68 |
| C-10 | 50/50 | 23800 | 1.46 |
| C-11 | 100 | 22400 | 1.57 |
| C-12 | 10/90 | 21600 | 1.52 |
| C-13 | 100 | 28400 | 1.58 |
| C-14 | 50/50 | 16700 | 1.82 |
| C-15 | 100 | 23400 | 1.73 |
| C-16 | 60/40 | 18600 | 1.44 |
| C-17 | 80/20 | 12300 | 1.78 |
| C-18 | 40/60 | 18400 | 1.58 |
| C-19 | 70/30 | 12400 | 1.49 |
| C-20 | 50/50 | 23500 | 1.94 |
| C-21 | 10/90 | 7600 | 1.75 |
| C-22 | 5/95 | 14100 | 1.39 |
| C-23 | 50/50 | 17900 | 1.61 |
| C-24 | 10/90 | 24600 | 1.72 |
| C-25 | 50/40/10 | 23500 | 1.65 |
| C-26 | 60/30/10 | 13100 | 1.51 |
| C-27 | 50/50 | 21200 | 1.84 |
| C-28 | 10/90 | 19500 | 1.66 |

[7] Solvent

An actinic-ray-sensitive or radiation-sensitive resin composition generally contains a solvent.

Examples of solvents which are able to be used when preparing an actinic-ray-sensitive or radiation-sensitive resin composition include organic solvents such as alkylene glycol monoalkyl ether carboxylate, alkylene glycol monoalkyl ether, alkyl lactate ester, alkoxypropionic acid alkyl, cyclic lactone (preferably 4 to 10 carbon atoms), a monoketone compound which may have a ring (preferably with 4 to 10 carbon atoms), alkylenecarbonate, alkoxy alkyl acetate, and alkyl pyruvate. Specific examples of the solvents include the solvents which are described in paragraphs 0441 to 0455 of US2008/0187860A.

In the present invention, a mixed solvent where a solvent which contains a hydroxyl group in the structure as an organic solvent and a solvent which does not contain a hydroxyl group are mixed may be used.

It is possible to appropriately select the compounds in the specific examples described above as the solvent which contains a hydroxyl group and the solvent which does not contain a hydroxyl group; however, alkylene glycol monoalkyl ether, alkyl lactate, and the like are preferable as the solvent which contains a hydroxyl group, and propylene glycol monomethyl ether (PGME, also called 1-methyoxy-2-propanol) and ethyl lactate are more preferable. In addition, alkylene glycol monoalkyl ether acetate, alkylalkoxypropionate, a monoketone compound which may contain a ring, cyclic lactone, alkyl acetate, and the like are preferable as the solvent which does not contain a hydroxyl group, propylene glycol monomethyl ether acetate (PGMEA, also called 1-methoxy-2-acetoxypropane), ethylethoxypropionate, 2-heptanone, γ-butyrolactone, cyclohexanone, and butyl acetate are particularly preferable among these, and propylene glycol monomethyl ether acetate, ethylethoxypropionate, and 2-heptanone are the most preferable.

The mixing ratio (mass) of the solvent which contains a hydroxyl group and the solvent which does not contain a hydroxyl group is 1/99 to 99/1, preferably 10/90 to 90/10, and more preferably 20/80 to 60/40. In terms of the coating uniformity, a mixed solvent which contains 50 mass % or more of the solvent which does not contain a hydroxyl group is particularly preferable.

The solvent preferably includes propylene glycol monomethyl ether acetate and is preferably a propylene glycol monomethyl ether acetate independent solvent or a mixed solvent of two or more types which contain propylene glycol monomethyl ether acetate.

[8] Other Additive Agents (G)

The actinic-ray-sensitive or radiation-sensitive resin composition in the present invention may or may not contain carbonic acid onium salt. Examples of the carbonic acid onium salt include the carbonic acid onium salt which is described in paragraphs 0605 and 0606 of US2008/0187860A.

It is possible to synthesize these carbonic acid onium salts by reacting sulfonium hydroxide, iodonium hydroxide, or ammonium hydroxide and carbonic acid with a silver oxide in an appropriate solvent.

In a case where the actinic-ray-sensitive or radiation-sensitive resin composition contains carbonic acid onium salt, the content is generally 0.1 mass % to 20 mass %, preferably 0.5 mass % to 10 mass %, and more preferably 1 mass % to 7 mass % with respect to the total solid content of the composition.

It is possible to further contain a compound (for example, a phenol compound with molecular weight of 1000 or less, or an alicyclic or aliphatic compound which has a carboxyl group), which promotes solubility with respect to an acid multiplication agent, a dye, a plasticizer, a photosensitizer, a light absorption agent, an alkali-soluble resin, a dissolution inhibitor, and a developing solution and the like in the actinic-ray-sensitive or radiation-sensitive resin composition of the present invention according to necessity.

It is possible for a person skilled in the art to easily synthesize the phenol compound with a molecular weight of 1000 or less with reference to a method disclosed in, for example, JP1992-122938A (JP-H4-122938A), JP1990-28531A (JP-H2-28531A), U.S. Pat. No. 4,916,210A, EP219294A, and the like.

Specific examples of an alicyclic or aliphatic compound which has a carboxyl group include a carbonic acid derivative which has a steroid structure such as cholic acid, deoxycholic acid, and lithocholic acid, an adamantane carbonic acid derivative, adamantane dicarbonic acid, cyclohexane carbonic acid, cyclohexane dicarbonic acid, and the like; however, the present invention is not limited thereto.

The actinic-ray-sensitive or radiation-sensitive resin composition in the present invention is preferably a resist film with film thickness of 80 nm or less from the point of view of improving resolution. It is possible to set the film thickness by setting the solid content concentration in the composition to an appropriate range to have a suitable viscosity and improving coating property and film-forming property.

The solid content concentration of the actinic-ray-sensitive or radiation-sensitive resin composition in the present invention is generally 1.0 mass % to 10 mass %, preferably 2.0 mass % to 5.7 mass %, and more preferably 2.0 mass % to 5.3 mass %. By setting the solid content concentration to these ranges, it is possible to uniformly coat the resist solution on a substrate and additionally, it is possible to form a resist pattern with excellent line width roughness (LWR). The reason is not clear; however, it is considered that, by setting the solid content concentration to 10 mass % or less and preferably 5.7 mass % or less, the aggregation of materials, particularly the photoacid generator, in the resist solution is suppressed and, as the result, it is possible to form a uniform resist film.

The solid content concentration is the mass percentage of the mass of other the resist components excluding the solvent with respect to the total mass of the actinic-ray-sensitive or radiation-sensitive resin composition.

The actinic-ray-sensitive or radiation-sensitive resin composition in the present invention is coated on a predetermined support body (substrate) to be used after dissolving the components described above in a predetermined organic solvent, preferably the mixed solvent, and filtering. The pore size of the filter which is used for the filtering is 0.1 μm or less, more preferably 0.05 μm or less, and even more preferably 0.03 μm or less and polytetrafluoroethylene, polyethylene, or nylon filters are preferable. In the filtering, for example, circulative filtering as in JP2002-62667A may be performed or filtering may be performed by connecting a plurality of types of filters in series or in parallel. In addition, a composition may be filtered a plurality of times. Furthermore, before or after filtering, a degassing process or the like may be performed with respect to the composition.

<Pattern Forming Method>

Next, description will be given of a pattern forming method according to the present invention.

The pattern forming method of the present invention includes at least exposing a resist film of the present invention and developing the exposed resist film.

In detail, (a) a step of forming a film (a resist film) which includes the actinic-ray-sensitive or radiation-sensitive resin composition of the present invention, (b) a step (an exposure step) of irradiating the film with actinic rays or radiation, and (c) a step of developing the film described above irradiated with actinic rays or radiation using a developing solution are at least included.

The exposure in the step (b) described above may be liquid immersion exposure.

The pattern forming method of the present invention preferably includes (d) a heating step (a step of heating after the exposure (PEB; Post Exposure Bake)) after (b) the exposure step.

The pattern forming method of the present invention may further include (e) a developing step using an alkali developing solution.

The pattern forming method of the present invention may include (b) the exposure step a plurality of times.

The pattern forming method of the present invention may include (d) the heating step a plurality of times.

The resist film of the present invention is formed of the actinic-ray-sensitive or radiation-sensitive resin composition of the present invention described above and more specifically, is preferably a film which is formed by coating the actinic-ray-sensitive or radiation-sensitive resin composition on a substrate. In the pattern forming method of the present invention, it is possible to perform a step of forming a film on a substrate using the actinic-ray-sensitive or radiation-sensitive resin composition, a step of exposing the film, and a developing step using methods which are generally known.

It is also preferable to include a preheating step (PB; Prebake) after film-forming and before the exposure step.

In addition, it is also preferable to include a post-exposure heating step after the exposure step and before the developing step.

It is preferable to perform both PB and PEB at a heating temperature of 70° C. to 130° C. and more preferably at 80° C. to 120° C.

The heating time is preferably 30 seconds to 300 seconds, more preferably 30 seconds to 180 seconds, and even more preferably 30 seconds to 90 seconds.

It is possible to perform the heating with means which is provided in a general exposure and developing machine and a hot plate or the like may be used.

Due to the baking, the reaction of an exposed section is promoted and the sensitivity or pattern profile is improved.

There is no limit on the wavelength of the light source which is used for the exposure apparatus in the present invention; however, examples thereof include infrared light, visible light, ultraviolet light, far ultraviolet light, extreme ultraviolet light, X-rays, electron beams, and the like, and far ultraviolet light with a wavelength of preferably 250 nm or less, more preferably 220 nm or less, and particularly preferably 1 nm to 200 nm, specifically, a KrF excimer laser (248 nm), an ArF excimer laser (193 nm), an F₂ excimer laser (157 nm), X-rays, EUV (13 nm), electron beams, and the like, and a KrF excimer laser, an ArF excimer laser, EUV, or electron beams are preferable, and an ArF excimer laser is more preferable.

In addition, it is possible to apply a liquid immersion exposure method in the exposure step of the present invention. It is possible to combine the liquid immersion exposure method with a super-resolution techniques such as a phase shift method and a modified lighting method.

In a case of performing liquid immersion exposure, a step of cleaning the surface of the film with a water-based chemical liquid may be carried out (1) after forming the film on a substrate and before an exposure step, and/or (2) after a step of carrying out exposure on a film via a liquid immersion liquid and before a step of heating the film.

The liquid immersion liquid is preferably a liquid which is transparent with respect to the exposure wavelength and where the temperature coefficient of the refractive index is as small as possible in order to keep deformation of an optical image which is projected on a film to a minimum; however, in particular, in a case where the exposure light source is an ArF excimer laser (wavelength; 193 nm), it is preferable to use water in terms of ease of availability and ease of handling in addition to the points of view described above.

In a case of using water, an additive agent (a liquid) which increases surface activity in addition to reducing the surface tension of the water may be added in a small ratio. The additive agent preferably does not dissolve a resist layer on a wafer and any influence with respect to an optical coating on a lower surface of a lens element of the exposure light source is negligible.

The additive agent is preferably an aliphatic alcohol which has substantially the same refractive index as water and specific examples thereof include methyl alcohol, ethyl alcohol, an isopropyl alcohol, and the like. By adding alcohol which has substantially the same refractive index as water, even when the alcohol components in water are evaporated and the content concentration changes, it is possible to obtain an advantage in that it is possible to make the refractive index change for the liquid as a whole extremely small.

On the other hand, since deformation of the optical image which is projected on the resist is caused in a case where a substance which is opaque with respect to 193 nm light or impurities where the refractive index is greatly different from water are mixed into a liquid immersion liquid, distilled water is preferable as water to be used. Furthermore, pure water where filtering is performed through an ion exchange filter and the like may also be used.

It is desirable that the electrical resistance of the water which is used as the liquid immersion liquid is 18.3 MΩcm or more, it is desirable that the TOC (organic concentration) is 20 ppb or less, and it is desirable that a degassing process is carried out.

In addition, by increasing the refractive index of the liquid immersion liquid, it is possible to increase the lithographic performance. From this point of view, an additive agent which increases the refractive index may be added to the water, or heavy water (D₂O) may be used instead of water.

A receding contact angle of the resist film which is formed using the actinic-ray-sensitive or radiation-sensitive resin composition in the present invention is 70° or more at a temperature of 23±3° C. and a humidity of 45±5% which is favorable in a case of exposure via the liquid immersion liquid, 75° or more is preferable, and 75° to 85° is more preferable.

When the receding contact angle is excessively small, favorable use is not possible in a case of exposure via a liquid immersion liquid and it is not possible to sufficiently exhibit the effect of reducing defects due to remaining water (water marks). In order to realize a favorable receding contact angle, it is preferable to include the hydrophobic resin (D) in the actinic-ray-sensitive or radiation-sensitive resin composition. Alternatively, the receding contact angle may be improved by forming a coating layer (a so-called “top coat”) with a hydrophobic resin composition on a resist film.

In the liquid immersion exposure step, since it is necessary for the liquid immersion liquid to move on a wafer following the movement of an exposure head scanning on the wafer at a high speed and forming exposure patterns, the contact angle of the liquid immersion liquid with respect to the resist film in a dynamic state is important and there is a demand for the resist to have a performance which follows the high speed scanning of the exposure head without liquid droplets remaining.

The substrate which forms the film in the present invention is not particularly limited, and it is possible to use a substrate which is generally used in a step of manufacturing a semiconductor such as IC such as an inorganic substrate such as silicon, SiO₂ or SiN or a coating based inorganic substrate such as SOG, a step of manufacturing a circuit substrate such as liquid crystal or a thermal head, in addition to a lithography step for other types of photofabrication. Furthermore, as necessary, an antireflection film may be formed between the resist film and the substrate. It is possible to appropriately use an organic or inorganic antireflection film which is known in the art as an antireflection film.

The developing solution which is used in the step of developing a resist film which is formed using the actinic-ray-sensitive or radiation-sensitive resin composition of the present invention is not particularly limited; however, it is possible to use, for example, an alkali developing solution or a developing solution which contains an organic solvent (also referred to below as an organic-based developing solution).

In a case where the pattern forming method of the present invention further includes a step of carrying out development using a developing solution which contains an alkali developing solution, the usable alkali developing solution is not particularly limited; however, in general, a solution of 2.38 mass % of tetramethyl ammonium hydroxide is desirable. In addition, use is also possible by adding an appropriate amount of alcohols and a surfactant to the alkali solution.

The alkali concentration of the alkali developing solution is generally 0.1 mass % to 20 mass %.

The pH of the alkali developing solution is generally 10.0 to 15.0.

Pure water is used as a rinsing liquid in a rinsing step which is performed after alkali development and use is also possible by adding an appropriate amount of the surfactant.

In addition, it is possible to perform a process which removes developing solution or rinsing liquid which is attached to the pattern using a supercritical fluid after the developing process or the rinsing process.

In a case where the pattern forming method of the present invention further includes a step of developing using a developing solution which contains an organic solvent, it is possible to use a polar solvent such as a ketone-based solvent, an ester-based solvent, an alcohol-based solvent, an amide-based solvent, and an ether-based solvent, or a hydrocarbon-based solvent as the developing solution (the organic-based developing solution).

Examples of the ketone-based solvent include 1-octanone, 2-octanone, 1-nonanone, 2-nonanone, acetone, 2-heptanone(methyl amyl ketone), 4-heptanone, 1-hexanone, 2-hexanone, diisobutyl ketone, cyclohexanone, methyl cyclohexanone, phenyl acetone, methyl ethyl ketone, methyl isobutyl ketone, acetyl acetone, acetonyl acetone, ionone, diacetonyl alcohol, acetyl carbinol, acetophenone, methyl naphthyl ketone, isophorone, propylene carbonate, and the like.

Examples of the ester-based solvent include methyl acetate, butyl acetate, ethyl acetate, isopropyl acetate, pentyl acetate, isopentyl acetate, amyl acetate, propylene glycol monomethyl ether acetate, ethylene glycol monoethyl ether acetate, diethylene glycol monobutyl ether acetate, diethylene glycol monoethyl ether acetate, ethyl-3-ethoxypropionate, 3-methoxybutyl acetate, 3-methyl-3-methoxybutyl acetate, methyl formate, ethyl formate, butyl formate, propyl formate, ethyl lactate, butyl lactate, propyl lactate, and the like.

Examples of the alcohol-based solvent include alcohols such as methyl alcohol, ethyl alcohol, n-propyl alcohol, isopropyl alcohol, n-butyl alcohol, sec-butyl alcohol, tert-butyl alcohol, isobutyl alcohol, n-hexyl alcohol, n-heptyl alcohol, n-octyl alcohol, and n-decanol, glycol-based solvents such as ethylene glycol, diethylene glycol, and triethylene glycol, glycol ether-based solvents such as ethylene glycol monomethyl ether, propylene glycol monomethyl ether, ethylene glycol monoethyl ether, propylene glycol monoethyl ether, diethylene glycol monomethyl ether, triethylene glycol monoethyl ether, and methoxy methyl butanol, and the like.

Examples of the ether-based solvent include dioxane, tetrahydrofuran, and the like other than the glycol ether-based solvents described above.

As the amide-based solvent, it is possible to use, for example, N-methyl-2-pyrrolidone, N,N-dimethyl acetamide, N,N-dimethyl formamide, hexamethylphosphoric triamide, 1,3-dimethyl-2-imidazolidinone, and the like.

Examples of the hydrocarbon-based solvent include aromatic hydrocarbon-based solvents such as toluene and xylene and aliphatic hydrocarbon-based solvents such as pentane, hexane, octane, and decane.

A plurality of the solvents described above may be mixed, and may be used after mixing with a solvent other than the solvents described above or water. However, in order to sufficiently exhibit the effects of the present invention, it is preferable that the water content of the developing solution as a whole is less than 10 mass %, and it is more preferable that water is not substantially contained.

That is, it is preferable that the usage amount of an organic solvent with respect to an organic-based developing solution is 90 mass % or more to 100 mass % or less with respect to the total amount of the developing solution, and 95 mass % or more to 100 mass % or less is preferable.

In particular, it is preferable that the organic-based developing solution is a developing solution which contains at least one type of organic solvent which is selected from a group formed of a ketone-based solvent, an ester-based solvent, an alcohol-based solvent, an amide-based solvent, and an ether-based solvent.

The vapor pressure of the organic-based developing solution is preferably 5 kPa or less, more preferably 3 kPa or less, and particularly preferably 2 kPa or less at 20° C. By setting the vapor pressure of an organic-based developing solution to 5 kPa or less, evaporation of the developing solution on a substrate or inside a developing cup is suppressed, temperature uniformity in a wafer surface is improved, and as a result, the uniformity of the dimensions in the wafer surface is improved.

It is possible to add an appropriate amount of a surfactant to an organic-based developing solution as necessary.

The surfactant is not particularly limited; however, for example, it is possible to use ionic or non-ionic fluorine-based and/or silicon-based surfactants and the like. Examples of the fluorine-based and/or silicon-based surfactant include the surfactants which are described in JP1987-36663A (JP-S62-36663A), JP1986-226746A (JP-S61-226746A), JP1986-226745A (JP-S61-226745A), JP1987-170950A (JP-S62-170950A), JP1988-34540A (JP-S63-34540A), JP1995-230165A (JP-H7-230165A), JP1996-62834A (JP-H8-62834A), JP1997-54432A (JP-H9-54432A), JP1997-5988A (JP-H9-5988A), U.S. Pat. No. 5,405,720A, U.S. Pat. No. 5,360,692A, U.S. Pat. No. 5,529,881A, U.S. Pat. No. 5,296,330A, U.S. Pat. No. 5,436,098A, U.S. Pat. No. 5,576,143A, U.S. Pat. No. 5,294,511A, and U.S. Pat. No. 5,824,451A, and a non-ionic surfactant is preferable. The non-ionic surfactant is not particularly limited; however, it is more preferable to use a fluorine-based surfactant or a silicon-based surfactant.

The usage amount of the surfactant is generally 0.001 mass % to 5 mass %, preferably 0.005 mass % to 2 mass %, and even more preferably 0.01 mass % to 0.5 mass % with respect to the total amount of the developing solution.

It is possible to apply, for example, a method in which a substrate is dipped in a tank which is filled with a developing solution for a certain period (a dipping method), a method of developing by raising a developing solution on a substrate surface using surface tension and resting for a certain period (a paddle method), a method for spraying a developing solution onto a substrate surface (a spraying method), a method which carries on discharging a developing solution onto a substrate which is rotated at a certain speed while scanning developing solution discharging nozzles at a certain speed (a dynamic dispensing method), and the like, as the developing method.

In a case where the various types of developing methods described above include a step of discharging a developing solution from developing nozzles of a developing apparatus onto a resist film, the discharging pressure of the developing solution which is discharged (flow speed in each unit area of the developing solution which is discharged) is preferably 2 mL/sec/mm² or less, more preferably 1.5 mL/sec/mm² or less, and even more preferably 1 mL/sec/mm² or less. There is no lower limit on the flow speed; however, when considering throughput, 0.2 mL/sec/mm² or more is preferable.

By setting the discharging pressure of the developing solution which is discharged to the ranges described above, it is possible to remarkably reduce pattern defects deriving from the resist residue after developing.

Details of the mechanism are not clear; however, it is considered that, by setting the discharging pressure to the ranges described above, the pressure which the developing solution applies to the resist film is small and the resist film or resist pattern is suppressed from being unnecessarily scraped or broken.

Here, the discharging pressure (mL/sec/mm²) of the developing solution is a value at a developing nozzle opening in the developing apparatus.

Examples of a method for adjusting the discharging pressure of the developing solution include a method for adjusting the discharging pressure by a pump and the like, a method for changing the pressure by adjusting the pressure in the supply from a pressure tank, and the like.

In addition, after a step of developing using a developing solution which contains an organic solvent, a step of stopping developing while carrying out substitution with another solvent may be carried out.

It is preferable to include a step of cleaning using a rinsing liquid after the step of developing using a developing solution which contains an organic solvent.

The rinsing liquid which is used for the rinsing step after the step of developing using a developing solution which contains an organic solvent is not particularly limited as long as the resist pattern is not dissolved and it is possible to use a solution which includes a general organic solvent. It is preferable to use a rinsing liquid which contains at least one type of an organic solvent which is selected from a group formed of a hydrocarbon-based solvent, a ketone-based solvent, an ester-based solvent, an alcohol-based solvent, an amide-based solvent, and an ether-based solvent as the rinsing liquid.

Specific examples of the hydrocarbon-based solvent, the ketone-based solvent, the ester-based solvent, the alcohol-based solvent, the amide-based solvent, and the ether-based solvent include the same solvents as the description for the developing solution which contains an organic solvent.

After the step of developing using a developing solution which contains an organic solvent, a step of cleaning (a rinsing step) using a rinsing liquid which contains at least one type of an organic solvent which is selected from a group formed of a ketone-base solvent, an ester-based solvent, an alcohol-based solvent, and an amide-based solvent is more preferably performed, a step of cleaning using a rinsing liquid which contains an alcohol-based solvent or an ester-based solvent is more preferably performed, a step of cleaning using a rinsing liquid which contains a monovalent alcohol is particularly preferably performed, and most preferably a step of cleaning using a rinsing liquid which contains a monovalent alcohol with 5 or more carbon atoms is performed.

Here, examples of the monovalent alcohol which is used in the rinsing step include linear, branched, or cyclic monovalent alcohols and specifically, it is possible to use 1-butanol, 2-butanol, 3-methyl-1-butanol, tert-butyl alcohol, 1-pentanol, 2-pentanol, 1-hexanol, 4-methyl-2-pentanol, 1-heptanol, 1-octanol, 2-hexanol, cyclopentanol, 2-heptanol, 2-octanol, 3-hexanol, 3-heptanol, 3-octanol, 4-octanol, and the like, and, as particularly preferable monovalent alcohols with 5 or more carbon atoms, it is possible to use 1-hexanol, 2-hexanol, 4-methyl-2-pentanol, 1-pentanol, 3-metyl-1-butanol, and the like.

A plurality of each of the components may be mixed or may be mixed with an organic solvent other than the solvents described above for use.

The water content in the rinsing liquid is preferably 10 mass % or less, more preferably 5 mass % or less, and particularly preferably 3 mass % or less. By setting the water content to 10 mass % or less, it is possible to obtain favorable developing characteristics.

The vapor pressure of the rinsing liquid which is used after the step of developing using a developing solution which contains an organic solvent is preferably 0.05 kPa or more to 5 kPa or less, more preferably 0.1 kPa or more to 5 kPa or less, and most preferably 0.12 kPa or more to 3 kPa or less at 20° C. By setting the vapor pressure of the rinsing liquid to 0.05 kPa or more to 5 kPa or less, the temperature uniformity in the wafer surface is improved, additionally, swelling which is caused by the permeation of the rinsing liquid is suppressed, and the uniformity of the dimensions in the wafer surface is improved.

Use is also possible by adding an appropriate amount of a surfactant to the rinsing liquid.

In the rinsing step, a cleaning process is carried out on the wafer on which developing is performed using a developing solution which contains an organic solvent, using a rinsing liquid which contains an organic solvent. A method of the cleaning processing is not particularly limited; however, for example, it is possible to apply a method which carries on discharging a rinsing liquid onto a substrate which is rotated at a certain speed (a rotary coating method), a method which dips a substrate in a tank which is filled with a rinsing liquid for a certain period (a dip method), a method which sprays a rinsing liquid onto a substrate surface (a spraying method), and the like, and it is preferable to perform the cleaning process using the rotary coating method among these methods, to rotate the substrate at rotation speed of 2000 rpm to 4000 rpm after cleaning, and to remove the rinsing liquid from the substrate. In addition, it is also preferable to include a heating step (Post Bake) after the rinsing step. Due to baking, the developing solution and the rinsing liquid which remain between the patterns and in the patterns are removed. The heating step after the rinsing step is generally performed at 40° C. to 160° C., preferably 70° C. to 95° C., and generally for 10 seconds to 3 minutes, preferably 30 seconds to 90 seconds.

In addition, the present invention also relates to a producing method of an electronic device which includes the pattern forming method of the present invention described above and to an electronic device which is manufactured by the producing method.

The electronic device of the present invention is suitable for mounting on electrical and electronic equipment (household electrical appliances, OA and media-related apparatuses, optical equipment, telecommunication equipment, and the like).

### EXAMPLES

Description will be given below of the present invention using the examples; however, the present invention is not limited thereto.

### Synthesis Example 1

### Synthesis of PAG-1

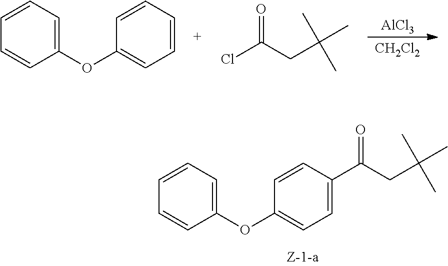

A mixed liquid was obtained by dissolving 6.8 g of phenylether in 30 mL of dichloromethane, cooling to 0° C., and then adding 5.8 g of ammonium chloride. 5.4 g of tert-butylacetyl chloride was dripped into the obtained mixed liquid at 0° C. and the reaction mixture was stirred at 0° C. for 2 hours. The reaction mixture was poured into a mixed solution of 60 mL of hexane/ethyl acetate (volume ratio 3/1) and 60 mL of ice water. After stirring for 10 minutes, the reactant was extracted from aqueous phase three times with 20 mL of hexane/ethyl acetate (volume ratio 3/1). After mixing the obtained organic phases and washing with 1N hydrochloric acid, water, saturated sodium bicarbonate solution, and brine, 10.6 g of a compound (Z-1-a) was obtained by distilling off the solvent (yield>99%).

¹H-NMR (CDCl₃, 300 MHz): d1.06 (s, 9H), 2.82 (s, 2H), 6.99 (d, 2H), 7.07 (d, 2H), 7.15 (t, 1H), 7.39 (t, 2H), and 7.93 (d, 2H).

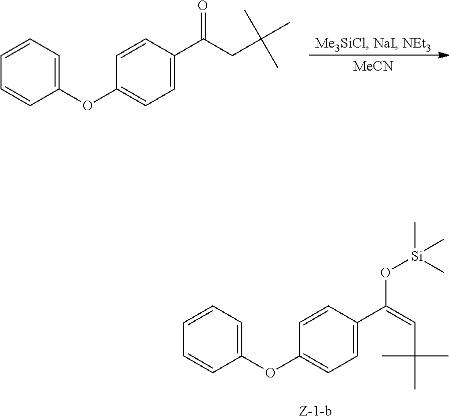

9.4 g of the compound (Z-1-a) was dissolved in 25 mL of acetonitrile and 7.9 g of sodium iodide and 8.9 g of triethylamine were added thereto. After adding 5.7 g of chlorotrimethylsilane dropwise to the obtained solution, the reaction mixture was stirred at 50° C. for 2 hours. After cooling to room temperature, the reaction mixture was poured into a mixed solution of 90 mL of hexane/ethyl acetate (volume ratio 3/1) and 90 mL of saturated sodium bicarbonate solution and stirred for 10 minutes. After extracting the reactant from the aqueous phase three times with 20 mL of hexane/ethyl acetate (volume ratio 3/1), the obtained organic phases were combined and washing with saturated sodium bicarbonate solution, water, and brine, and then 11.9 g of a compound (Z-1-b) was obtained by distilling off the solvent (yield>99%).

¹H-NMR (CDCl₃, 300 MHz): d0.00 (s, 9H), 1.10 (s, 9H), 4.71 (s, 9H), 6.83 (d, 2H), 6.92 (d, 2H), 7.01 (t, 1H), and 7.23 to 7.27 (m, 4H).

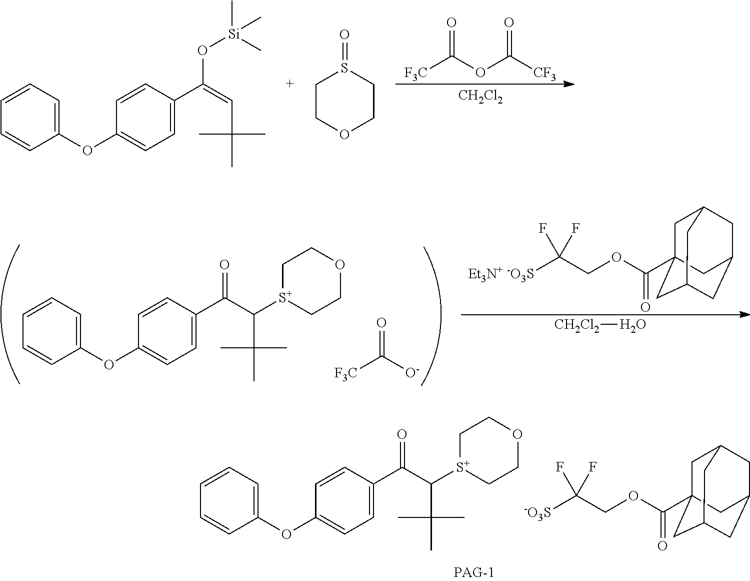

A mixed liquid was obtained by dissolving 11.9 g of the compound (Z-1-b) and 6.3 g of 1,4-thioxane-4-oxide in 68 mL of dichloromethane and cooling to −40° C. A dichloromethane solution (7.5 mL) of trifluoroacetic anhydride (11.0 g) was added dropwise while maintaining the mixed liquid at −35° C. or less, and the reaction mixture was stirred at −35° C. for 3 hours. After increasing the temperature to 0° C., 10 mL of water was added dropwise at 10° C. or less and subsequently 135 mL of saturated sodium bicarbonate aqueous solution was added dropwise at 10° C. or less. After increasing the temperature of the water solution after the reaction to room temperature, the water solution was stirred for 15 minutes, 15.6 g of triethylammonium 2-(adamantane-1-carbonyloxy)-1,1-difluoro-ethanesulfonate was added thereto and stirred for 30 minutes. After extracting the reactant from the aqueous phase with 60 mL of dichloromethane, the obtained organic phases were mixed and cleaned twice using 80 mL of 10 mass % potassium carbonate water and 5 times using 80 mL of water. After distilling off the solvent, 17.5 g of PAG-1 was obtained by carrying out recrystallization from diisopropylether (yield 72%).

¹H-NMR (CDCl₃, 300 MHz): d1.24 (s, 9H), 1.71 (d, 6H), 1.92 (d, 6H), 2.01 (brs, 2H), 3.32 (d, 1H), 3.68 (td, 1H), 3.83 to 3.97 (m, 4H), 4.25 (dt, 1H), 4.34 (dt, 1H), 4.76 (t, 2H), 6.09 (s, 1H), 6.07 (d, 2H), 7.07 (d, 2H), 7.09 (d, 2H), 7.24 (t, 1H), 7.42 (t, 2H), and 8.26 (d, 2H).

### Synthesis Example 2

### Synthesis of PAG-2 to PAG-7 and C-1 to C-6

PAG-2 to PAG-7 were synthesized in the same manner as Synthesis Example 1. In addition, C-1 to C-6 were synthesized by a method which is known in the art.

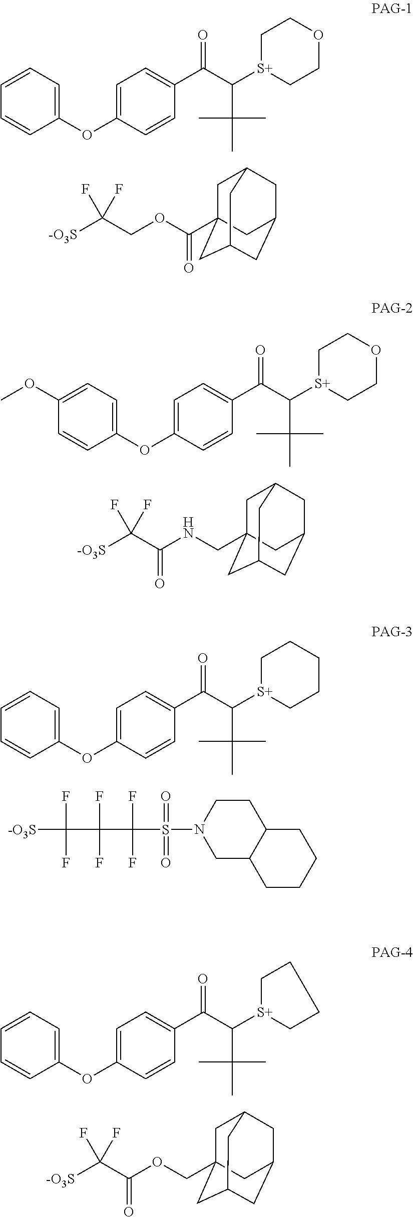

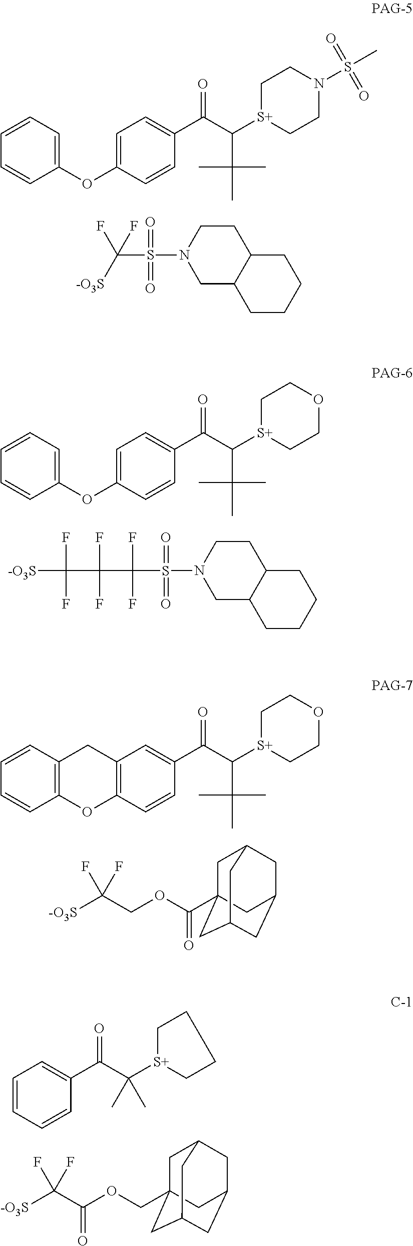

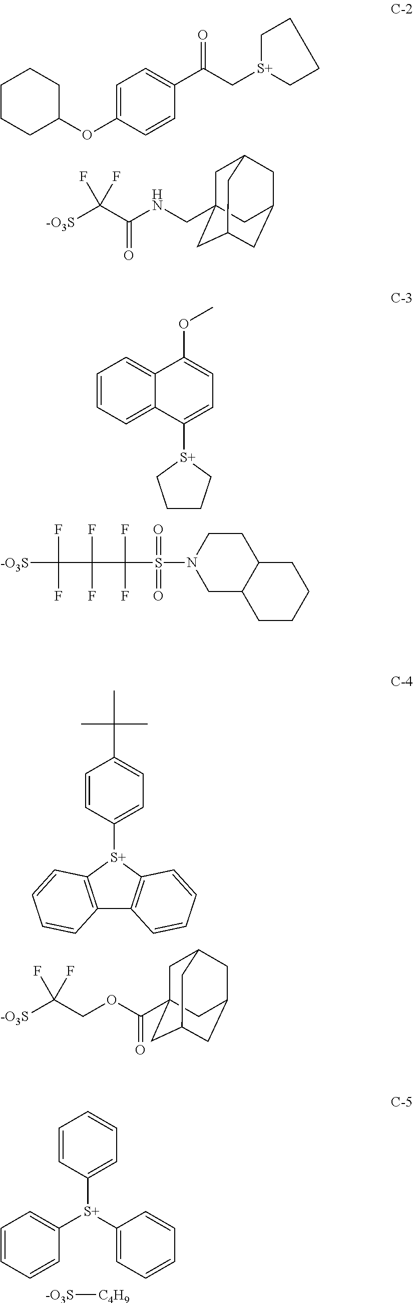

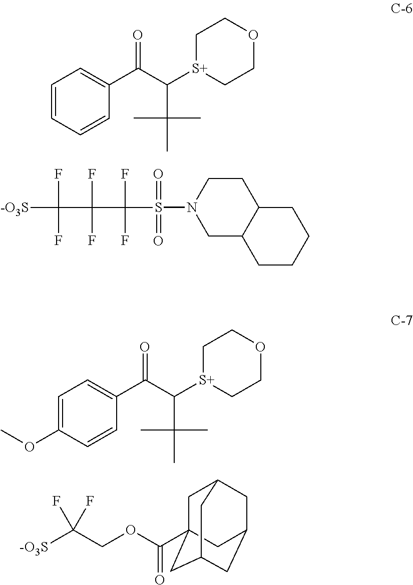

The relative molar absorbance coefficient (εᵣ), relative quantum efficiency (φᵣ), and relative acid generation efficiency (εᵣ×φᵣ) of PAG-1 to 7 and C-1 to 7 are shown below.

TABLE 3

| | Relative Molar | | Relative Acid |
|---|---|---|---|
| | Absorbance | Relative Quantum | Generation |
| | Coefficient (εᵣ) | Efficiency (φᵣ) | Efficiency (εᵣ × φᵣ) |
| PAG-1 | 0.55 | 1.27 | 0.698 |
| PAG-2 | 0.44 | 1.4 | 0.616 |
| PAG-3 | 0.6 | 1.25 | 0.75 |
| PAG-4 | 0.53 | 1.22 | 0.647 |
| PAG-5 | 0.6 | 1.25 | 0.75 |
| PAG-6 | 0.55 | 1.27 | 0.698 |
| PAG-7 | 0.59 | 1.30 | 0.767 |
| C-1 | 0.25 | 1.26 | 0.315 |
| C-2 | 0.27 | 0.8 | 0.216 |
| C-3 | 0.4 | 1.2 | 0.48 |
| C-4 | 0.65 | 0.21 | 0.1365 |
| C-5 | 1 | 1 | 1 |
| C-6 | 0.38 | 0.71 | 0.2698 |
| C-7 | 0.29 | 1.40 | 0.406 |

### Synthesis Example 3

### Synthesis of Polymer (1)

102.3 parts by mass of cyclohexanone were heated to 80° C. under a nitrogen gas stream. While stirring the liquid, a mixed solution of 22.2 parts by mass of a monomer which is represented by structural formula M-1 below, 22.8 parts by mass of a monomer which is represented by structural formula M-2 below, 6.6 parts by mass of a monomer which is represented by structural formula M-3 below, 189.9 parts by mass of cyclohexanone, and 2.40 parts by mass of 2,2′-azobisiso butyric acid dimethyl [V-601, manufactured by Wako Pure Chemical Industries, Ltd.] were added dropwise over 5 hours. After finishing the dropwise addition, the solution was further stirred for 2 hours at 80° C. After leaving the reaction liquid to cool, 41.1 parts by mass of Polymer (1) which is used in the present invention were obtained by re-precipitating and filtering with a large quantity of hexane/ethyl acetate (mass ratio 9:1) and vacuum drying the obtained solid matter.

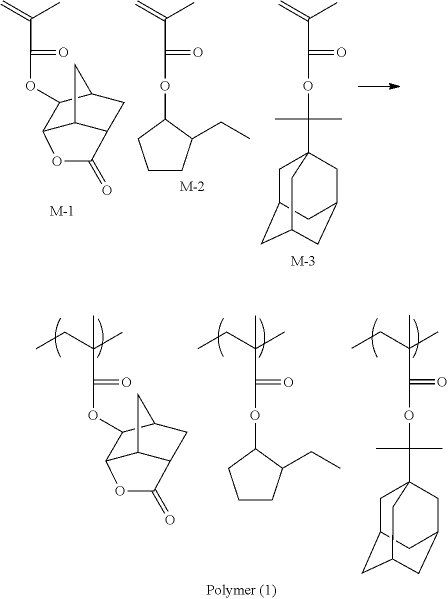

The weight average molecular weight (Mw: polystyrene conversion) Mw which was calculated from the GPC (carrier: tetrahydrofuran (THF)) of the obtained Polymer (1) was 9500 and the dispersity was Mw/Mn=1.60. The composition ratio (mol %) which was measured by ¹³C-NMR was M-1/M-2/M-3=40/50/10.

Polymers (2) to (9) were synthesized by performing the same operation as for Synthesis Example 3. The synthesized polymer structures, the composition ratio of each repeating unit (molar ratio; corresponding in order from left), the weight average molecular weight (Mw), and the dispersity (Mw/Mn) will be shown below.

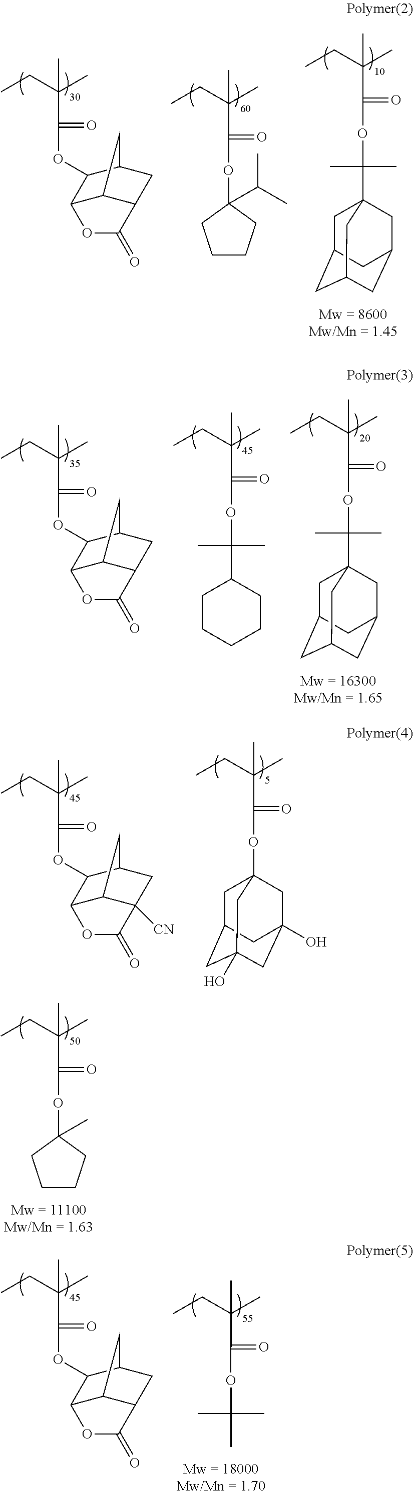

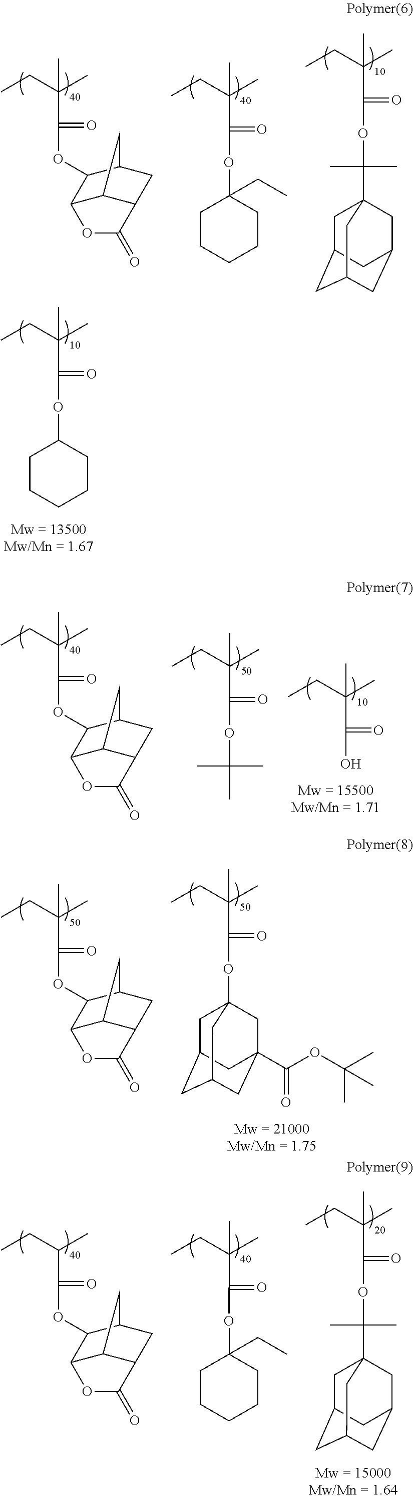

<Calculation of Relative Light Absorbance (εᵣ)>

Firstly, a molar absorbance coefficient (ε) was calculated for each of a target acid generating agent and triphenyl sulfonium nonaphlate. By using a cell with 1 cm corners to measure the UV spectrum of a measurement solution where a compound is dissolved in acetonitrile, the molar absorbance coefficient (ε) was calculated according to the Lambert-Beer formula from light absorbance (A) and measurement solvent density (C) with respect to light with a wavelength of 193 nm. The relative light absorbance εᵣ of a target acid generating agent is a value which is standardized when the light absorbance coefficient of triphenyl sulfonium nonaphlate is set as 1.

εᵣ=ε_{z}/ε_{TPS}

εᵣ: relative light absorbance of a target acid generating agent

ε_{z}: molar absorbance coefficient of a target acid generating agent

ε_{TPS}: molar absorbance coefficient of triphenyl sulfonium nonaphlate

<Calculation of Sensitivity Eᵣ and E_{TPS}>

A resist solution with solid content concentration of 3.5 mass % was obtained by dissolving 10 g of Polymer (1) which was used in Example 1, 0.3 g of a basic compound DIA, and 2.0 g of triphenyl sulfonium nonaphlate in a solvent (PGMEA). A resist composition was prepared by filtering the resultant using a polyethylene filter with a pore size of 0.03 μm. ARC29SR (manufactured by Nissan Chemical Industries, Ltd.) for an organic antireflection film was coated on a silicon wafer, baking was performed at 205° C. for 60 seconds, and an antireflection film with a film thickness of 100 nm was formed. A resist composition was coated thereon, baking (PB: Prebake) was performed at 100° C. for 60 seconds, and a resist film with film thickness of 100 nm was formed. The entire surface of the obtained wafer was exposed using an ArF excimer laser scanner (manufactured by ASML; PAS5500/1100). After that, heating (PEB: Post Exposure Bake) was carried out for 60 seconds at 100° C. Subsequently, developing was carried out by paddling using an organic-based developing solution (butyl acetate) for 30 seconds and rinsing was carried out by paddling using a rinsing liquid (methylisobutylcarbinol (MIBC)) for 30 seconds while shaking off the developing solution. Subsequently, after rotating the wafer at a rotation speed of 4000 rpm for 30 seconds, baking was performed for 60 seconds at 90° C. After that, the film thickness after baking was measured.

By increasing the exposure amount from 1 mJ/cm² by 0.3 mJ/cm² increments, the exposure amount when the film thickness after baking exceeded 10 nm was defined as the sensitivity E_{TPS} of triphenyl sulfonium nonaphlate. The acid generating agent was changed from triphenyl sulfonium nonaphlate to a target acid generating agent and the sensitivity Eᵣ of the target acid generating agent was measured with the same steps.

<Calculation of Relative Quantum Efficiency (φᵣ)>

The relative quantum efficiency (φᵣ) is defined as φᵣ=(φ_{TPS}×ε_{TPS}×E_{TPS})/εᵣ×Eᵣ when the relative light absorbance of the target acid generating agent is εᵣ, the relative quantum efficiency is εᵣ, the sensitivity is Eᵣ, the mol absorbance coefficient of triphenyl sulfonium nonaphlate is ε_{TPS}, the relative quantum efficiency is φ_{TPS}, and the sensitivity is E_{TPS}. Here, ε_{TPS} and φ_{TPS} are 1 and E_{TPS} and Eᵣ are obtained by the measuring method described above. In the measurement of Eᵣ, the types and amounts of resins, basic compounds, and solvents were set to the same measuring conditions as for E_{TPS}. The amount of the acid generating agent was set to be the same as the measuring conditions for E_{TPS} with the amount of the substance (molar quantity) as a reference. The relative quantum efficiency φᵣ of the target acid generating agent was calculated by substituting the measured values of εᵣ, Eᵣ, and E_{TPS} in the formula described above.

<Basic Compound>

DIA: 2,6-diisopropylaniline

TEA: triethanolamine

DBA: N,N-diputylaniline

PBI: 2-phenylbenzimidazole

PEA: N-phenyldiethanolamine

In addition, compounds (N-1) to (N-5) below were used as the basic compound.

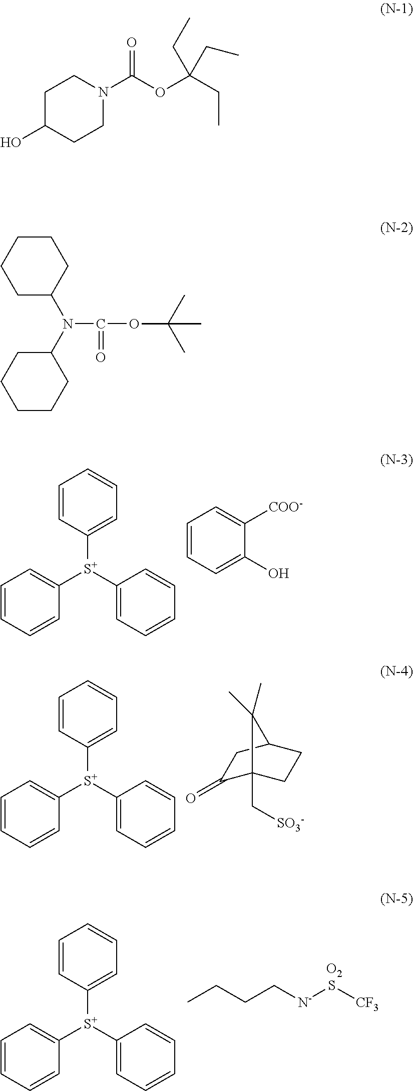

<Hydrophobic Resin (D)>

A hydrophobic resin (D) was used by appropriately selecting from the resins (HR-1) to (HR-65) and (C-1) to (C-28) described above.

<Surfactant>

W-1: Megaface F176 (manufactured by DIC Inc.) (fluorine-based)

W-2: Megaface R08 (manufactured by DIC Inc.) (fluorine-based and silicon-based)

W-3: PF6320 (manufactured by OMNOVA Solutions Inc.) (fluorine-based)

W-4: Troyzol S-366 (manufactured by Troy Chemical Industries, Inc.)

<Solvent>

A1: propylene glycol monomethyl ether acetate (PGMEA)

A2: cyclohexanone

A3: γ-butyrolactone

B1: propylene glycol monomethyl ether (PGME)

B2: ethyl lactate

<Developing Solution>

SG-1: butyl acetate

SG-2: methyl amyl ketone

SG-3: ethyl-3-ethoxypropionate

SG-4: pentyl acetate

SG-5: isopentyl acetate

SG-6: propylene glycol monomethyl ether acetate (PGMEA)

SG-7: cyclohexanone

<Rinsing Liquid>

SR-1: 4-methyl-2-pentanol

SR-2: 1-hexanol

SR-3: butyl acetate

SR-4: methyl amyl ketone

SR-5: ethyl-3-ethoxypropionate

### Examples 1 to 13 and Comparative Examples 1 to 11

An actinic-ray-sensitive or radiation-sensitive resin composition (a resist composition) was prepared by dissolving 3.5 mass % of components shown in Table 4 below in a solvent shown in the same table as solid matter and filtering each component using a polyethylene filter with a pore size of 0.03 ARC29SR (manufactured by Nissan Chemical Industries, Ltd.) for an organic antireflection film was coated on a silicon wafer, baking was performed for 60 seconds at 205° C., and an antireflection film with a film thickness of 100 nm was formed. An actinic-ray-sensitive or radiation-sensitive resin composition was coated thereon, baking (PB: Prebake) was performed for 60 seconds at 100° C., and a resist film with film thickness of 80 nm was formed. Patterning exposure was performed on the obtained wafer using an ArF excimer laser liquid immersion scanner (manufactured by ASML; XT1700i, NA1.20, C-Quad, outer sigma 0.981, inner sigma 0.895, and XY inclination) via an exposure mask (line/space=binary mask 44 nm/44 nm). Ultra-pure water was used as the liquid immersion liquid. After that, heating (PEB: Post Exposure Bake) was carried out at the temperatures shown in Table 4 for 60 seconds. Subsequently, developing was carried out by paddling the developing solution shown in Table 4 for 30 seconds and rinsing was carried out by paddling using the rinsing liquid shown in Table 4 for 30 seconds while shaking off the developing solution. Subsequently, after rotating the wafer at a rotation speed of 4000 rpm for 30 seconds, baking was performed for 60 seconds at 90° C. A resist pattern of a 1:1 line and space pattern with a line width of 44 nm was obtained in this manner.

<Preservation Stability (Sensitivity)>

In the obtained resist film, the exposure amount (mJ/cm²) when the resist pattern of the 1:1 line and space pattern with a line width of 44 nm was formed was set as an optimal exposure amount. The smaller the value is, the higher the sensitivity is, which is preferable. Changes in sensitivity were evaluated using the ratio of an optimal exposure amount S1 in a case of using the resist solution directly after preparation and a suitable exposure amount S2 of a resist solution left at 4° C. for 1 week after preparation (S1/S2). When the value of S1/S2 is close to 1, the change in sensitivity is small, which is preferable.

<Preservation Stability (Particles)>

With regard to the prepared resist solution, the number of particles (a particle initial value) in the solution directly after preparation and the number of particles (the number of particles after the passing of time) in the solution after being left at 4° C. for 3 months were counted using a particle counter manufactured by Rion Co., Ltd. and the number of increased particles calculated by (the number of particles after passing of time)−(particle initial value) was calculated. Here, particles with a particle diameter of 0.25 μm or more included in 1 mL of a solution were counted. A case where the number of increased particles is equal to 0.2 per ml or less is set as A, a case of more than 0.2 per ml to 1 per ml or less is set as B, a case of more than 1 per ml to 5 per ml or less is set as C, and a case of more than 5 per ml is set as D.

<Development Defects>

After leaving the prepared resist solution at 4° C. for 3 months and forming a resist film by the method described above, a 1:1 line and space pattern with a line width of 44 nm was formed with the same method described above and measurement was carried out using a defect inspecting apparatus KLA2360 manufactured by KLA-Tencor Corporation in a random mode by setting the pixel size of the defect inspecting apparatus to 0.16 m and additionally setting a threshold to 20. Development defects extracted from the difference which is generated by the overlapping of a comparison image in pixel unit were detected, and the number of development defects in each unit area was calculated. A small value indicates a favorable performance.

<Pattern Shape>

A sectional shape of the line pattern of the 1:1 line and space pattern with a line width of 44 nm, which was obtained by the minimum exposure amount (Eopt) for reproducing the line pattern of the 1:1 line and space pattern with a line width of 44 nm of a mask, was observed using a scanning electron microscope. The pattern shape was evaluated using a ratio (a/b) of a length a (nm) of the upper side of the pattern and a length b (nm) of the lower side of the pattern.

A case where a/b is 1.0 or more to less than 1.1 is set as A, a case of 1.1 or more to less than 1.3 is set as B, and a case of 1.3 or more is set as C. a/b is preferably close to 1 since the pattern shape is close to a rectangle.

<Line Width Roughness (LWR)>

In the measurement of the line and space resist pattern of the 1:1 line and space pattern with a line width of 44 nm which was resolved using the exposure amount with the sensitivity (Eopt) described above, when observing from the upper part of the pattern using a length measurement scanning electron microscope (SEM (Hitachi Ltd. S-8840)), observation of the line width was carried out at arbitrary points and the measurement variations thereof were evaluated at 3σ. A small value indicates a favorable performance.

<Pattern Collapse>

When the exposure amount for reproducing the mask pattern of the 1:1 line and space pattern with a line width of 44 nm is set as an optimal exposure amount and the exposure amount was further reduced from the optimal exposure amount, defining was carried out with a space width for developing without the pattern collapsing. A higher value represents that a finer pattern is resolved without collapsing and indicates that it is difficult for pattern collapse to be generated.

The evaluation results are shown in Table 5 below.

TABLE 4

| | | | Basic | Hydro- | | | | | PEB |
|---|---|---|---|---|---|---|---|---|---|
| | Acid | | Compound | phobic | | Surfac- | Developing | Rinsing | Temper- |
| | Generating | Resin (B) | or Compound | Resin (D) | Solvent | tant | Solution | Solution | ature |
| | Agent (g) | (10 g) | (C) (mg) | (35 mg) | (Mass Ratio) | (10 mg) | (Mass Ratio) | (Mass Ratio) | (° C.) |
| Example 1 | PAG-1(1.7) | Polymer(1) | DIA(0.29) | C-17 | A1 | W-1 | SG-1 | SR-1 | 100 |
| Example 2 | PAG-2(1.8) | Polymer(2) | PBI(0.31) | HR-12 | A1/B1(80/20) | W-1 | SG-1/SG-7 | SR-1 | 110 |
| | | | | | | | (95/5) | | |
| Example 3 | PAG-3(2.0) | Polymer(3) | DBA(0.41) | C-2 | A1/B1(60/40) | W-1 | SG-1 | SR-1/SR-4 | 105 |
| | | | | | | | | (90/10) | |
| Example 4 | PAG-4(1.7) | Polymer(4) | N-1(0.30) | C-3 | A1/B2(95/5) | W-2 | SG-1/SG-4 | SR-1 | 120 |
| | | | | | | | (50/50) | | |
| Example 5 | PAG-5(2.0) | Polymer(5) | TEA(0.31) | C-12 | A1/B3(95/5) | W-1 | SG-1 | SR-1 | 130 |
| Example 6 | PAG-6(2.0) | Polymer(6) | N-2(0.31) | C-18 | B2 | W-4 | SG-1 | SR-1 | 90 |
| Example 7 | PAG-7(1.8) | Polymer(3) | DBA(0.41) | C-2 | A1/B1(60/40) | W-1 | SG-1 | SR-1/SR-4 | 105 |
| | | | | | | | | (90/10) | |
| Example 8 | PAG-1/PAG-2 | Polymer(7) | N-1/PBI | HR-32 | A1/B1/A2 | W-1 | SG-1/SG-6 | SR-1/SR-3 | 90 |
| | (1.0/0.8) | | (0.15/0.1) | | (50/45/5) | | (95/5) | (90/10) | |
| Example 9 | PAG-1/C-5 | Polymer(8) | N-4(0.32) | HR-38 | A1/B1/A3 | W-3 | SG-2 | SR-1 | 100 |
| | (1.0/0.9) | | | | (50/45/5) | | | | |
| Example 10 | PAG-2/C-7 | Polymer(9) | N-5(0.29) | C-8 | A1/B1/B2 | W-2 | SG-1/SG-3(90/10) | SR-1 | 110 |
| | (1.2/0.7) | | | | (50/45/5) | | | | |
| Example 11 | PAG-1(1.7) | Polymer(1)/ | N-2(0.31) | HR-17 | A1 | W-1 | SG-1 | SR-1/SR-2 | 105 |
| | | Polymer(2) | | | | | | (90/10) | |
| | | (5 g/5 g) | | | | | | | |
| Example 12 | PAG-2(1.8) | Polymer(3)/ | N-3(0.41) | HR-19 | A1/B1(80/20) | W-4 | SG-1/SG-5 | SR-5 | 120 |
| | | Polymer(7) | | | | | (90/10) | | |
| | | (2 g/8 g) | | | | | | | |
| Example 13 | PAG-3(2.0) | Polymer(4)/ | N-1(0.30) | HR-56 | A1/B1(60/40) | W-1 | SG-7 | SR-1 | 130 |
| | | Polymer(5) | | | | | | | |
| | | (4 g/6 g) | | | | | | | |
| Comparative | C-1(1.8) | Polymer(7) | N-1(0.28) | C-17 | A1 | W-1 | SG-1 | SR-1 | 100 |
| Example 1 | | | | | | | | | |
| Comparative | C-2(1.9) | Polymer(8) | PBI(0.30) | HR-12 | A1/B2(90/10) | W-1 | SG-1 | SR-1 | 100 |
| Example 2 | | | | | | | | | |
| Comparative | C-3(2.0) | Polymer(1) | PEA(0.40) | C-2 | A1/B2(80/20) | W-1 | SG-1 | SR-1 | 100 |
| Example 3 | | | | | | | | | |
| Comparative | C-4(1.8) | Polymer(2) | DIA(0.31) | HR-38 | A1/B2(90/10) | W-1 | SG-1 | SR-1 | 100 |
| Example 4 | | | | | | | | | |
| Comparative | C-5(2.1) | Polymer(9) | PEA(0.3) | C-8 | A1 | W-1 | SG-1 | SR-1 | 100 |
| Example 5 | | | | | | | | | |
| Comparative | C-6(2.0) | Polymer(6) | PEA(0.40) | C-18 | A1/B2(80/20) | W-1 | SG-1 | SR-1 | 100 |
| Example 6 | | | | | | | | | |
| Comparative | C-1(3.8) | Polymer(4) | N-2(0.28) | HR-17 | A1 | W-1 | SG-1 | SR-1 | 100 |
| Example 7 | | | | | | | | | |
| Comparative | C-2(3.7) | Polymer(8) | PBI(0.30) | C-12 | A1/B2(90/10) | W-1 | SG-1 | SR-1 | 100 |
| Example 8 | | | | | | | | | |
| Comparative | C-3(4.0) | Polymer(6) | PEA(0.40) | C-18 | A1/B2(80/20) | W-1 | SG-1 | SR-1 | 100 |
| Example 9 | | | | | | | | | |
| Comparative | C-4(3.8) | Polymer(7) | DIA(0.31) | HR-32 | A1/B2(90/10) | W-1 | SG-1 | SR-1 | 100 |
| Example 10 | | | | | | | | | |
| Comparative | C-6(4.0) | Polymer(1) | PEA(0.40) | C-2 | A1/B2(80/20) | W-1 | SG-1 | SR-1 | 100 |
| Example 11 | | | | | | | | | |

TABLE 5

| | | | Evaluation | | | Evaluation |
|---|---|---|---|---|---|---|
| | | | Item 3 | Evaluation | Evaluation | Item 6 |
| | Evaluation | Evaluation | Development | Item 4 | Item 5 | Pattern |
| | Item 1 | Item 2 | Defects | Pattern | LWR | Collapse |
| | Sensitivity | Particles | [No./cm²] | Forming | (nm) | (nm) |
| Example 1 | 0.96 | A | 0.2 | A | 3.61 | 53.9 |
| Example 2 | 0.88 | A | 0.32 | A | 3.46 | 51.6 |
| Example 3 | 0.92 | A | 0.19 | A | 4.14 | 53.9 |
| Example 4 | 0.95 | A | 0.24 | A | 4.05 | 54 |
| Example 5 | 0.96 | A | 0.2 | A | 3.79 | 54.7 |
| Example 6 | 0.95 | A | 0.17 | A | 3.61 | 53.9 |
| Example 7 | 0.97 | A | 0.15 | A | 3.59 | 55 |
| Example 8 | 0.97 | A | 0.22 | A | 4.1 | 54 |
| Example 9 | 0.95 | A | 0.19 | A | 3.7 | 54.2 |
| Example 10 | 0.94 | A | 0.23 | A | 4.04 | 53.9 |
| Example 11 | 0.95 | A | 0.19 | A | 3.78 | 54.6 |
| Example 12 | 0.94 | A | 0.16 | A | 3.6 | 53.8 |
| Example 13 | 0.96 | A | 0.21 | A | 4.09 | 53.9 |
| Comparative | 0.73 | A | 0.22 | A | 5.72 | 35 |
| Example 1 | | | | | | |
| Comparative | 0.74 | A | 0.17 | A | 6.11 | 36.5 |
| Example 2 | | | | | | |
| Comparative | 0.72 | A | 0.2 | A | 5.66 | 39.8 |
| Example 3 | | | | | | |
| Comparative | 0.73 | A | 0.19 | A | 5.84 | 38 |
| Example 4 | | | | | | |
| Comparative | 0.71 | B | 0.2 | C | 6.43 | 30.3 |
| Example 5 | | | | | | |
| Comparative | 0.72 | A | 0.18 | A | 5.63 | 39.2 |
| Example 6 | | | | | | |
| Comparative | 0.82 | C | 1.13 | B | 4.72 | 34.7 |
| Example 7 | | | | | | |
| Comparative | 0.83 | C | 1.73 | B | 5.23 | 35.9 |
| Example 8 | | | | | | |
| Comparative | 0.85 | D | 1.32 | B | 4.64 | 37.8 |
| Example 9 | | | | | | |
| Comparative | 0.82 | D | 1.33 | B | 5.22 | 36.2 |
| Example 10 | | | | | | |
| Comparative | 0.84 | C | 1.2 | B | 4.82 | 38 |
| Example 11 | | | | | | |

From the results according to the Tables described above, it is obvious that, compared to the actinic-ray-sensitive or radiation-sensitive resin compositions of Comparative Examples 1 to 11 which use the compounds (C-1) to (C-6) which do not have the characteristics of the compound (A), the actinic-ray-sensitive or radiation-sensitive resin composition of the present invention satisfies all of excellent preservation stability of a resist solution (particularly, sensitivity and particle generation), few development defects after being stored for long periods, little line width roughness (LWR) and pattern collapse, and a favorable shape at the same time.

Furthermore, it was understood that, in a case where the actinic-ray-sensitive or radiation-sensitive resin composition of the present invention contains a compound which is represented by General Formula (1′) as the compound (A), the line width roughness (LWR) is further reduced.

What is claimed is:

1. An actinic-ray-sensitive or radiation-sensitive resin composition comprising:
a compound (A) which generates acid by being irradiated with actinic rays or radiation where,
when relative light absorbance is εᵣ using triphenyl sulfonium nonaphlate as a reference and relative quantum efficiency is φᵣ using triphenyl sulfonium nonaphlate as a reference,
the relative light absorbance εᵣ is 0.4 to 0.8 and εᵣ×φᵣ is 0.5 to 1.0.

2. The actinic-ray-sensitive or radiation-sensitive resin composition according to claim 1,
wherein the compound (A) is a compound represented by General Formula (1) below; 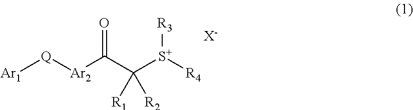
in General Formula (1), Ar₁ and Ar₂ each independently represents an aromatic ring group which has an aromatic ring with 6 to 18 carbon atoms, Ar₁ and Ar₂ may form a ring structure by bonding with each other, Q represents a hetero atom, R₁ and R₂ each independently represents a hydrogen atom, an alkyl group, a cycloalkyl group, a halogen atom, a cyano group, or an aryl group, R₃ and R₄ each independently represents an alkyl group, a cycloalkyl group, a halogen atom, a cyano group, or an aryl group, R₃ and R₄ may form a ring structure by bonding with each other and the ring structure may include an oxygen atom, a sulfur atom, a ketone group, an ester bond, or an amide bond, and X⁻ represents a non-nucleophilic anion.

3. The actinic-ray-sensitive or radiation-sensitive resin composition according to claim 2,
wherein Ar₁ and Ar₂ represent benzene ring groups in General Formula (1).

4. The actinic-ray-sensitive or radiation-sensitive resin composition according to claim 2,
wherein one of R₁ and R₂ represents a hydrogen atom and the other represents an alkyl group or a cycloalkyl group in General Formula (1).

5. The actinic-ray-sensitive or radiation-sensitive resin composition according to claim 3,
wherein one of R₁ and R₂ represents a hydrogen atom and the other represents an alkyl group or a cycloalkyl group in General Formula (1).

6. The actinic-ray-sensitive or radiation-sensitive resin composition according to claim 1,
wherein the compound (A) is a compound which is represented by General Formula (1′); 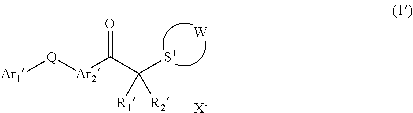
in General Formula (1′), R₁′ is the same as R₁ in General Formula (1). R₂′ is the same as R₂ in General Formula (1), Ar₁′ is the same as Ar₁ in General Formula (1), Ar₂′ is the same as Ar₂ in General Formula (1), W includes an oxygen atom, a sulfur atom, or a nitrogen atom and represents a divalent group which forms a cyclic structure by bonding with sulfonium cations, and X⁻ represents a non-nucleophilic anion.

7. The actinic-ray-sensitive or radiation-sensitive resin composition according to claim 2,
wherein X⁻ in General Formula (1) is a non-nucleophilic anion which is represented by General Formula (2) below; 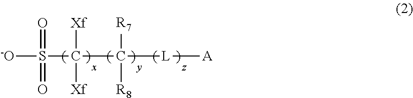
in General Formula (2), a plurality of Xf each independently represents a fluorine atom or an alkyl group which is substituted with at least one fluorine atom, R₇ and R₈ each independently represents a hydrogen atom, a fluorine atom, or an alkyl group and R₇ and R₈ may be the same or may be different in a case where a plurality of R₇ and R₈ are present, L represents a divalent bonding group and L may be the same or may be different in a case where a plurality of L are present, A represents a cyclic organic group, x represents an integer of 1 to 20, y represents an integer of 0 to 10, and z represents an integer of 0 to 10.

8. The actinic-ray-sensitive or radiation-sensitive resin composition according to claim 6,
wherein X⁻ in General Formula (1) is a non-nucleophilic anion which is represented by General Formula (2) below; 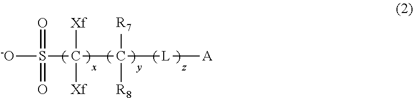
in General Formula (2), a plurality of Xf each independently represents a fluorine atom or an alkyl group which is substituted with at least one fluorine atom, R₇ and R₈ each independently represents a hydrogen atom, a fluorine atom, or an alkyl group and R₇ and R₈ may be the same or may be different in a case where a plurality of R₇ and R₈ are present, L represents a divalent bonding group and L may be the same or may be different in a case where a plurality of L are present, A represents a cyclic organic group, x represents an integer of 1 to 20, y represents an integer of 0 to 10, and z represents an integer of 0 to 10.

9. The actinic-ray-sensitive or radiation-sensitive resin composition according to claim 1, further comprising:
a resin which is decomposed by an action of an acid and which has increased solubility with respect to an alkaline developing solution.

10. The actinic-ray-sensitive or radiation-sensitive resin composition according to claim 1, further comprising:
a low molecular compound which has a nitrogen atom and a group which leaves by an action of an acid or a basic compound.

11. The actinic-ray-sensitive or radiation-sensitive resin composition according to claim 1, further comprising:
a basic compound where basicity decreases or disappears due to irradiation with actinic rays or radiation.

12. A resist film which is formed using the actinic-ray-sensitive or radiation-sensitive resin composition according to claim 1.

13. The resist film according to claim 12,
wherein the thickness of the film is 80 nm or less.

14. A pattern forming method comprising:
exposing the resist film according to claim 12; and
developing the exposed resist film.

15. The pattern forming method according to claim 14,
wherein an exposure method is a liquid immersion exposure method.

16. A producing method of an electronic device comprising:
the pattern forming method according to claim 14.

17. An electronic device manufactured by the producing method of an electronic device according to claim 16.
